SEQUENCE LISTING (88 sequences)

SEQ ID NO: 1 - skipped

SEQ ID NO: 2 - protein, 21 aa, synthetic construct
  REGION 1..21: note = synthetic
LLLVTSLLLCELPHPAFLLIP

SEQ ID NO: 3 - protein, 25 aa, synthetic construct
  REGION 1..25: note = synthetic
QVQLQQSGPGLVKPSQTLSLTCAIS

SEQ ID NO: 4 - protein, 10 aa, synthetic construct
  REGION 1..10: note = synthetic
GDSVSSNSAA

SEQ ID NO: 5 - protein, 17 aa, synthetic construct
  REGION 1..17: note = synthetic
WNWIRQSPSRGLEWLGR

SEQ ID NO: 6 - protein, 9 aa, synthetic construct
  REGION 1..9: note = synthetic
TYYRSKWYN

SEQ ID NO: 7 - protein, 38 aa, synthetic construct
  REGION 1..38: note = synthetic
DYAVSVKSRITINPDTSKNQFSLQLNSVTPEDTAVYYC

SEQ ID NO: 8 - protein, 14 aa, synthetic construct
  REGION 1..14: note = synthetic
AREVTGDLEDAFDI

SEQ ID NO: 9 - protein, 11 aa, synthetic construct
  REGION 1..11: note = synthetic
WGQGTMVTVSS

SEQ ID NO: 10 - protein, 5 aa, synthetic construct
  REGION 1..5: note = synthetic
GGGGS

SEQ ID NO: 11 - protein, 26 aa, synthetic construct [sequence omitted]

SEQ ID NO: 12 - protein, 6 aa, synthetic construct
  REGION 1..6: note = synthetic
QTIWSY

SEQ ID NO: 13 - protein, 17 aa, synthetic construct
  REGION 1..17: note = synthetic
LNWYQQRPGKAPNLLIY

SEQ ID NO: 14 - skipped

SEQ ID NO: 15 - protein, 36 aa, synthetic construct
  REGION 1..36: note = synthetic
SLQSGVPSRFSGRGSGTDFTLTISSLQAEDFATYYC

SEQ ID NO: 16 - protein, 9 aa, synthetic construct
  REGION 1..9: note = synthetic
QQSYSIPQT

SEQ ID NO: 17 - protein, 10 aa, synthetic construct
  REGION 1..10: note = synthetic
FGQGTKLEIK

SEQ ID NO: 18 - protein, 47 aa, synthetic construct
  REGION 1..47: note = synthetic
TSTTTPAPRPPTPAPTIASQPLSLRPEACRPAAGGAVHTRGLDFACD

SEQ ID NO: 19 - protein, 24 aa, synthetic construct
  REGION 1..24: note = synthetic
IYIWAPLAGTCGVLLLSLVITLYC

SEQ ID NO: 20 - protein, 42 aa, synthetic construct
  REGION 1..42: note = synthetic
KRGRKKLLYIFKQPFMRPVQTTQEEDGCSCRFPEEEEGGCEL

SEQ ID NO: 21 - protein, 112 aa, synthetic construct
  REGION 1..112: note = synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 22 aa, synthetic construct
  REGION 1..22: note = synthetic
GSGEGRGSLLTCGDVEENPGPR

SEQ ID NO: 23 - protein, 26 aa, synthetic construct
  REGION 1..26: note = synthetic
DIQMTQTTSSLSASLGDRVTISCRAS

SEQ ID NO: 24 - protein, 6 aa, synthetic construct
  REGION 1..6: note = synthetic
QDISKY

SEQ ID NO: 25 - protein, 17 aa, synthetic construct
  REGION 1..17: note = synthetic
LNWYQQKPDGTVKLLIY

SEQ ID NO: 26 - skipped

SEQ ID NO: 27 - protein, 36 aa, synthetic construct
  REGION 1..36: note = synthetic
RLHSGVPSRFSGSGSGTDYSLTISNLEQEDIATYFC

SEQ ID NO: 28 - protein, 9 aa, synthetic construct
  REGION 1..9: note = synthetic
QQGNTLPYT

SEQ ID NO: 29 - protein, 10 aa, synthetic construct
  REGION 1..10: note = synthetic
FGGGTKLEIT

SEQ ID NO: 30 - protein, 18 aa, synthetic construct
  REGION 1..18: note = synthetic
GSTSGSGKPGSGEGSTKG

SEQ ID NO: 31 - protein, 25 aa, synthetic construct
  REGION 1..25: note = synthetic
EVKLQESGPGLVAPSQSLSVTCTVS

SEQ ID NO: 32 - protein, 8 aa, synthetic construct
  REGION 1..8: note = synthetic
GVSLPDYG

SEQ ID NO: 33 - protein, 17 aa, synthetic construct
  REGION 1..17: note = synthetic
VSWIRQPPRKGLEWLGV

SEQ ID NO: 34 - protein, 7 aa, synthetic construct
  REGION 1..7: note = synthetic
IWGSETT

SEQ ID NO: 35 - protein, 38 aa, synthetic construct
  REGION 1..38: note = synthetic
YYNSALKSRLTIIKDNSKSQVFLKMNSLQTDDTAIYYC

SEQ ID NO: 36 - protein, 14 aa, synthetic construct
  REGION 1..14: note = synthetic
AKHYYYGGSYAMDY

SEQ ID NO: 37 - protein, 11 aa, synthetic construct
  REGION 1..11: note = synthetic
WGQGTSVTVSS

SEQ ID NO: 38 - protein, 12 aa, synthetic construct
  REGION 1..12: note = synthetic
RKRRGSGTPDPW

SEQ ID NO: 39 - protein, 24 aa, synthetic construct
  REGION 1..24: note = synthetic
GSGATNFSLLKQAGDVEENPGPLE

SEQ ID NO: 40 - protein, 44 aa, synthetic construct
  REGION 1..44: note = synthetic
TSAAAIEVMYPPPYLDNEKSNGTIIHVKGKHLCPSPLFPGPSKP

SEQ ID NO: 41 - protein, 27 aa, synthetic construct
  REGION 1..27: note = synthetic
FWVLVVVGGVLACYSLLVTVAFIIFWV

SEQ ID NO: 42 - protein, 41 aa, synthetic construct
  REGION 1..41: note = synthetic
RSKRSRLLHSDYMNMTPRRPGPTRKHYQPYAPPRDFAAYRS

SEQ ID NO: 43 - protein, 21 aa, synthetic construct
  REGION 1..21: note = synthetic
GSGEGRGSLLTCGDVEENPGP

SEQ ID NO: 44 - protein, 22 aa, synthetic construct
  REGION 1..22: note = synthetic
GSGATNFSLLKQAGDVEENPGP

SEQ ID NO: 45 - protein, 23 aa, synthetic construct
  REGION 1..23: note = synthetic
GSGQCTNYALLKLAGDVESNPGP

SEQ ID NO: 46 - protein, 25 aa, synthetic construct
  REGION 1..25: note = synthetic
GSGVKQTLNFDLLKLAGDVESNPGP

SEQ ID NO: 47 - protein, 4 aa, synthetic construct
  REGION 1..4: note = synthetic
RKRR

SEQ ID NO: 48 - protein, 997 aa, synthetic construct
  REGION 1..997: note = synthetic
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPRMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 1008 aa, synthetic construct
  REGION 1..1008: note = synthetic
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPLEMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 1008 aa, synthetic construct
  REGION 1..1008: note = synthetic
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPLEMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 1009 aa, synthetic construct
  REGION 1..1009: note = synthetic
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPLEMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 1007 aa, synthetic construct
  REGION 1..1007: note = synthetic
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRKRRGSGTPDPWGSGATNFSLLKQAGDVEENPGPLEMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 2994 bp, synthetic construct
  misc_feature 1..2994: note = synthetic
  source 1..2994: mol_type = other DNA
atgctgctgctcgtgacatctctgctgctgtgcgagctgccccaccccgcctttctgctgattcctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaagactagtaccaccacccctgcccctagacctcccaccccagccccaacaattgccagccagcctctgtctctgcggcccgaagcttgtagacctgctgccggcggagccgtgcacaccagaggactggatttcgcctgcgacatctacatctgggcccctctggccggcacatgtggcgtgctgctgctgagcctggtcatcaccctgtactgcaagcggggcagaaagaagctgctgtacatcttcaagcagcccttcatgcggcccgtgcagaccacacaggaagaggacggctgcagctgccggttccctgaggaagaagaaggcggctgcgaactgagagtgaagttcagcagaagcgccgacgcccctgcctaccagcagggccagaaccagctgtacaacgagctgaacctgggcagacgggaagagtacgacgtgctggacaagcggagaggcagagatcccgagatgggcggcaagcccagacggaagaatccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctacgatgccctgcacatgcaggccctgcctccaagaggcagcggagagggcagaggcagcctgctgacctgcggggacgtggaagagaacccaggccccagaatgctgctcctggtcacctctctgctgctgtgcgagctgccccaccccgcctttctgctgatccccgacatccagatgacccagaccaccagcagcctgagcgccagcctgggcgatagagtgaccatcagctgcagagccagccaggacatcagcaagtacctgaactggtatcagcagaaacccgatggcaccgtgaaactgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcaccggcagcacaagcggctctggcaagcctggatctggcgagggctctaccaagggcgaagtgaagctgcaggaaagcggccctggactggtggccccatctcagagcctgtccgtgacctgtaccgtgtccggcgtgtccctgcccgattatggcgtgtcctggatccggcagcctcccagaaagggcctggaatggctgggcgtgatctggggcagcgagacaacctactacaacagcgccctgaagtcccggctgaccatcatcaaggacaactccaagagccaggtgttcctgaagatgaactccctgcagaccgacgacaccgccatctactactgcgccaagcactactactacggcggcagctacgccatggactactggggccagggcaccagcgtgaccgtgtcatcttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccaacagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 54 - DNA, 3027 bp, synthetic construct
  misc_feature 1..3027: note = synthetic
  source 1..3027: mol_type = other DNA
atggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagtccggaaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactgagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccctcgagatggagtttggcctgagctggctgttcctggtggccatcctcaagggcgtgcagtgctccagggacatccagatgacccagaccacaagcagcctgagcgcttccctcggcgacagggtgaccatctcctgtagagcctcccaagacatctccaagtacctgaactggtaccagcagaaacccgacggcaccgtgaagctgctgatctaccacaccagcaggctgcattccggcgtgccctccagattttccggcagcggctctggtaccgactacagcctcaccatcagcaacttagaacaggaggacatcgccacatatttctgccaacagggaaacacactcccctataccttcggcggcggcacaaagttagaaatcaccggctccacatccggcagcggaaaacctggttctggcgagggcagcaccaagggcgaagtgaagctgcaggaaagcggacctggactggtcgctcccagccagagcctcagcgtgacctgtacagtgagcggcgtgagcctgcctgattacggcgtgagctggattagacagcctcccaggaagggcttagaatggctcggcgtgatttggggcagcgagacaacctactataacagcgccctgaagagcaggctcaccattatcaaggacaacagcaaatcccaggtcttcctgaagatgaacagcctccagaccgacgacaccgccatctactactgcgccaagcactactattatggcggctcctacgccatggactactggggccagggcaccagcgtgacagtgagctccactagtgccgccgctatcgaagtgatgtaccctcctccctacctggacaacgagaagtccaacggcaccatcatccacgtgaagggcaagcacctgtgccccagccctctgttccctggccctagcaagcctttctgggtgctggtggtcgtgggcggcgtgctggcctgttactctctgctggtcacagtggccttcatcatcttttgggtccgaagcaagcggagccggctgctgcacagcgactacatgaacatgacccctcggaggccaggccccaccagaaagcactaccagccctacgcccctccccgggactttgccgcctatcggagccgcgtgaagttctccagatccgctgatgctcccgcttatcagcaggggcagaatcagctctataatgaactgaatctggggcggagagaggaatacgacgtcctcgataagaggcgcggcagggaccctgagatgggaggaaaacctcggagaaaaaaccctcaggaagggctctacaacgagctgcagaaagataagatggctgaagcttactccgaaattgggatgaagggtgaaaggcggaggggcaaaggacacgacggactgtatcagggactgtccacagccacaaaagatacctatgacgcactccatatgcaggctctcccacccagatga

SEQ ID NO: 55 - DNA, 3024 bp, synthetic construct
  misc_feature 1..3024: note = synthetic
  source 1..3024: mol_type = other DNA
atggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagtccggagccgctgctattgaagtgatgtacccccccccctacctggataacgagaagtccaacggcaccatcatccacgtgaagggaaagcatctgtgtcccagccctctgttccccggacccagcaagcccttctgggtcctcgtcgtcgtgggaggcgtgctggcctgctacagcctgctggtcaccgtcgccttcatcatcttctgggtgaggtccaagaggagcagactgctgcacagcgattacatgaacatgacccccagaaggcctggacctaccagaaagcactaccagccttatgctcctcccagagactttgccgcctacagatccagagtgaagtttagcaggagcgctgatgcccccgcctatcagcagggccagaaccagctctacaacgagctgaacctgggcagaagggaggaatatgatgtgctggacaagagaagaggcagggaccccgagatgggcggcaagcctaggaggaaaaatcctcaagagggcctgtacaacgagctccaaaaggataagatggccgaggcctattccgagatcggcatgaaaggcgagaggagaaggggaaagggacacgacggcctgtatcaaggcctgtccacagccaccaaagacacctatgatgccctgcacatgcaggctctgcctcctaggaggaagaggaggggttctggcacacccgacccttggggttctggtgccaccaacttctccctgctgaaacaggccggagacgtcgaagagaatcctggacccctcgagatggagtttggactgtcctggctgtttctcgtcgccatcctgaaaggcgtgcaatgctccagagacatccaaatgacccagacaacatcctccctcagcgccagcctgggcgacagagtgaccatttcctgcagagcttcccaggacatcagcaagtacctgaactggtatcagcagaagcccgacggcaccgtgaagctcctcatctatcacaccagcaggctccattccggcgtgcctagcaggttcagcggaagcggcagcggaaccgactactccctgaccatcagcaatttagaacaagaggatatcgccacctacttttgccagcagggaaacacactgccttacacctttggcggcggcaccaaactggagattacaggttctacctccggcagcggcaaacccggaagcggcgagggcagcacaaagggagaagtcaaactgcaggagagcggccctggactggtggctcctagccagtccctgtccgtgacctgcacagtgagcggagtcagcctgcctgattacggcgtcagctggattaggcagccccccagaaagggactggagtggctcggcgtgatttggggctccgaaaccacctactacaactccgctctgaagagcaggctgaccatcattaaagataactccaagtcccaggtgttcctgaagatgaactccctgcagacagatgacaccgccatctattactgcgccaagcactactactacggaggcagctacgccatggattactggggccagggcacctccgtgaccgtgtccagcactagtaccacaacccctgctcctagaccccctacacctgcccccaccattgccagccagcccctgtccctgaggcctgaagcctgcagacccgctgctggaggagctgtgcacaccaggggactggacttcgcctgcgacatctacatctgggcccccctggccggaacctgtggcgtgctgctgctgagcctggtcatcacactgtactgcaagaggggcaggaaaaagctgctctacatcttcaagcagcctttcatgaggcctgtccagacaacccaagaggaggatggctgcagctgtaggttccctgaggaggaggaaggcggctgcgagctcagagtgaaattcagcagatccgctgatgctcccgcttaccagcagggacagaaccagctgtataacgaactgaatctgggaaggagggaggaatacgacgtgctggataaaaggaggggacgtgatcctgaaatgggaggcaagcccagaagaaaaaatccccaggagggcctgtataatgagctccagaaagacaagatggccgaagcctactccgagattggcatgaaaggagagagaaggagaggcaaaggccatgatggcctctaccagggcctgtccaccgctacaaaggacacctacgacgccctccatatgcaggccctgccccccagg

SEQ ID NO: 56 - DNA, 3027 bp, synthetic construct
  misc_feature 1..3027: note = synthetic
  source 1..3027: mol_type = other DNA
atggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagtccggaaccaccacccccgcccctaggcctcccacacctgcccccacaatcgcctcccagcctctcagcctgaggcctgaagcttgcaggcccgctgccggaggagctgtccataccaggggactcgacttcgcctgcgacatttacatttgggcccctctggctggaacctgcggagtcctgctgctgtccctggtgatcacactgtactgtaagaggggcagaaagaagctgctctacatcttcaagcagccctttatgagacccgtgcagacaacccaggaggaagacggatgcagctgcaggttccctgaggaggaggagggcggctgcgaactgagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctagaaggaagagaagaggctctggtacccccgatccttggggaagcggcgctaccaacttctccctgctcaagcaggctggcgatgtggaggagaaccccggccccctcgagatggagtttggactgtcctggctgtttctcgtcgccatcctgaaaggcgtgcaatgctccagagacatccaaatgacccagacaacatcctccctcagcgccagcctgggcgacagagtgaccatttcctgcagagcttcccaggacatcagcaagtacctgaactggtatcagcagaagcccgacggcaccgtgaagctcctcatctatcacaccagcaggctccattccggcgtgcctagcaggttcagcggaagcggcagcggaaccgactactccctgaccatcagcaatttagaacaagaggatatcgccacctacttttgccagcagggaaacacactgccttacacctttggcggcggcaccaaactggagattacaggttctacctccggcagcggcaaacccggaagcggcgagggcagcacaaagggagaagtcaaactgcaggagagcggccctggactggtggctcctagccagtccctgtccgtgacctgcacagtgagcggagtcagcctgcctgattacggcgtcagctggattaggcagccccccagaaagggactggagtggctcggcgtgatttggggctccgaaaccacctactacaactccgctctgaagagcaggctgaccatcattaaagataactccaagtcccaggtgttcctgaagatgaactccctgcagacagatgacaccgccatctattactgcgccaagcactactactacggaggcagctacgccatggattactggggccagggcacctccgtgaccgtgtccagcactagtaccacaacccctgctcctagaccccctacacctgcccccaccattgccagccagcccctgtccctgaggcctgaagcctgcagacccgctgctggaggagctgtgcacaccaggggactggacttcgcctgcgacatctacatctgggcccccctggccggaacctgtggcgtgctgctgctgagcctggtcatcacactgtactgcaagaggggcaggaaaaagctgctctacatcttcaagcagcctttcatgaggcctgtccagacaacccaagaggaggatggctgcagctgtaggttccctgaggaggaggaaggcggctgcgagctcagagtgaaattcagcagatccgctgatgctcccgcttaccagcagggacagaaccagctgtataacgaactgaatctgggaaggagggaggaatacgacgtgctggataaaaggaggggacgtgatcctgaaatgggaggcaagcccagaagaaaaaatccccaggagggcctgtataatgagctccagaaagacaagatggccgaagcctactccgagattggcatgaaaggagagagaaggagaggcaaaggccatgatggcctctaccagggcctgtccaccgctacaaaggacacctacgacgccctccatatgcaggccctgccccccagg

SEQ ID NO: 57 - DNA, 3021 bp, synthetic construct
  misc_feature 1..3021: note = synthetic
  source 1..3021: mol_type = other DNA
atggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagtccggagccgctgctattgaagtgatgtacccccccccctacctggataacgagaagtccaacggcaccatcatccacgtgaagggaaagcatctgtgtcccagccctctgttccccggacccagcaagcccttctgggtcctcgtcgtcgtgggaggcgtgctggcctgctacagcctgctggtcaccgtcgccttcatcatcttctgggtgaggtccaagaggagcagactgctgcacagcgattacatgaacatgacccccagaaggcctggacctaccagaaagcactaccagccttatgctcctcccagagactttgccgcctacagatccagagtgaagtttagcaggagcgctgatgcccccgcctatcagcagggccagaaccagctctacaacgagctgaacctgggcagaagggaggaatatgatgtgctggacaagagaagaggcagggaccccgagatgggcggcaagcctaggaggaaaaatcctcaagagggcctgtacaacgagctccaaaaggataagatggccgaggcctattccgagatcggcatgaaaggcgagaggagaaggggaaagggacacgacggcctgtatcaaggcctgtccacagccaccaaagacacctatgatgccctgcacatgcaggctctgcctcctaggaggaagaggaggggttctggcacacccgacccttggggttctggtgccaccaacttctccctgctgaaacaggccggagacgtcgaagagaatcctggacccctcgagatggagtttggcctgagctggctgttcctggtggccatcctcaagggcgtgcagtgctccagggacatccagatgacccagaccacaagcagcctgagcgcttccctcggcgacagggtgaccatctcctgtagagcctcccaagacatctccaagtacctgaactggtaccagcagaaacccgacggcaccgtgaagctgctgatctaccacaccagcaggctgcattccggcgtgccctccagattttccggcagcggctctggtaccgactacagcctcaccatcagcaacttagaacaggaggacatcgccacatatttctgccaacagggaaacacactcccctataccttcggcggcggcacaaagttagaaatcaccggctccacatccggcagcggaaaacctggttctggcgagggcagcaccaagggcgaagtgaagctgcaggaaagcggacctggactggtcgctcccagccagagcctcagcgtgacctgtacagtgagcggcgtgagcctgcctgattacggcgtgagctggattagacagcctcccaggaagggcttagaatggctcggcgtgatttggggcagcgagacaacctactataacagcgccctgaagagcaggctcaccattatcaaggacaacagcaaatcccaggtcttcctgaagatgaacagcctccagaccgacgacaccgccatctactactgcgccaagcactactattatggcggctcctacgccatggactactggggccagggcaccagcgtgacagtgagctccactagtgccgccgctatcgaagtgatgtaccctcctccctacctggacaacgagaagtccaacggcaccatcatccacgtgaagggcaagcacctgtgccccagccctctgttccctggccctagcaagcctttctgggtgctggtggtcgtgggcggcgtgctggcctgttactctctgctggtcacagtggccttcatcatcttttgggtccgaagcaagcggagccggctgctgcacagcgactacatgaacatgacccctcggaggccaggccccaccagaaagcactaccagccctacgcccctccccgggactttgccgcctatcggagccgcgtgaagttctccagatccgctgatgctcccgcttatcagcaggggcagaatcagctctataatgaactgaatctggggcggagagaggaatacgacgtcctcgataagaggcgcggcagggaccctgagatgggaggaaaacctcggagaaaaaaccctcaggaagggctctacaacgagctgcagaaagataagatggctgaagcttactccgaaattgggatgaagggtgaaaggcggaggggcaaaggacacgacggactgtatcagggactgtccacagccacaaaagatacctatgacgcactccatatgcaggctctcccacccaga

SEQ ID NO: 58 - DNA, 7691 bp, synthetic construct
  misc_feature 1..7691: note = synthetic
  source 1..7691: mol_type = other DNA
gacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattgggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

SEQ ID NO: 59 - protein, 492 aa, synthetic construct
  REGION 1..492: note = synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 485 aa, synthetic construct
  REGION 1..485: note = synthetic
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKTSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 733 aa, synthetic construct
  REGION 1..733: note = synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 20 aa, synthetic construct
  REGION 1..20: note = synthetic
ALPVTALLLPLALLLHAARP

SEQ ID NO: 63 - protein, 751 aa, synthetic construct
  REGION 1..751: note = Synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSGGGGSGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 766 aa, synthetic construct
  REGION 1..766: note = Synthetic
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - protein, 749 aa, synthetic construct
  REGION 1..749: note = Synthetic
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - protein, 742 aa, synthetic construct
  REGION 1..742: note = Synthetic
ATMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - protein, 755 aa, synthetic construct
  REGION 1..755: note = Synthetic
ATMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGCGSGGGGSGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSGGGGSGGCGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 744 aa, synthetic construct
  REGION 1..744: note = Synthetic
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSTSGSGKPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 744 aa, synthetic construct
  REGION 1..744: note = Synthetic
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKTSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 747 aa, synthetic construct
  REGION 1..747: note = Synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSCGGGSQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKGGGGCGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTSSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - DNA, 2256 bp, synthetic construct
  misc_feature 1..2256: note = Synthetic
  source 1..2256: mol_type = other DNA
atgctgctgctcgtgacatctctgctgctgtgcgagctgccccaccccgcctttctgctgattcctgatattcagatgacccagaccacctccagcctgtccgccagcctgggcgatcgcgtgaccatctcttgcagagccagccaggacatcagcaagtatctgaattggtatcagcagaaacccgacggcaccgtgaagctgctgatctaccacaccagcagactgcactccggcgtgccatccagattcagcggctctggctccggcaccgattatagcctgaccatcagcaacctggaacaggaagatatcgctacctacttttgtcagcaaggcaacaccctgccctacaccttcggcggaggcacaaaactggaaattaccggcagcaccagcggcagcggaaagcctggaagcggcgagggaagcaccaagggcgaagtgaaactgcaggaaagcggacccggactggtggccccaagccagtctctgagcgtgacatgtaccgtgtccggcgtgtccctgcccgactatggcgtgtcctggatcaggcagccccccagaaagggactggaatggctgggagtgatctggggcagcgagacaacctactacaacagcgccctgaagtccaggctgaccattatcaaggacaactccaagagccaggtgttcctgaagatgaacagcctgcagaccgacgacacagccatctactattgcgccaagcactactactacggcggcagctacgccatggactactggggacagggaacctccgtgaccgtgtcctctggcggagggggatctggcggcggaggatctgggggaggcggcagtgggggcggaggaagtggcgggggaggctctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 72 - DNA, 2301 bp, synthetic construct
  misc_feature 1..2301: note = Synthetic
  source 1..2301: mol_type = other DNA
atgctgctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatccggcagagcccttctagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggcagcacaagcggctctggcaagcctggatctggcgagggctctaccaagggcgacatccagatgacccagagccccagcagcctgtctgccagcgtgggcgacagagtgaccatcacctgtagggccagccagaccatctggtcctacctgaactggtatcagcagaggcccggcaaggcccccaacctgctgatctatgccgcctccagtctgcagagcggcgtgcccagcagattcagcggcagaggcagcggcaccgacttcaccctgaccattagtagtctgcaggccgaggacttcgccacctactactgccagcagagctacagcatcccccagaccttcggccaggggaccaagctggaaatcaagggcggaggcggaagtggcggcggaggatctgggggaggcggatcaggcggagggggctcaggggggggaggctctgatattcagatgacacagaccacctccagcctgagcgcctctctgggagatcgcgtgacaatctcctgccgcgccagccaggacatcagcaagtatctgaattggtatcagcagaaacccgacggcaccgtgaagctgctgatctaccacaccagcagactgcactccggcgtgccatccagattttccggcagcggctccggcaccgattatagcctgaccatcagcaacctggaacaggaagatatcgctacctacttttgtcagcaaggcaacaccctgccctacaccttcggcggaggcacaaaactggaaattaccggctccaccagcggcagcggaaagcctggaagcggagagggaagcacaaagggcgaagtgaaactgcaggaaagcggacccggactggtggccccaagccagtctctgagcgtgacatgtaccgtgtccggcgtgtccctgcccgactatggcgtgtcctggatcagacagccccccagaaagggactggaatggctgggagtgatctggggcagcgagacaacctactacaacagcgccctgaagtccaggctgacaatcatcaaggacaactccaagagccaggtgttcctgaagatgaattccctgcagaccgacgacacagccatctactattgcgccaagcactactactacggcggcagctacgccatggactactggggacagggaacctccgtgaccgtgtcctcttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccaacagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 73 - DNA, 2250 bp, synthetic construct
  misc_feature 1..2250: note = Synthetic
  source 1..2250: mol_type = other DNA
atggctctgcctgtgacagctctgctgctgcctctggccctgctgctccatgctgctagacctcaggtgcagctccagcagtctggcccaggactggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggcggaagcgacatccagatgacccagagccctagctccctgagcgccagcgtgggcgacagagtgaccatcacctgtcgggccagccagaccatctggtcctacctgaattggtatcagcagcggccaggcaaggcccctaacctgctgatctatgccgccagcagcctgcagagcggcgtgccaagcagattctctggcagaggctccggcaccgacttcaccctgacaatcagttccctgcaggccgaggacttcgccacctactactgccagcagtcctacagcatccctcagaccttcggccaggggaccaagctggaaatcaagggtggcggaggatctggcggcggtggtagtggcggcggaggttcaggtggtggcggctccgatattcagatgacacagaccacctccagcctgtccgcctccctgggagatagagtgacaatctcctgcagggcctcccaggacatcagcaagtatctcaactggtaccagcagaaacccgacggcaccgtgaagctgctcatctaccacaccagcagactgcactccggcgtgccctctagattttccggctctggcagcggcacagactactccctgaccatctccaacctggaacaggaagatatcgctacttacttctgtcagcaaggcaacaccctgccctacaccttcggcggaggcacaaaactggaaattaccggcagcaccagcggcagcggaaagcctggaagcggcgagggctctaccaagggcgaagtgaaactgcaggaaagcggccctggcctggtggccccttctcagtctctgtccgtgacctgtaccgtgtctggcgtgtccctgcccgattacggcgtgtcctggatcaggcagcctccccggaaaggactcgaatggctcggcgtgatctggggcagcgagacaacctactacaacagcgccctgaagtccaggctgaccatcatcaaggacaactccaagagccaggtgttcctgaagatgaactctctgcagaccgacgataccgccatctattattgcgccaagcactactactacggcggcagctacgccatggactactggggacagggaacctccgtgaccgtgtccagtactagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccaacagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 74 - DNA, 2247 bp, synthetic construct
  misc_feature 1..2247: note = Synthetic
  source 1..2247: mol_type = other DNA
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatccggcggcggaggatccggcggcggaggatcccaggtgcagctgcagcagtctggacccggcctcgtgaagcctagccagaccctgtctctgacctgcgccatcagcggcgatagcgtgtccagcaatagcgccgcctggaactggatccggcagagcccttctagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcgggggaggcggcagcgatattcagatgacacagagcccctccagcctgtccgcctctgtgggagacagagtgacaatcacctgtcgggcctcccagaccatctggtcctatctgaattggtatcagcagcggcctggcaaggcccccaacctgctgatctatgccgccagctctctgcagtccggcgtgccatctagattcagcggcagaggcagcggcaccgatttcaccctgacaattagcagtctgcaggccgaggacttcgccacctactattgccagcagagctacagcatcccccagaccttcggccagggaacaaaactggaaatcaaagggggaggcggcagcgggggaggcggcagcgggggaggcggcagcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctactagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatcctaatgatcagtcgac

SEQ ID NO: 75 - DNA, 2274 bp, synthetic construct
  misc_feature 1..2274: note = Synthetic
  source 1..2274: mol_type = other DNA
atgctgctgctcgtgacaagcctgctgctgtgcgagctgccccaccctgcctttctgctgatccccgacatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggctgcggatccggcggcggaggatccggcggcggaggatcccaggtgcagctgcagcagtctggacccggcctcgtgaagcctagccagaccctgtctctgacctgcgccatcagcggcgatagcgtgtccagcaatagcgccgcctggaactggatccggcagagcccttctagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggcagcacaagcggctctggcaagcctggatctggcgagggctctaccaagggcgatattcagatgacacagagcccctccagcctgtccgcctctgtgggagacagagtgacaatcacctgtcgggcctcccagaccatctggtcctatctgaattggtatcagcagcggcctggcaaggcccccaacctgctgatctatgccgccagctctctgcagtccggcgtgccatctagattcagcggcagaggcagcggcaccgatttcaccctgacaattagcagtctgcaggccgaggacttcgccacctactattgccagcagagctacagcatcccccagaccttcggccagggaacaaaactggaaatcaaagggggaggcggcagcgggggaggcggcagcgggggatgcggcagcgaagtgaaactgcaggaatctggccctggcctggtggccccaagccagtctctgagcgtgacctgtaccgtgtctggcgtgtccctgcccgattacggcgtgtcctggatcagacagccccccagaaagggactggaatggctgggagtgatctggggcagcgagacaacctactacaacagcgccctgaagtccaggctgaccatcatcaaggacaactccaagagccaggtgttcctgaagatgaattccctgcagaccgacgacaccgccatctattactgtgccaagcactactactacggcggcagctacgccatggactactggggacagggaacctccgtgaccgtgtcctctactagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatcctaa

SEQ ID NO: 76 - DNA, 2235 bp, synthetic construct
  misc_feature 1..2235: note = Synthetic
  source 1..2235: mol_type = other DNA
atggctctgcctgtgacagctctgctgctgcctctggccctgctgctccatgccgccagacccgatatccagatgacccagaccaccagcagcctgagcgccagcctgggcgatagagtgaccatcagctgccgggccagccaggacatcagcaagtacctgaactggtatcagcagaaacccgacggcaccgtgaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggatctcaggtccagctccagcagtctggacccggcctggtcaagcctagccagaccctgagcctgacctgcgccatcagcggcgacagcgtgtcctctaacagcgccgcctggaattggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggcagcacaagcggctctggcaagcctggaagcggcgagggctctaccaagggcgacattcagatgactcagagcccctccagcctgtccgcctctgtgggagacagagtgacaatcacctgtagagcctcccagaccatctggtcctatctcaattggtaccagcagcggccaggcaaggcccctaacctgctcatctatgccgcctctagcctgcagtccggcgtgccatctagattcagcggcaggggcagcggcaccgatttcaccctgacaatcagttccctgcaggccgaggacttcgccacctactattgccagcagtcctacagcatccctcagaccttcggccagggaacaaagctcgaaatcaaaggtggtggtggcagtggtggcggcggaagcgaagtgaaactgcaggaaagcggccctggcctggtggccccttctcagtctctgtccgtgacctgtaccgtgtctggcgtgtccctgcccgattacggcgtgtcctggatcaggcagccaccccggaaaggactcgaatggctcggcgtgatctggggcagcgagacaacctactacaacagcgccctgaagtccaggctgaccatcatcaaggacaactccaagagccaggtgttcctgaagatgaacagcctgcagaccgacgataccgccatctattactgtgccaagcactactactacggcggcagctacgccatggactactggggacagggaacctccgtgaccgtgtccagtactagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 77 - DNA, 2238 bp, synthetic construct
  misc_feature 1..2238: note = Synthetic
  source 1..2238: mol_type = other DNA
atgctgctgctggtcacaagcctgctgctgtgcgagctgccccaccccgcctttctgctgattcctcaggtccagctgcagcagtctggccctggcctggtcaagcctagccagaccctgagcctgacctgcgccatcagcggcgacagcgtgtcctctaacagcgccgcctggaactggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtgtctagcggaggcggaggaagtggcggcggaggcagcgatatccagatgacccagaccaccagcagcctgagcgccagcctgggcgatagagtgaccatctcctgccgggccagccaggacatcagcaagtacctgaattggtatcagcagaagcccgacggcaccgtcaagctgctgatctaccacaccagcagactgcacagcggcgtgcccagcagattttctggcagcggctccggcaccgactacagcctgaccatcagcaacctggaacaggaagatatcgctacctacttctgtcagcaaggcaacaccctgccctacaccttcggaggcggcaccaagctggaaatcaccggcagcacaagcggcagcggcaagcctggatctggcgagggctctaccaagggcgaagtgaagctgcaggaaagcggacccggactggtggcccctagccagtctctgtctgtgacctgtaccgtgtccggcgtgtccctgcccgattatggcgtgtcctggatcaggcagcctccccggaaaggactcgaatggctcggcgtgatctggggcagcgagacaacctactacaacagcgccctgaagtccaggctgacaatcatcaaggacaactccaagagccaggtgttcctgaagatgaacagcctgcagaccgacgataccgccatctactattgtgccaagcactactactacggcggcagctacgccatggactactggggacagggaacctccgtgaccgtcagtagtggtggtggcggatctggtggaggcggctccgacattcagatgactcagagcccttccagcctgtccgcctctgtgggagacagagtgacaatcacctgtagagcctcccagaccatctggtcctatctcaactggtaccagcagcggccaggcaaggcccctaacctgctcatctatgccgcctctagcctgcagtccggggtgccctctagattcagcggcagaggaagcggcaccgatttcaccctgacaattagctcactgcaggccgaggacttcgccacctattactgccagcagagctacagcatccctcagaccttcggccagggaacaaagctcgaaatcaagactagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 78 - DNA, 2244 bp, synthetic construct
  misc_feature 1..2244: note = Synthetic
  source 1..2244: mol_type = other DNA
atgctgctgctggtcacaagcctgctgctgtgcgagctgccccaccccgcctttctgctgatccccgacatccagatgacccagaccaccagcagcctgagcgccagcctgggcgatagagtgaccatcagctgccgggccagccaggacatcagcaagtacctgaactggtatcagcagaagcccgacggcaccgtcaagctgctgatctaccacaccagcagactgcacagcggcgtgcccagcagattttctggcagcggctccggcaccgactacagcctgaccatctccaacctggaacaggaagatatcgctacctacttctgtcagcaaggcaacaccctgccctacaccttcggcggaggcaccaagctggaaatcacaggcggcggaggatctggcggaggcggaagttgtggtggcggatctcaggtccagctgcagcagagtggccctggcctggtcaagcctagccagaccctgagcctgacctgcgccatcagcggcgacagcgtgtcctctaacagcgccgcctggaattggatcagacagagccccagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccctgaggacaccgccgtgtactactgcgccagagaagtgaccggcgacctggaagatgccttcgacatctggggccagggcaccatggtcaccgtcagtagtggaggcggtggcagcggtggcggcggaagcgatattcagatgactcagagcccctccagcctgtccgcctctgtgggagacagagtgacaatcacctgtagagcctcccagaccatctggtcctatctcaattggtaccagcagcggccaggcaaggcccctaacctgctcatctatgccgcctctagcctgcagtccggcgtgccatctagattcagcggcagaggcagcggcaccgatttcaccctgacaatcagttccctgcaggccgaggacttcgccacctactattgccagcagagctacagcatccctcagaccttcggccagggaacaaagctcgaaatcaaaggtggaggcggctgcggaggtggtggatctggaggcggaggctccgaagtgaagctgcaggaaagcggcccaggactggtggcccctagccagtctctgtccgtgacctgtaccgtgtctggcgtgtccctgcccgattacggcgtgtcctggatcaggcagccaccccggaaaggactcgaatggctcggcgtgatctggggcagcgagacaacctactacaacagcgccctgaagtccagactgaccatcatcaaggacaactccaagagccaggtgttcctgaagatgaacagcctgcagaccgacgataccgccatctattactgtgccaagcactactactacggcggcagctacgccatggactactggggacagggaacctccgtgaccgtgtccagtactagttccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 79 - protein, 25 aa, synthetic construct
  REGION 1..25: note = Synthetic
GGGGSGGGGSGGGGSGGGGSGGGGS

SEQ ID NO: 80 - protein, 15 aa, synthetic construct
  REGION 1..15: note = Synthetic
GGGGSGGGGSGGGGS

SEQ ID NO: 81 - protein, 15 aa, synthetic construct
  REGION 1..15: note = Synthetic
GGCGSGGGGSGGGGS

SEQ ID NO: 82 - protein, 15 aa, synthetic construct
  REGION 1..15: note = Synthetic
GGGGSGGGGSGGCGS

SEQ ID NO: 83 - protein, 10 aa, synthetic construct
  REGION 1..10: note = Synthetic
GGGGSGGGGS

SEQ ID NO: 84 - protein, 15 aa, synthetic construct
  REGION 1..15: note = Synthetic
GGGGSGGGGSCGGGS

SEQ ID NO: 85 - protein, 15 aa, synthetic construct
  REGION 1..15: note = Synthetic
GGGGCGGGGSGGGGS

SEQ ID NO: 86 - protein, 47 aa, synthetic construct
  REGION 1..47: note = synthetic
SGTTTPAPRPPTPAPTIASQPLSLRPEACRPAAGGAVHTRGLDFACD

SEQ ID NO: 87 - protein, 20 aa, synthetic construct
  REGION 1..20: note = synthetic
EFGLSWLFLVAILKGVQCSR

SEQ ID NO: 88 - protein, 20 aa, synthetic construct
  REGION 1..20: note = synthetic
GGGGSGGGGSGGGGSGGGGS

(19) United States
(12) Patent Application Publication
(10) Pub. No.: US 2025/0034244 A1
(43) Pub. Date: Jan. 30, 2025

(54) BICISTRONIC CHIMERIC ANTIGEN RECEPTORS AND THEIR USES

(71) Applicant: The United States of America,as represented by the Secretary,Department of Health and Human Services, Bethesda, MD (US)

(72) Inventors: Haiying Qin, Potomac, MD (US); Crystal L. Mackall, Stanford, CA (US); Terry J. Fry, Bethesda, MD (US)

(73) Assignee: The United States of America,as represented by the Secretary,Department of Health and Human Services, Bethesda, MD (US)

(21) Appl. No.: 18/644,724

(22) Filed: Apr. 24, 2024

Related U.S. Application Data
(63) Continuation of application No. 16/613,187, filed on Nov. 13, 2019, now Pat. No. 11,980,640.
(60) Provisional application No. 62/506,268, filed on May 15, 2017.

(51) Int. Cl. C07K 16/28; A61K 39/00; A61P 35/00; C07K 14/395; C07K 14/725; C12N 15/85

(52) U.S. Cl. CPC C07K 16/2803; A61K 39/4611; A61K 39/4631; A61K 39/464412; A61K 39/464413; A61K 2239/31; A61K 2239/38; A61K 2239/48; A61P 35/00; C07K 14/395; C07K 14/7051; C12N 15/85

(57) ABSTRACT

An embodiment of the invention provides chimeric antigen receptor (CAR) amino acid constructs. Nucleic acids, recombinant expression vectors, host cells, populations of cells, and pharmaceutical compositions relating to the CAR constructs are disclosed. Methods of detecting the presence of cancer in a mammal and methods of treating or preventing cancer in a mammal are also disclosed. Methods of making the CAR constructs are disclosed.

### CROSS-REFERENCE TO RELATED APPLICATIONS

This patent application is a continuation of U.S. application Ser. No. 16/613,187, filed Nov. 13, 2019, which is the U.S. National Phase of International Patent Application No. PCT/US2018/032809, filed May 15, 2018, which claims the benefit of U.S. Provisional Patent Application No. 62/506,268, filed May 15, 2017, each of which is incorporated by reference herein in its entirety.

### STATEMENT REGARDING FEDERALLY SPONSORED RESEARCH AND DEVELOPMENT

This invention was made with Government support under project number Z01 BC011565 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: one 160,724 Byte XML file named “770572.XML” dated Apr. 15, 2024.

### BACKGROUND OF THE INVENTION

Cancer is a public health concern. Despite advances in treatments such as chemotherapy, the prognosis for many cancers, including hematological malignancies, may be poor. Accordingly, there exists an unmet need for additional treatments for cancer, particularly hematological malignancies.

### BRIEF SUMMARY OF THE INVENTION

An embodiment of the invention provides a chimeric antigen receptor (CAR) amino acid construct comprising (a) a cleavable domain; (b) a first CAR comprising a first antigen binding domain, a first transmembrane domain, and a first intracellular T cell signaling domain; and (c) a second CAR comprising a second antigen binding domain, a second transmembrane domain, and a second intracellular T cell signaling domain; wherein the first and second CARs are linked through the cleavable domain, wherein the first antigen binding domain comprises an antigen binding domain of the m971 antibody, wherein when the first CAR is cleaved from the construct, the first antigen binding domain has antigenic specificity for CD22.

Another embodiment of the invention provides a chimeric antigen receptor (CAR) amino acid construct comprising (a) a cleavable domain; (b) a first CAR comprising a first antigen binding domain, a first transmembrane domain, and a first intracellular T cell signaling domain; and (c) a second CAR comprising a second antigen binding domain, a second transmembrane domain, and a second intracellular T cell signaling domain; wherein the first and second CARs are linked through the cleavable domain, wherein the first antigen binding domain comprises an antigen binding domain of the FMC63 antibody, wherein when the first CAR is cleaved from the construct, the first antigen binding domain has antigenic specificity for CD19.

Another embodiment of the invention provides chimeric antigen receptor (CAR) amino acid construct comprising (a) two or more cleavable domains; (b) a first CAR comprising a first antigen binding domain, a first transmembrane domain, and a first intracellular T cell signaling domain; and (c) a second CAR comprising a second antigen binding domain, a second transmembrane domain, and a second intracellular T cell signaling domain; wherein the first and second CARs are linked through the two or more cleavable domains.

Another embodiment of the invention provides a method of making a chimeric antigen receptor (CAR) amino acid construct, the method comprising designing two or more cleavable domains between (a) a first CAR comprising a first antigen binding domain, a first transmembrane domain, and a first intracellular T cell signaling domain; and (b) a second CAR comprising a second antigen binding domain, a second transmembrane domain, and a second intracellular T cell signaling domain; wherein the first and second CARs are linked through the two or more cleavable domains; and cloning into a plasmid a sequence comprising from N-terminus to C-terminus the first CAR, the two or more cleavable domains, and the second CAR.

Another embodiment of the invention provides CAR amino acid constructs comprising the amino acid sequences as described herein.

Further embodiments of the invention provide related nucleic acids, recombinant expression vectors, host cells, populations of cells, and pharmaceutical compositions relating to the CAR amino acid constructs of the invention.

Additional embodiments of the invention provide methods of detecting the presence of cancer in a mammal and methods of treating or preventing cancer in a mammal.

### BRIEF DESCRIPTION OF THE DRAWINGS

FIG. 1 presents schematic diagrams of exemplary CAR constructs, in accordance with embodiments of the invention.

FIGS. 2A-2C present fluorescence-activated cell sorting dot plots comparing surface expression on human T cells of (2A) anti-CD19 CAR (the anti-CD19 CAR denoted as “CD19 CAR”) and CD3 when the T cells are transduced with a vector encoding the single anti-CD19 CAR or the V1 CAR construct (also denoted as bicistronic-V1 or bicis-V1), (2B) anti-CD22 CAR (the anti-CD22 CAR denoted as “CD22 CAR”) and CD3 when the T cells are transduced with a vector encoding the single anti-CD22 CAR or the V1 CAR construct, and (2C) anti-CD19 CAR and anti-CD22 CAR when the cells are transduced with a vector encoding the V1 CAR construct, in accordance with embodiments of the invention.

FIG. 3 presents fluorescence-activated cell sorting dot plots comparing surface expression of anti-CD19 CAR and anti-CD22 CAR on human T cells when transduced with a vector encoding the single anti-CD19 CAR, the single anti-CD22 CAR, the LoopCAR6, or co-transduced with separate vectors encoding the single anti-CD19 CAR and the single anti-CD22 CAR.

FIG. 4 presents fluorescence-activated cell sorting dot plots comparing surface expression of anti-CD19 CAR and anti-CD22 CAR on human T cells when transduced with a vector encoding the V1 CAR construct, the V5 CAR construct (also denoted as bicistronic-V5 or bicis-V5), or the LoopCAR6, in accordance with embodiments of the invention.

FIGS. 5A, 5B, 6A, 6B, 7A, 7B, and 8 are bar graphs showing in vitro activity based on cytokine production, in accordance with embodiments of the invention. FIG. 5A shows IL2 levels, and FIG. 5B shows IFNγ levels, measured when K562 cells expressing CD19, CD22, both, or none are contacted with T cells transduced with a vector encoding the V1 CAR construct, the single anti-CD19 CAR (CAR19), or the single anti-CD22 CAR (CAR22), in accordance with embodiments of the invention. FIG. 6A shows IL2 levels, and FIG. 6B shows IFNγ levels, measured when K562 cells expressing CD19, CD22, both, or none are contacted with T cells transduced with a vector encoding the V1 CAR construct, the V5 CAR construct, the LoopCAR6, the single anti-CD19 CAR, or the single anti-CD22 CAR, in accordance with embodiments of the invention. FIG. 7A shows IL2 levels, and FIG. 7B shows IFNγ levels, measured when CD19 KO and/or CD22 KO NALM6 cells are contacted with T cells transduced with a vector encoding the V1 CAR construct, the V5 CAR construct, the LoopCAR6, the single anti-CD19 CAR, or the single anti-CD22 CAR, in accordance with embodiments of the invention. FIG. 8 shows CAR T cells were co-incubated with NALM6 tumor cells for 18 hours, and the levels of IL2 production in the culture supernatant were measured by ELISA (“-N”: NALM6; “-N-19”: NALM6-CD19neg; “-N-19-22”: NALM6-CD19neg-CD22neg).

FIGS. 9-14 present bioluminescent imaging of leukemia progression in vivo after treatment using T cells transduced with a vector encoding the single anti-CD19 CAR, the single anti-CD22 CAR, the LoopCAR6, the V1 CAR construct, or the V5 CAR construct, compared to mock T cells (untransduced T cells), in accordance with embodiments of the invention. Bioluminescent intensity, as shown by increased levels of shading, represents tumor burden. “Lenti” indicates that the CAR has been designed and made within a lentiviral backbone.

FIG. 15 is a line graph showing CD22 expression in patients prior to and after loss of CD19.

FIG. 16 is a dot plot showing CD19 and CD22 expression of CRISPR CD19neg and CD22neg leukemia lines vs parental NALM6 line.

FIG. 17 presents images showing comparison of in vivo progression of CRISPR CD19neg and CD22neg leukemia cell vs parental NALM6 cell. Bioluminescent intensity, as shown by increased levels of shading, represents tumor burden.

FIG. 18 presents images showing comparison of treatment methods using CARs as described herein. NSG mice were challenged with a mixture of 2.5E5 of NALM6 and NALM6-CD19neg and NALM6-CD22neg leukemia lines on day 0. Mice in the sequential treatment group received 3E6 CAR+ on day 3 and 3E6 CAR+ T cells on day 9. Mice in the co-injection group received a total of 6E6 CAR+ T cells with 3E6 of anti-CD19 CAR+ and 3E6 of anti-CD22 CAR+ T cells on day 3. Mice in the co-transduced group received 8E6 of total T cells which contain 3E6 of anti-CD19+ and 3E6 of anti-CD22+ CAR T cells. Mice in the CD19 or CD22 groups received 3E6 of CAR+ T cells. Bioluminescent intensity, as shown by increased levels of shading, represents tumor burden. Co-injection or co-transduction of anti-CD19 and anti-CD22 CAR suggest that simultaneously targeting on both CD19 and CD22 may reduce relapse of leukemia.

FIG. 19 is a dot plot showing comparison of single vector transduction vs co-transduction with anti-CD19 and anti-CD22 CAR constructs.

FIG. 20 is a graphic plot presentation of the leukemia phenotype post CAR treatment as described in Example 7.

FIG. 21 diagrammatically presents TanCARs of Example 7, in accordance with embodiments of the invention.

FIGS. 22A and 22B are bar graphs showing cytokine production of various CARs described herein with K562, K562-CD19, K562-CD22, and K562-CD19CD22 target cell lines, in accordance with embodiments of the invention.

FIG. 23A presents images showing comparison of TanCAR1 and TanCAR4 on treatment of leukemia in vivo. NSG mice were challenged with 1E6 of luciferase-expressing NALM6 leukemia on day 0. On day 3, mice were IV injected with 3E6 of CAR expressing T cells. Bioluminescent intensity, as shown by increased levels of shading, represents tumor burden.

FIGS. 23B-23D present dot plots showing Incucyte killing assay with co-incubation of each CAR or mock T cell product, in accordance with embodiments of the invention.

FIG. 24 diagrammatically presents LoopCARs of Example 7, in accordance with embodiments of the invention.

FIGS. 25A-25C present bar graphs showing cytokine production of various CARs with K562, K562-CD19, K562-CD22, and K562-CD19CD22 target cell lines, in accordance with embodiments of the invention.

FIG. 26 is a bar graph showing cytokine production of various CARs with K562, K562-CD19, K562-CD22, and K562-CD19CD22 target cell lines, in accordance with embodiments of the invention.

FIG. 27 is a dot plot showing Incucyte killing assay with co-incubation of each CAR or mock T cell product with 10:1 of NALM6: NALM6-CD19neg cells, in accordance with embodiments of the invention.

FIG. 28 is a dot plot showing Incucyte killing assay with co-incubation of each CAR or mock T cell product with 10:1 of NALM6: NALM6-CD22neg cells, in accordance with embodiments of the invention.

FIGS. 29A-29F are bar graphs showing LoopCAR6 produces a variety of cytokines when co-Incubated with target antigens. FIG. 29A: Interferon gamma; FIG. 29B: IL6; FIG. 29C: TNF alpha; FIG. 29D: IL8; FIG. 29E: IL13; FIG. 29F: IL2.

FIG. 30 presents images. NSG mice were challenged with 1E6 of luciferase-expressing NALM6 leukemia on day 0. On day 3, mice were IV injected with 3E6 of CAR expressing T cells. Bioluminescent intensity, as shown by increased levels of shading, represents tumor burden.

FIG. 31 presents images. NSG mice were challenged with 1E6 of luciferase-expressing NALM6 leukemia on day 0. On day 3, mice were IV injected with 9E6, 3E6 and 1E6 of Loop F CAR (which is also listed herein as LoopCAR6) expressing T cells. Bioluminescent intensity, as shown by increased levels of shading, represents tumor burden.

FIG. 32 presents images. NSG mice were challenged with 1E6 of NALM6 on day 0. The mice in sequential treatment group received 3E6 CAR+ on day 3 and 3E6 CAR T cells on day 7. Mice in the co-injection group received a total of 6E6 CAR⁺ T cells with 3E6 of anti-CD19 CAR⁺ and 3E6 of anti-CD22 CAR+ T cells on day 3. Mice in the co-transduced group received 10E6 of total T cells which contain 3E6 of anti-CD19⁺ and 3E6 of anti-CD22⁺ CAR T cells. Mice in anti-CD19 or anti-CD22 groups received 3E6 of CAR T cells. Co-injection or co-transduction of anti-CD19 and anti-CD22 CAR suggest that simultaneously targeting on both CD19 and CD22 may reduce relapse of leukemia. Bioluminescent intensity, as shown by increased levels of shading, represents tumor burden.

FIG. 33A presents images. NSG mice were challenged with a mixture of 5E5 NALM6-CD19neg and 5E5 NALM6-CD22neg luciferase-expressing leukemia on day 0. On day 3, mice were treated with 3E6 CAR expressing T cells. Bioluminescent intensity, as shown by increased levels of shading, represents tumor burden.

FIG. 33B presents images. NSG mice were challenged with 1E6 luciferase-expressing leukemia cells as indicated on the figure on day 0. Leukemia in several of these groups were spiked in with 1% of NALM6-CD19neg or NALM6-CD22neg cells. On day 3, mice were treated with 6E6 of CAR expressing T cells. Bioluminescent intensity, as shown by increased levels of shading, represents tumor burden.

FIG. 34A presents images. NSG mice were challenged with 1E6 of NALM6 leukemia on day 0. Mice received treatment with 8E6 mock T, CD19, CD22 or Loop F CAR+ T cells on day 7. Bioluminescent intensity, as shown by increased levels of shading, represents tumor burden.

FIG. 34B presents images. NSG mice were challenged with 1E6 of NALM6 leukemia on day 0. Mice received treatment with 8E6 mock T, CD19, CD22 or Loop F CAR+ T cells on day 7. Bioluminescent intensity, as shown by increased levels of shading, represents tumor burden.

FIGS. 35A and 35B are bar graphs showing IFNγ production of LoopCAR6, post co-incubated with various cell lines representing normal tissues, in accordance with embodiments of the invention.

FIG. 36 is a dot plot showing human PBMC surface expression of the CD19 CAR and CD22 CAR analyzed on day 8.

FIGS. 37A-37G: For cytokine production, CAR T cells (1E5) were washed 3 times with 1XPBS and co-incubated with an equal number of target cells in 200 ml RPMI media in a 96-well plate in a 37° C. incubator for 15 to 20 hours. For high antigen target cells, K562 expression CD19 or CD22 or both CD19 and CD22 were used, and K562 cells served as the negative control. For low target antigen line, NALM6 and the NALM6-CD19^{neg} and NALM6-CD22^{neg} were used, and the NALM6-CD19^{neg} CD22^{neg} was used as the negative control. All tests were in triplicates. The cytokine levels of the IL2 in the culture supernatant were detected with R&D's ELISA kit. FIG. 37A: Cytokine production of CD19 and CD22 CAR with different con-stimulation domains and at different antigen density level. FIG. 37B: Cytokine production of bicistronic CARs with different con-stimulation domains and at different antigen density level. FIG. 37C: Comparison of the cytokine production of bicistronic CAR with that of the bivalent CAR. FIG. 37D-37F: For incucyte killing assay, an equal amount of CAR T cells were co-incubated with 5E4 of target tumor cells. The plate was scanned for the GFP fluorescent expression to monitor the cells every 30 minutes for 40 hours. The percentage of cell killing at each time point was baseline-corrected. FIG. 37G: Incucyte killing assay with NALM6CD19negCD22neg cell.

FIG. 38: RNAseq analysis demonstrates unique gene expression associated with different pairing of costimulatory domain. Bicistronic CAR T cells were co-incubated with an equal number of NALM6 for 24 hours in AMV media. NALM6 cells were removed with magnetic beads, and TRNA was extracted immediately and used for RNAseq analysis. The PCA plot indicates distinct gene expression profiles associated with different pairing of the costimulation domain.

FIGS. 39A and 39B present images. Bioluminescent intensity, as shown by increased levels of shading, represents tumor burden. FIG. 39A: NSG mice were challenged with 1E6 of luciferase-expressing NALM6 leukemia on day 0. On day 3, mice were IV injected with 5E6 of CAR expressing T cells. Bioluminescent intensity represents tumor burden. FIG. 39B: NSG mice were challenged with 1E5 of luciferase-expressing NALM6, NALM6-CD19neg, and NALM6-CD22neg leukemia cells on day 0. On day 3, mice were IV injected with 3E6 of CAR Expressing T cells.

FIG. 40: NSG mice were challenged with 2.5E5 of luciferase-expressing NALM6-CD19^{neg}, and NALM6-CD22^{neg} leukemia cells on day 0. On day 3, mice were IV injected with 3E6 of CAR expressing T cells. Bioluminescent intensity, as shown by increased levels of shading, represents tumor burden.

FIG. 41: NSG mice were IV injected with luciferase-expressing HMB28 Patient derived ALL xenograft (CD19^{neg} CD22+, 1×10⁶). On day 8, mice were injected with 3E6 CAR expressing T cells as indicated in the figure. Bioluminescent intensity, as shown by increased levels of shading, represents tumor burden.

FIG. 42 is a graph showing site density, as described in Example 7.

### DETAILED DESCRIPTION OF THE INVENTION

An embodiment of the invention provides a chimeric antigen receptor (CAR) amino acid construct comprising (a) a cleavable domain; (b) a first CAR comprising a first antigen binding domain, a first transmembrane domain, and a first intracellular T cell signaling domain; and (c) a second CAR comprising a second antigen binding domain, a second transmembrane domain, and a second intracellular T cell signaling domain; wherein the first and second CARs are linked through the cleavable domain, wherein the first antigen binding domain comprises an antigen binding domain of the m971 antibody, wherein when the first CAR is cleaved from the construct, the first antigen binding domain has antigenic specificity for CD22.

A CAR is an artificially constructed hybrid protein or polypeptide containing the antigen binding domain of one or more antibodies (e.g., single chain variable fragment (scFv)) linked to T-cell signaling domains. Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains. The phrases “antigen(ic) specificity” and “elicit antigen-specific response,” as used herein, means that the CAR can specifically bind to and immunologically recognize antigen, such that binding of the CAR to the antigen elicits an immune response.

CD22 is a lineage-restricted B cell antigen belonging to the immunoglobulin (Ig) superfamily. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells (Vaickus et al., Crit. Rev. Oncol. Hematol., 11:267-297 (1991); Bang et al., Clin. Cancer Res., 11:1545-50 (2005)). CD19 (also known as B-lymphocyte antigen CD19, B4, and CVID3) is a cell surface molecule expressed only by B lymphocytes and follicular dendritic cells of the hematopoietic system. It is the earliest of the B-lineage-restricted antigens to be expressed and is present on most pre-B-cells and most non-T-cell acute lymphocytic leukemia cells and B-cell type chronic lymphocytic leukemia cells (Tedder and Isaacs, J. Immun., 143:712-717 (1989)).

In embodiments of the invention, the invention provides multiple CARs (e.g., two, three, four, five, or more) that each bind to a single antigen, wherein each CAR is separated by a cleavable domain. In an embodiment of the invention, cleaving the cleavable domain releases each CAR, e.g., a first and second CAR, from the CAR construct such that each cleaved CAR is separately present on the T cell surface, each has antigenic specificity for its respective target, and each can elicit an antigen-specific response. In an embodiment, such a CAR construct can have two CARs cleaved/released, e.g., a bicistronic CAR. Without wishing to be bound by theory or mechanism, the cleavable domains of these CARs may be cleaved after full translation of the full sequence or after translation of each CAR and cleavable domain, such that a CAR is cleaved/released prior to translation of the next CAR in the sequence. Examples of such CARs herein include V1, V5, V6, V7, and V8.

In embodiments of the invention, the invention provides CARs, where each CAR can bind to two antigens (e.g., CD19 and CD22) simultaneously. These CARs have dual specificity for CD22 and CD19. The phrases “dual specificity,” “dual specific,” “bispecific,” and “bivalent” as used herein with respect to a CAR, mean that the same CAR can specifically bind to and immunologically recognize two different antigens, such that binding of the CAR to at least one of the two antigens elicits an immune response. Examples of such CARs herein include TanCARs 2-4 and LoopCARs 1-5. In another embodiment, dual specific CARs may be linked by cleavable domains.

An embodiment of the invention provides a CAR comprising an anti-CD22 antigen binding domain of the m971 antibody (“m971”). The antigen binding domain of m971 specifically binds to CD22. In this regard, a preferred embodiment of the invention provides CARs comprising an anti-CD22 antigen-binding domain comprising, consisting of, or consisting essentially of, a single chain variable fragment (scFv) of the antigen binding domain of m971. The HA22 immunotoxin and the m971 antibody bind to different CD22 epitopes.

The anti-CD22 antigen binding domain may comprise a light chain variable region and/or a heavy chain variable region. In an embodiment of the invention, the heavy chain variable region comprises a CDR1 region, a CDR2 region, and a CDR3 region. In this regard, the anti-CD22 antigen binding domain may comprise one or more of a heavy chain CDR1 region comprising the amino acid sequence of SEQ ID NO: 4; a heavy chain CDR2 region comprising the amino acid sequence of SEQ ID NO: 6; and a heavy chain CDR3 region comprising the amino acid sequence of SEQ ID NO: 8. Preferably, the heavy chain of the anti-CD22 antigen binding domain comprises the amino acid sequences of all of SEQ ID NOs: 4, 6, and 8.

In an embodiment of the invention, the light chain variable region of the anti-CD22 antigen binding domain may comprise a light chain CDR1 region, a light chain CDR2 region, and a light chain CDR3 region. In this regard, the anti-CD22 antigen binding domain may comprise one or more of a light chain CDR1 region comprising the amino acid sequence of SEQ ID NO: 12; a light chain CDR2 region comprising the amino acid sequence of SEQ ID NO: 14; and a light chain CDR3 region comprising the amino acid sequence of SEQ ID NO: 16. Preferably, the light chain of the anti-CD22 antigen binding domain comprises the amino acid sequences of all of SEQ ID NOs: 12, 14, and 16. In an especially preferred embodiment, the anti-CD22 antigen binding domain comprises the amino acid sequences of all of SEQ ID NO: 4, 6, 8, 12, 14, and 16.

The heavy chain variable region of the anti-CD22 antigen binding domain may comprise, consist of, or consist essentially of the amino acid sequence of SEQ ID NO: 3-9. The light chain variable region of the anti-CD22 antigen binding domain may comprise, consist of, or consist essentially of the amino acid sequence of SEQ ID NO: 11-17. Accordingly, in an embodiment of the invention, the anti-CD22 antigen binding domain comprises a heavy chain variable region comprising the amino acid sequence of SEQ ID NO: 3-9 and/or a light chain variable region comprising the amino acid sequence of SEQ ID NO: 11-17. Preferably, the anti-CD22 antigen binding domain comprises the amino acid sequence of SEQ ID NOs: 3-9 and 11-17.

In embodiments of the invention, when the second CAR is cleaved from the construct, the second antigen binding domain has antigenic specificity for CD19.

In embodiments of the invention, the second antigen binding domain comprises an antigen binding domain of the FMC63 antibody. In embodiments of the invention, the second antigen binding domain comprises a heavy chain variable region comprising the amino acid sequence of FMC63 as described below and a light chain variable region comprising the amino acid sequence of FMC63 as described below. In embodiments of the invention, the second antigen binding domain comprises the amino acid sequence of FMC63 as described below.

Another embodiment of the invention provides a chimeric antigen receptor (CAR) amino acid construct comprising (a) a cleavable domain; (b) a first CAR comprising a first antigen binding domain, a first transmembrane domain, and a first intracellular T cell signaling domain; and (c) a second CAR comprising a second antigen binding domain, a second transmembrane domain, and a second intracellular T cell signaling domain; wherein the first and second CARs are linked through the cleavable domain, wherein the first antigen binding domain comprises an antigen binding domain of the FMC63 antibody, wherein when the first CAR is cleaved from the construct, the first antigen binding domain has antigenic specificity for CD19. In embodiments, when the second CAR is cleaved from the construct, the second antigen binding domain has antigenic specificity for CD22.

An embodiment of the invention provides a CAR comprising an anti-CD19 antigen binding domain of the FMC63 antibody (“FMC63”). The antigen binding domain of FMC63 specifically binds to CD19. In this regard, a preferred embodiment of the invention provides CARs comprising an anti-CD19 antigen-binding domain comprising, consisting of, or consisting essentially of, a single chain variable fragment (scFv) of the antigen binding domain of FMC63.

The anti-CD19 antigen binding domain may comprise a light chain variable region and/or a heavy chain variable region.

In an embodiment of the invention, the light chain variable region of the anti-CD19 antigen binding domain may comprise a light chain CDR1 region, a light chain CDR2 region, and a light chain CDR3 region. In this regard, the anti-CD19 antigen binding domain may comprise one or more of a light chain CDR1 region comprising the amino acid sequence of SEQ ID NO: 24; a light chain CDR2 region comprising the amino acid sequence of SEQ ID NO: 26; and a light chain CDR3 region comprising the amino acid sequence of SEQ ID NO: 28. Preferably, the light chain of the anti-CD19 antigen binding domain comprises the amino acid sequences of all of SEQ ID NOs: 24, 26, and 28.

In an embodiment of the invention, the heavy chain variable region of the anti-CD19 antigen binding domain comprises a CDR1 region, a CDR2 region, and a CDR3 region. In this regard, the anti-CD19 antigen binding domain may comprise one or more of a heavy chain CDR1 region comprising the amino acid sequence of SEQ ID NO: 32; a heavy chain CDR2 region comprising the amino acid sequence of SEQ ID NO: 34; and a heavy chain CDR3 region comprising the amino acid sequence of SEQ ID NO: 36. Preferably, the heavy chain of the anti-CD19 antigen binding domain comprises the amino acid sequences of all of SEQ ID NOs: 32, 34, and 36. In an especially preferred embodiment, the anti-CD19 antigen binding domain comprises the amino acid sequences of all of SEQ ID NO: 24, 26, 28, 32, 34, and 36.

The heavy chain variable region of the anti-CD19 antigen binding domain may comprise, consist of, or consist essentially of the amino acid sequence of SEQ ID NO: 31-37. The light chain variable region of the anti-CD19 antigen binding domain may comprise, consist of, or consist essentially of the amino acid sequence of SEQ ID NO: 23-29. Accordingly, in an embodiment of the invention, the anti-CD19 antigen binding domain comprises a heavy chain variable region comprising the amino acid sequence of SEQ ID NO: 31-37 and/or a light chain variable region comprising the amino acid sequence of SEQ ID NO: 23-29. Preferably, the anti-CD19 antigen binding domain comprises the amino acid sequences of both SEQ ID NOs: 23-29 and 31-37.

The anti-CD22 antigen binding domain and the anti-CD19 antigen binding domain may comprise any antigen binding portion of the anti-CD22 or anti-CD19 antibody, respectively. The antigen binding portion can be any portion that has at least one antigen binding site, such as Fab, F(ab′)₂, dsFv, scFv, diabodies, and triabodies. Preferably, the antigen binding portion is a single-chain variable region fragment (scFv) antibody fragment. An scFv is a truncated Fab fragment including the variable (V) domain of an antibody heavy chain linked to a V domain of a light antibody chain via a synthetic peptide linker, which can be generated using routine recombinant DNA technology techniques. Similarly, disulfide-stabilized variable region fragments (dsFv) can be prepared by recombinant DNA technology.

In an embodiment of the invention, the light chain variable region and the heavy chain variable region of the anti-CD22 antigen binding domain can be joined to each other by a linker. The linker may comprise any suitable amino acid sequence. In an embodiment of the invention, the linker is a Gly/Ser linker from about 1 to about 100, from about 3 to about 20, from about 5 to about 30, from about 5 to about 18, or from about 3 to about 8 amino acids in length and consists of glycine and/or serine residues in sequence. Accordingly, the Gly/Ser linker may consist of glycine and/or serine residues. Preferably, the Gly/Ser linker comprises the amino acid sequence of GGGGS (SEQ ID NO: 10), and multiple SEQ ID NOs: 10 may be present within the linker. In another embodiment of the invention, the linker comprises the amino acid sequence of SEQ ID NO: 30. Any linker sequence may be used as a spacer between the antigen binding domain and the transmembrane domain.

In an embodiment of the invention, the light chain variable region and the heavy chain variable region of the anti-CD19 antigen binding domain can be joined to each other by a linker. The linker may be any of the linkers described herein with respect to other aspects of the invention. In an embodiment of the invention, the light chain variable region and the heavy chain variable region of the anti-CD19 antigen binding domain are joined to each other by a linker comprising the amino acid sequence of SEQ ID NO: 10 or 30.

In an embodiment, the anti-CD22 antigen binding domain comprises a light chain variable region, a heavy chain variable region, and a linker. In this regard, an embodiment of the anti-CD22 antigen binding domain comprising a light chain variable region, a heavy chain variable region, and the linker comprises, consists of, or consists essentially of, all of SEQ ID NOs: 3-17.

In an embodiment, the anti-CD19 antigen binding domain comprises a light chain variable region, a heavy chain variable region, and a linker. In this regard, an embodiment of the anti-CD19 antigen binding domain comprising a light chain variable region, a heavy chain variable region, and the linker comprises, consists of, or consists essentially of, all of SEQ ID NOs: 23-37.

The first CAR and the second CAR of the inventive CAR constructs are joined to each other through 1, 2, 3, 4 or more cleavable domains. The cleavable domain(s) may comprise one or more of any suitable cleavable domain, including domains recognized by cleavage enzymes or domains that are self-cleaving. Suitable domains include, for example, the 2A domain, such as T2A and/or P2A, and furin cleavage sequences. Table 1 presents exemplary suitable cleavable domains.

TABLE 1

| Peptide | Amino acid sequence* |
|---|---|
| T2A: | (GSG)EGRGSLLTCGDVEENPGP |
| | (SEQ ID NO: 43) |
| P2A: | (GSG)ATNFSLLKQAGDVEENPGP |
| | (SEQ ID NO: 44) |
| E2A: | (GSG)QCTNYALLKLAGDVESNPGP |
| | (SEQ ID NO: 45) |
| F2A: | (GSG)VKQTLNFDLLKLAGDVESNPGP |
| | (SEQ ID NO: 46) |
| Furin: | RKRR |
| | (SEQ ID NO: 47) |

*The GSG residues may be added to improve cleavage efficiency.

In an embodiment of the invention, the CAR construct contains more than one cleavable domain, wherein the cleavable domains are all the same. In an embodiment of the invention, the CAR construct contains more than one cleavable domain adjacent within the CAR construct, wherein at least one cleavable domain is different.

In an embodiment, the antigen binding domain comprises a leader sequence. In an embodiment of the invention, the leader sequence may be positioned at the amino terminus of the anti-CD19 CAR within the CAR construct. In another embodiment of the invention, the leader sequence may be positioned at the amino terminus of the anti-CD22 CAR within the CAR construct. The leader sequence may comprise any suitable leader sequence. In an embodiment, the leader sequence comprises, consists of, or consists essentially of the amino acid sequence of SEQ ID NO: 2 or SEQ ID NO: 62. In an embodiment of the invention, while the leader sequence may facilitate expression of the released/cleaved CARs on the surface of the cell, the presence of the leader sequence in an expressed CAR is not necessary in order for the CAR to function. In an embodiment of the invention, upon expression of the CAR on the cell surface, the leader sequence may be cleaved off released CARs. Accordingly, in an embodiment of the invention, the released CARs lack a leader sequence. In an embodiment of the invention, the CARs within the CAR construct lack a leader sequence.

In an embodiment of the invention, the CAR construct comprises a hinge domain. In an embodiment of the invention, the hinge domain is a CD8 hinge domain. In a preferred embodiment, the CD8 hinge domain is human. Preferably, the CD8 hinge domain comprises, consists of, or consists essentially of SEQ ID NO: 18. In an embodiment of the invention, the hinge domain is a CD28 hinge domain. In a preferred embodiment, the CD28 hinge domain is human. Preferably, the CD28 hinge domain comprises, consists of, or consists essentially of SEQ ID NO: 40.

In an embodiment of the invention, the CAR construct comprises a transmembrane (TM) domain. In an embodiment of the invention, the TM domain is a CD8 TM domain. In a preferred embodiment, the CD8 TM domain is human. Preferably, the CD8 TM domain comprises, consists of, or consists essentially of SEQ ID NO: 19. In an embodiment of the invention, the TM domain is a CD28 TM domain. In a preferred embodiment, the CD28 TM domain is human. Preferably, the CD28 TM domain comprises, consists of, or consists essentially of SEQ ID NO: 41.

In an embodiment of the invention, the CAR construct comprises an intracellular T cell signaling domain. In an embodiment of the invention, the intracellular T cell signaling domain comprises a 4-1BB intracellular T cell signaling sequence. 4-1BB, also known as CD137, transmits a potent costimulatory signal to T cells, promoting differentiation and enhancing long-term survival of T lymphocytes. Preferably, the 4-1BB intracellular T cell signaling sequence is human. In a preferred embodiment, the 4-1BB intracellular T cell signaling sequence comprises, consists of, or consists essentially of the amino acid sequence of SEQ ID NO: 20.

In an embodiment of the invention, the intracellular T cell signaling domain comprises a CD3 zeta (ζ) intracellular T cell signaling sequence. CD3ζ associates with TCRs to produce a signal and contains immunoreceptor tyrosine-based activation motifs (ITAMs). Preferably, the CD3ζ intracellular T cell signaling sequence is human. In a preferred embodiment, the CD3ζ intracellular T cell signaling sequence comprises, consists of, or consists essentially of the amino acid sequence of SEQ ID NO: 21.

In an embodiment of the invention, the intracellular T cell signaling domain comprises a CD28 intracellular T cell signaling sequence. Preferably, the CD28 intracellular T cell signaling sequence is human. In a preferred embodiment, the CD28 intracellular T cell signaling sequence comprises, consists of, or consists essentially of the amino acid sequence of SEQ ID NO: 42.

The first and second CARs may be positioned in the CAR construct in any suitable orientation. In an embodiment of the invention, the CAR construct comprises from the N- to C-terminus: the anti-CD19 CAR, one or more cleavable domains, and then the anti-CD22 CAR. In another embodiment of the invention, the CAR construct comprises from the N- to C-terminus: the anti-CD22 CAR, one or more cleavable domains, and then the anti-CD19 CAR.

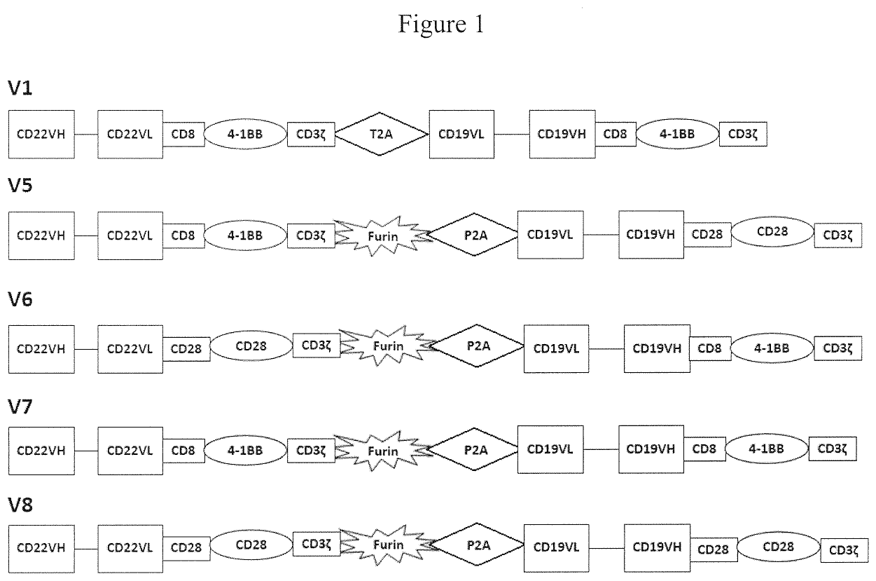

FIG. 1 presents schematic diagrams of exemplary CAR constructs, in accordance with embodiments of the invention.

Additional embodiments of the invention provide full-length CAR constructs comprising, consisting of, or consisting essentially of, any one of the amino acid sequences set forth in Tables 2-6.

TABLE 2
V1 CAR Construct

| | SEQ ID | | |
|---|---|---|---|
| Sequence | NO: | Segment | Notes |
| M | 1 | | start methionine |
| LLLVTSLLLCELPHPAFLLIP | 2 | | signal peptide |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GGGGS | 10 | Anti-CD22 scFv | linker |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGRAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| TSTTTPAPRPPTPAPTIASQPL | 18 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |
| GSGEGRGSLLTCGDVEENPG | 22 | | T2A cleavable domain |
| PR | | | |
| M | 1 | | start methionine |
| LLLVTSLLLCELPHPAFLLIP | 2 | | signal peptide |
| DIQMTQTTSSLSASLGDRVTI | 23 | Anti-CD19 scFv | light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GSTSGSGKPGSGEGSTKG | 30 | Anti-CD19 scFv | linker |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

TABLE 3
V5 CAR Construct

| | SEQ ID | | |
|---|---|---|---|
| Sequence | NO: | Segment | Notes |
| M | 1 | | start methionine |
| ALPVTALLLPLALLLHAARP | 62 | | signal peptide |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GGGGS | 10 | Anti-CD22 scFv | linker |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |
| RKRRGSGTPDPW | 38 | | Furin cleavable domain |
| GSGATNFSLLKQAGDVEENP | 39 | | P2A cleavable domain |
| GPLE | | | |
| M | 1 | | start methionine |
| EFGLSWLFLVAILKGVQCSR | 87 | | signal peptide |
| DIQMTQTTSSLSASLGDRVTI | 23 | Anti-CD19 scFv | light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GSTSGSGKPGSGEGSTKG | 30 | Anti-CD19 scFv | linker |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| TSAAAIEVMYPPPYLDNEKS | 40 | CD28 hinge | Extracellular |
| NGTIIHVKGKHLCPSPLFPGP | | | |
| SKP | | | |
| FWVLVVVGGVLACYSLLVT | 41 | CD28 | transmembrane domain |
| VAFIIFWV | | | |
| RSKRSRLLHSDYMNMTPRRP | 42 | CD28 | intracellular domain |
| GPTRKHYQPYAPPRDFAAYR | | | |
| S | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

TABLE 4
V6 CAR Construct

| | SEQ ID | | |
|---|---|---|---|
| Sequence | NO: | Segment | Notes |
| M | 1 | | start methionine |
| ALPVTALLLPLALLLHAARP | 62 | | signal peptide |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GGGGS | 10 | Anti-CD22 scFv | linker |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| SGAAAIEVMYPPPYLDNEKS | 40 | CD28 hinge | Extracellular |
| NGTIIHVKGKHLCPSPLFPGP | | | |
| SKP | | | |
| FWVLVVVGGVLACYSLLVT | 41 | CD28 | transmembrane domain |
| VAFIIFWV | | | |
| RSKRSRLLHSDYMNMTPRRP | 42 | CD28 | intracellular domain |
| GPTRKHYQPYAPPRDFAAYR | | | |
| S | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |
| RKRRGSGTPDPW | 38 | | Furin cleavable domain |
| GSGATNFSLLKQAGDVEENP | 39 | | P2A cleavable domain |
| GPLE | | | |
| M | 1 | | start methionine |
| EFGLSWLFLVAILKGVQCSR | 87 | | signal peptide |
| DIQMTQTTSSLSASLGDRVTI | 23 | Anti-CD19 scFv | light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GSTSGSGKPGSGEGSTKG | 30 | Anti-CD19 scFv | linker |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| TSTTTPAPRPPTPAPTIASQPL | 18 | CD8 | CD8 alpha hinge |
| SLRPEACRPAAGGAVHTRGL | | | |
| DFACD | | | |
| IYIWAPLAGTCGVLLLSLVIT | 19 | CD8 | CD8 alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

TABLE 5
V7 CAR Construct

| | SEQ ID | | |
|---|---|---|---|
| Sequence | NO: | Segment | Notes |
| M | 1 | | start methionine |
| ALPVTALLLPLALLLHAARP | 62 | | signal peptide |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GGGGS | 10 | Anti-CD22 scFv | linker |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |
| RKRRGSGTPDPW | 38 | | Furin cleavable domain |
| GSGATNFSLLKQAGDVEENP | 39 | | P2A cleavable domain |
| GPLE | | | |
| M | 1 | | start methionine |
| EFGLSWLFLVAILKGVQCSR | 87 | | signal peptide |
| DIQMTQTTSSLSASLGDRVTI | 23 | Anti-CD19 scFv | light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GSTSGSGKPGSGEGSTKG | 30 | Anti-CD19 scFv | linker |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| TSTTTPAPRPPTPAPTIASQPL | 18 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

TABLE 6
V8 CAR Construct

| | SEQ ID | | |
|---|---|---|---|
| Sequence | NO: | Segment | Notes |
| M | 1 | | start methionine |
| ALPVTALLLPLALLLHAARP | 62 | | signal peptide |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GGGGS | 10 | Anti-CD22 scFv | linker |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| SGAAAIEVMYPPPYLDNEKS | 40 | CD28 | CD28 hinge |
| NGTIIHVKGKHLCPSPLFPGP | | | |
| SKP | | | |
| FWVLVVVGGVLACYSLLVT | 41 | CD28 | transmembrane domain |
| VAFIIFWV | | | |
| RSKRSRLLHSDYMNMTPRRP | 42 | CD28 | intracellular domain |
| GPTRKHYQPYAPPRDFAAYR | | | |
| S | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |
| RKRRGSGTPDPW | 38 | | Furin cleavable domain |
| GSGATNFSLLKQAGDVEENP | 39 | | P2A cleavable domain |
| GPLE | | | |
| M | 1 | | start methionine |
| EFGLSWLFLVAILKGVQCSR | 87 | | signal peptide |
| DIQMTQTTSSLSASLGDRVTI | 23 | Anti-CD19 scFv | light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GSTSGSGKPGSGEGSTKG | 30 | Anti-CD19 scFv | linker |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| TSAAAIEVMYPPPYLDNEKS | 40 | CD28 | CD28 hinge |
| NGTIIHVKGKHLCPSPLFPGP | | | |
| SKP | | | |
| FWVLVVVGGVLACYSLLVT | 41 | CD28 | transmembrane domain |
| VAFIIFWV | | | |
| RSKRSRLLHSDYMNMTPRRP | 42 | CD28 | intracellular domain |
| GPTRKHYQPYAPPRDFAAYR | | | |
| S | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR | | | |

TABLE 7
TanCAR 1 Construct

| | SEQ ID | | |
|---|---|---|---|
| Sequence | NO: | Segment | Notes |
| M | 1 | | start methionine |
| LLLVTSLLLCELPHPAFLLIP | 2 | | signal peptide |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GGGGS | 10 | Anti-CD22 scFv | Linker 1 |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| GGGGSGGGGSGGGGSGGGG | 79 | Linker | Linker 5 |
| SGGGGS | | | |
| DIQMTQTTSSLSASLGDRVTI | 23 | Anti-CD19 scFv | Light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GSTSGSGKPGSGEGSTKG | 30 | Anti-CD19 scFv | Linker 6 |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR* | | | |

TABLE 8
TanCAR 2 Construct

| | SEQ ID | | |
|---|---|---|---|
| Sequence | NO: | Segment | Notes |
| M | 1 | | start methionine |
| LLLVTSLLLCELPHPAFLLIP | 2 | | signal peptide |
| DIQMTQTTSSLSASLGDRVTI | 23 | Anti-CD19 scFv | Light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GSTSGSGKPGSGEGSTKG | 30 | Anti-CD19 scFv | Linker 6 |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| GGGGSGGGGSGGGGSGGGG | 79 | Linker | Linker 5 |
| SGGGGS | | | |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GGGGS | 10 | Anti-CD22 scFv | Linker 1 |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR* | | | |

TABLE 9
TanCAR 3 Construct

| | SEQ ID | | |
|---|---|---|---|
| Sequence | NO: | Segment | Notes |
| M | 1 | | start methionine |
| LLLVTSLLLCELPHPAFLLIP | 2 | | signal peptide |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GSTSGSGKPGSGEGSTKG | 30 | Anti-CD22 scFv | Linker 6 |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| GGGGSGGGGSGGGGSGGGG | 79 | Linker | Linker 5 |
| SGGGGS | | | |
| DIQMTQTTSSLSASLGDRVTI | 23 | Anti-CD19 scFv | Light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GSTSGSGKPGSGEGSTKG | 30 | Anti-CD19 scFv | Linker 6 |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR* | | | |

TABLE 10
TanCAR 4 Construct

| | SEQ ID | | |
|---|---|---|---|
| Sequence | NO: | Segment | Notes |
| M | 1 | | start methionine |
| ALPVTALLLPLALLLHAARP | 62 | | signal peptide |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GGGGS | 10 | Anti-CD22 scFv | Linker 1 |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| GGGGSGGGGSGGGGSGGGG | 88 | Linker | Linker 4 |
| S | | | |
| DIQMTQTTSSLSASLGDRVTI | 23 | Anti-CD19 scFv | Light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GSTSGSGKPGSGEGSTKG | 30 | Anti-CD19 scFv | Linker 6 |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR* | | | |

TABLE 11
Loop CAR 1 Construct

| | SEQ ID | | |
|---|---|---|---|
| Sequence | NO: | Segment | Notes |
| M | 1 | | start methionine |
| LLLVTSLLLCELPHPAFLLIP | 2 | | signal peptide |
| DIQMTQTTSSLSASLGDRVTI | 23 | Anti-CD19 scFv | Light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GGGGSGGGGSGGGGS | 80 | Linker | Linker 3 |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GGGGS | 10 | Anti-CD22 scFv | Linker 1 |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| GGGGSGGGGSGGGGS | 80 | Anti-CD22 scFv | Linker 3 |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR* | | | |

TABLE 12
Loop CAR 2 Construct

| | SEQ ID | | |
|---|---|---|---|
| Sequence | NO: | Segment | Notes |
| M | 1 | | start methionine |
| LLLVTSLLLCELPHPAFLLIP | 2 | | signal peptide |
| DIQMTQTTSSLSASLGDRVTI | 23 | Anti-CD19 scFv | Light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GGCGSGGGGSGGGGS | 81 | Linker | Linker 3A |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GSTSGSGKPGSGEGSTKG | 30 | Anti-CD22 scFv | Linker 6 |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| GGGGSGGGGSGGCGS | 82 | Anti-CD22 scFv | Linker 3B |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR* | | | |

TABLE 13
Loop CAR 3 Construct

| | SEQ ID | | |
|---|---|---|---|
| Sequence | NO: | Segment | Notes |
| M | 1 | | start methionine |
| ALPVTALLLPLALLLHAARP | 62 | | signal peptide |
| DIQMTQTTSSLSASLGDRVTI | 23 | Anti-CD19 scFv | Light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GGGGSGGGGS | 83 | Linker | Linker 2 |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GSTSGSGKPGSGEGSTKG | 30 | Anti-CD22 scFv | Linker 6 |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| GGGGSGGGGS | 83 | Linker | Linker 2 |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3 zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR* | | | |

TABLE 14
Loop CAR 4 Construct

| | SEQ ID | | |
|---|---|---|---|
| Sequence | NO: | Segment | Notes |
| M | 1 | | start methionine |
| LLLVTSLLLCELPHPAFLLIP | 2 | | signal peptide |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GGGGSGGGGS | 83 | Linker | Linker 2 |
| DIQMTQTTSSLSASLGDRVTI | 23 | Anti-CD19 scFv | Light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GSTSGSGKPGSGEGSTKG | 30 | Anti-CD19 scFv | Linker 6 |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| GGGGSGGGGS | 83 | Linker | Linker 2 |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR* | | | |

TABLE 15
Loop CAR 5 Construct

| | SEQ ID | | |
|---|---|---|---|
| Sequence | NO: | Segment | Notes |
| M | 1 | | start methionine |
| LLLVTSLLLCELPHPAFLLIP | 2 | | signal peptide |
| DIQMTQTTSSLSASLGDRVTI | 23 | Anti-CD19 scFv | Light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GGGGSGGGGSCGGGS | 84 | Linker | Linker 3C |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GSTSGSGKPGSGEGSTKG | 30 | Anti-CD22 scFv | Linker 6 |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| GGGGCGGGGSGGGGS | 85 | Linker | Linker 3D |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR* | | | |

TABLE 16
Loop CAR 6 Construct

| | SEQ ID | | |
|---|---|---|---|
| Sequence | NO: | Segment | Notes |
| M | 1 | | start methionine |
| LLLVTSLLLCELPHPAFLLIP | 2 | | signal peptide |
| DIQMTQTTSSLSASLGDRVTI | 23 | Anti-CD19 scFv | Light chain |
| SCRAS | | | |
| QDISKY | 24 | Anti-CD19 scFv | light chain: CDR1 |
| LNWYQQKPDGTVKLLIY | 25 | Anti-CD19 scFv | light chain |
| HTS | 26 | Anti-CD19 scFv | light chain: CDR2 |
| RLHSGVPSRFSGSGSGTDYS | 27 | Anti-CD19 scFv | light chain |
| LTISNLEQEDIATYFC | | | |
| QQGNTLPYT | 28 | Anti-CD19 scFv | light chain: CDR3 |
| FGGGTKLEIT | 29 | Anti-CD19 scFv | light chain |
| GGGGS | 10 | Linker | Linker 1 |
| QVQLQQSGPGLVKPSQTLSL | 3 | Anti-CD22 scFv | heavy chain |
| TCAIS | | | |
| GDSVSSNSAA | 4 | Anti-CD22 scFv | heavy chain: CDR1 |
| WNWIRQSPSRGLEWLGR | 5 | Anti-CD22 scFv | heavy chain |
| TYYRSKWYN | 6 | Anti-CD22 scFv | heavy chain: CDR2 |
| DYAVSVKSRITINPDTSKNQF | 7 | Anti-CD22 scFv | heavy chain |
| SLQLNSVTPEDTAVYYC | | | |
| AREVTGDLEDAFDI | 8 | Anti-CD22 scFv | heavy chain: CDR3 |
| WGQGTMVTVSS | 9 | Anti-CD22 scFv | heavy chain |
| GSTSGSGKPGSGEGSTKG | 30 | Anti-CD22 scFv | Linker 6 |
| DIQMTQSPSSLSASVGDRVTI | 11 | Anti-CD22 scFv | light chain |
| TCRAS | | | |
| QTIWSY | 12 | Anti-CD22 scFv | light chain: CDR1 |
| LNWYQQRPGKAPNLLIY | 13 | Anti-CD22 scFv | light chain |
| AAS | 14 | Anti-CD22 scFv | light chain: CDR2 |
| SLQSGVPSRFSGRGSGTDFTL | 15 | Anti-CD22 scFv | light chain |
| TISSLQAEDFATYYC | | | |
| QQSYSIPQT | 16 | Anti-CD22 scFv | light chain: CDR3 |
| FGQGTKLEIK | 17 | Anti-CD22 scFv | light chain |
| GGGGS | 10 | Linker | Linker 1 |
| EVKLQESGPGLVAPSQSLSV | 31 | Anti-CD19 scFv | heavy chain |
| TCTVS | | | |
| GVSLPDYG | 32 | Anti-CD19 scFv | heavy chain: CDR1 |
| VSWIRQPPRKGLEWLGV | 33 | Anti-CD19 scFv | heavy chain |
| IWGSETT | 34 | Anti-CD19 scFv | heavy chain: CDR2 |
| YYNSALKSRLTIIKDNSKSQV | 35 | Anti-CD19 scFv | heavy chain |
| FLKMNSLQTDDTAIYYC | | | |
| AKHYYYGGSYAMDY | 36 | Anti-CD19 scFv | heavy chain: CDR3 |
| WGQGTSVTVSS | 37 | Anti-CD19 scFv | heavy chain |
| SGTTTPAPRPPTPAPTIASQPL | 86 | CD8 | CD8alpha hinge |
| SLRPEACRPAAGGAVHTRGL | | | |
| DFACD | | | |
| IYIWAPLAGTCGVLLLSLVIT | 19 | CD8 | CD8alpha transmembrane |
| LYC | | | domain |
| KRGRKKLLYIFKQPFMRPVQ | 20 | 4-1BB | intracellular domain |
| TTQEEDGCSCRFPEEEEGGC | | | |
| EL | | | |
| RVKFSRSADAPAYQQGQNQ | 21 | CD3zeta | intracellular domain |
| LYNELNLGRREEYDVLDKR | | | |
| RGRDPEMGGKPRRKNPQEG | | | |
| LYNELQKDKMAEAYSEIGM | | | |
| KGERRRGKGHDGLYQGLST | | | |
| ATKDTYDALHMQALPPR* | | | |

In an embodiment, the CAR construct (herein denoted V1) has the sequence:

(SEQ ID NO: 48)
MLLLVTSLLLCELPHPAFLLIPQVQLQQSGPGLVKPSQTLSLTCAISGDS
VSSNSAAWNWIRQSPSRGLEWLGRTYYRSKWYNDYAVSVKSRITINPDTS
KNQFSLQLNSVTPEDTAVYYCAREVTGDLEDAFDIWGQGTMVTVSSGGGG
SDIQMTQSPSSLSASVGDRVTITCRASQTIWSYLNWYQQRPGKAPNLLIY
AASSLQSGVPSRFSGRGSGTDFTLTISSLQAEDFATYYCQQSYSIPQTFG
QGTKLEIKTSTTTPAPRPPTPAPTIASQPLSLRPEACRPAAGGAVHTRGL
DFACDIYIWAPLAGTCGVLLLSLVITLYCKRGRKKLLYIFKQPFMRPVQT
TQEEDGCSCRFPEEEEGGCELRVKFSRSADAPAYQQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPRGSGEGRGSLLTCGDVEE
NPGPRMLLLVTSLLLCELPHPAFLLIPDIQMTQTTSSLSASLGDRVTISC
RASQDISKYLNWYQQKPDGTVKLLIYHTSRLHSGVPSRFSGSGSGTDYSL
TISNLEQEDIATYFCQQGNTLPYTFGGGTKLEITGSTSGSGKPGSGEGST
KGEVKLQESGPGLVAPSQSLSVTCTVSGVSLPDYGVSWIRQPPRKGLEWL
GVIWGSETTYYNSALKSRLTIIKDNSKSQVFLKMNSLQTDDTAIYYCAKH
YYYGGSYAMDYWGQGTSVTVSSSGTTTPAPRPPTPAPTIASQPLSLRPEA
CRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYQQ
GQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR.

In an embodiment, the CAR construct (herein denoted V5) has the sequence:

(SEQ ID NO: 49)
MALPVTALLLPLALLLHAARPQVQLQQSGPGLVKPSQTLSLTCAISGDSV
SSNSAAWNWIRQSPSRGLEWLGRTYYRSKWYNDYAVSVKSRITINPDTSK
NQFSLQLNSVTPEDTAVYYCAREVTGDLEDAFDIWGQGTMVTVSSGGGGS
DIQMTQSPSSLSASVGDRVTITCRASQTIWSYLNWYQQRPGKAPNLLIYA
ASSLQSGVPSRFSGRGSGTDFTLTISSLQAEDFATYYCQQSYSIPQTFGQ
GTKLEIKSGTTTPAPRPPTPAPTIASQPLSLRPEACRPAAGGAVHTRGLD
FACDIYIWAPLAGTCGVLLLSLVITLYCKRGRKKLLYIFKQPFMRPVQTT
QEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPRRKRRGSGTPDPWGSGATN
FSLLKQAGDVEENPGPLEMEFGLSWLFLVAILKGVQCSRDIQMTQTTSSL
SASLGDRVTISCRASQDISKYLNWYQQKPDGTVKLLIYHTSRLHSGVPSR
FSGSGSGTDYSLTISNLEQEDIATYFCQQGNTLPYTFGGGTKLEITGSTS
GSGKPGSGEGSTKGEVKLQESGPGLVAPSQSLSVTCTVSGVSLPDYGVSW
IRQPPRKGLEWLGVIWGSETTYYNSALKSRLTIIKDNSKSQVFLKMNSLQ
TDDTAIYYCAKHYYYGGSYAMDYWGQGTSVTVSSTSAAAIEVMYPPPYLD
NEKSNGTIIHVKGKHLCPSPLFPGPSKPFWVLVVVGGVLACYSLLVTVAF
IIFWVRSKRSRLLHSDYMNMTPRRPGPTRKHYQPYAPPRDFAAYRSRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPR.

In an embodiment, the CAR construct (herein denoted V6) has the sequence:

(SEQ ID NO: 50)
MALPVTALLLPLALLLHAARPQVQLQQSGPGLVKPSQTLSLTCAISGDSV
SSNSAAWNWIRQSPSRGLEWLGRTYYRSKWYNDYAVSVKSRITINPDTSK
NQFSLQLNSVTPEDTAVYYCAREVTGDLEDAFDIWGQGTMVTVSSGGGGS
DIQMTQSPSSLSASVGDRVTITCRASQTIWSYLNWYQQRPGKAPNLLIYA
ASSLQSGVPSRFSGRGSGTDFTLTISSLQAEDFATYYCQQSYSIPQTFGQ
GTKLEIKSGAAAIEVMYPPPYLDNEKSNGTIIHVKGKHLCPSPLFPGPSK
PFWVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRREE
YDVLDKRRGRDPEMGGKPRRKNPQEGLYNELQKDKMAEAYSEIGMKGERR
RGKGHDGLYQGLSTATKDTYDALHMQALPPRRKRRGSGTPDPWGSGATNF
SLLKQAGDVEENPGPLEMEFGLSWLFLVAILKGVQCSRDIQMTQTTSSLS
ASLGDRVTISCRASQDISKYLNWYQQKPDGTVKLLIYHTSRLHSGVPSRF
SGSGSGTDYSLTISNLEQEDIATYFCQQGNTLPYTFGGGTKLEITGSTSG
SGKPGSGEGSTKGEVKLQESGPGLVAPSQSLSVTCTVSGVSLPDYGVSWI
RQPPRKGLEWLGVIWGSETTYYNSALKSRLTIIKDNSKSQVFLKMNSLQT
DDTAIYYCAKHYYYGGSYAMDYWGQGTSVTVSSTSTTTPAPRPPTPAPTI
ASQPLSLRPEACRPAAGGAVHTRGLDFACDIYIWAPLAGTCGVLLLSLVI
TLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPR.

In an embodiment, the CAR construct (herein denoted V7) has the sequence:

(SEQ ID NO: 51)
MALPVTALLLPLALLLHAARPQVQLQQSGPGLVKPSQTLSLTCAISGDSV
SSNSAAWNWIRQSPSRGLEWLGRTYYRSKWYNDYAVSVKSRITINPDTSK
NQFSLQLNSVTPEDTAVYYCAREVTGDLEDAFDIWGQGTMVTVSSGGGGS
DIQMTQSPSSLSASVGDRVTITCRASQTIWSYLNWYQQRPGKAPNLLIYA
ASSLQSGVPSRFSGRGSGTDFTLTISSLQAEDFATYYCQQSYSIPQTFGQ
GTKLEIKSGTTTPAPRPPTPAPTIASQPLSLRPEACRPAAGGAVHTRGLD
FACDIYIWAPLAGTCGVLLLSLVITLYCKRGRKKLLYIFKQPFMRPVQTT
QEEDGCSCRFPEEEEGGCELRVKFSRSADAPAYQQGQNQLYNELNLGRRE
EYDVLDKRRGRDPEMGGKPRRKNPQEGLYNELQKDKMAEAYSEIGMKGER
RRGKGHDGLYQGLSTATKDTYDALHMQALPPRRKRRGSGTPDPWGSGATN
FSLLKQAGDVEENPGPLEMEFGLSWLFLVAILKGVQCSRDIQMTQTTSSL
SASLGDRVTISCRASQDISKYLNWYQQKPDGTVKLLIYHTSRLHSGVPSR
FSGSGSGTDYSLTISNLEQEDIATYFCQQGNTLPYTFGGGTKLEITGSTS
GSGKPGSGEGSTKGEVKLQESGPGLVAPSQSLSVTCTVSGVSLPDYGVSW
IRQPPRKGLEWLGVIWGSETTYYNSALKSRLTIIKDNSKSQVFLKMNSLQ
TDDTAIYYCAKHYYYGGSYAMDYWGQGTSVTVSSTSTTTPAPRPPTPAPT
IASQPLSLRPEACRPAAGGAVHTRGLDFACDIYIWAPLAGTCGVLLLSLV
ITLYCKRGRKKLLYIFKQPFMRPVQTTQEEDGCSCRFPEEEEGGCELRVK
FSRSADAPAYQQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR.

In an embodiment, the CAR construct (herein denoted V8) has the sequence:

(SEQ ID NO: 52)
MALPVTALLLPLALLLHAARPQVQLQQSGPGLVKPSQTLSLTCAISGDSV
SSNSAAWNWIRQSPSRGLEWLGRTYYRSKWYNDYAVSVKSRITINPDTSK
NQFSLQLNSVTPEDTAVYYCAREVTGDLEDAFDIWGQGTMVTVSSGGGGS
DIQMTQSPSSLSASVGDRVTITCRASQTIWSYLNWYQQRPGKAPNLLIYA
ASSLQSGVPSRFSGRGSGTDFTLTISSLQAEDFATYYCQQSYSIPQTFGQ
GTKLEIKSGAAAIEVMYPPPYLDNEKSNGTIIHVKGKHLCPSPLFPGPSK
PFWVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRREE
YDVLDKRRGRDPEMGGKPRRKNPQEGLYNELQKDKMAEAYSEIGMKGERR
RGKGHDGLYQGLSTATKDTYDALHMQALPPRRKRRGSGTPDPWGSGATNF
SLLKQAGDVEENPGPLEMEFGLSWLFLVAILKGVQCSRDIQMTQTTSSLS
ASLGDRVTISCRASQDISKYLNWYQQKPDGTVKLLIYHTSRLHSGVPSRF
SGSGSGTDYSLTISNLEQEDIATYFCQQGNTLPYTFGGGTKLEITGSTSG
SGKPGSGEGSTKGEVKLQESGPGLVAPSQSLSVTCTVSGVSLPDYGVSWI
RQPPRKGLEWLGVIWGSETTYYNSALKSRLTIIKDNSKSQVFLKMNSLQT
DDTAIYYCAKHYYYGGSYAMDYWGQGTSVTVSSTSAAAIEVMYPPPYLDN
EKSNGTIIHVKGKHLCPSPLFPGPSKPFWVLVVVGGVLACYSLLVTVAFI
IFWVRSKRSRLLHSDYMNMTPRRPGPTRKHYQPYAPPRDFAAYRSRVKFS
RSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALH
MQALPPR.

In an embodiment, the CAR construct (herein denoted TanCAR2) has the sequence:

(SEQ ID NO: 63)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSGGGGSGGGGSGGGGSGGGGSGGGGSQVQLQQSG
PGLVKPSQTLSLTCAISGDSVSSNSAAWNWIRQSPSRGLEWLGRTYYRSK
WYNDYAVSVKSRITINPDTSKNQFSLQLNSVTPEDTAVYYCAREVTGDLE
DAFDIWGQGTMVTVSSGGGGSDIQMTQSPSSLSASVGDRVTITCRASQTI
WSYLNWYQQRPGKAPNLLIYAASSLQSGVPSRFSGRGSGTDFTLTISSLQ
AEDFATYYCQQSYSIPQTFGQGTKLEIKTTTPAPRPPTPAPTIASQPLSL
RPEACRPAAGGAVHTRGLDFACDIYIWAPLAGTCGVLLLSLVITLYCKRG
RKKLLYIFKQPFMRPVQTTQEEDGCSCRFPEEEEGGCELRVKFSRSADAP
AYKQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPP
R.

In an embodiment, the CAR construct (herein denoted TanCAR3) has the sequence:

(SEQ ID NO: 64)
MLLLVTSLLLCELPHPAFLLIPQVQLQQSGPGLVKPSQTLSLTCAISGDS
VSSNSAAWNWIRQSPSRGLEWLGRTYYRSKWYNDYAVSVKSRITINPDTS
KNQFSLQLNSVTPEDTAVYYCAREVTGDLEDAFDIWGQGTMVTVSSGSTS
GSGKPGSGEGSTKGDIQMTQSPSSLSASVGDRVTITCRASQTIWSYLNWY
QQRPGKAPNLLIYAASSLQSGVPSRFSGRGSGTDFTLTISSLQAEDFATY
YCQQSYSIPQTFGQGTKLEIKGGGGSGGGGSGGGGSGGGGSGGGGSDIQM
TQTTSSLSASLGDRVTISCRASQDISKYLNWYQQKPDGTVKLLIYHTSRL
HSGVPSRFSGSGSGTDYSLTISNLEQEDIATYFCQQGNTLPYTFGGGTKL
EITGSTSGSGKPGSGEGSTKGEVKLQESGPGLVAPSQSLSVTCTVSGVSL
PDYGVSWIRQPPRKGLEWLGVIWGSETTYYNSALKSRLTIIKDNSKSQVF
LKMNSLQTDDTAIYYCAKHYYYGGSYAMDYWGQGTSVTVSSSGTTTPAPR
PPTPAPTIASQPLSLRPEACRPAAGGAVHTRGLDFACDIYIWAPLAGTCG
VLLLSLVITLYCKRGRKKLLYIFKQPFMRPVQTTQEEDGCSCRFPEEEEG
GCELRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTA
TKDTYDALHMQALPPR.

In an embodiment, the CAR construct (herein denoted TanCAR4) has the sequence:

(SEQ ID NO: 65)
MALPVTALLLPLALLLHAARPQVQLQQSGPGLVKPSQTLSLTCAISGDSV
SSNSAAWNWIRQSPSRGLEWLGRTYYRSKWYNDYAVSVKSRITINPDTSK
NQFSLQLNSVTPEDTAVYYCAREVTGDLEDAFDIWGQGTMVTVSSGGGGS
DIQMTQSPSSLSASVGDRVTITCRASQTIWSYLNWYQQRPGKAPNLLIYA
ASSLQSGVPSRFSGRGSGTDFTLTISSLQAEDFATYYCQQSYSIPQTFGQ
GTKLEIKGGGGSGGGGSGGGGSGGGGSDIQMTQTTSSLSASLGDRVTISC
RASQDISKYLNWYQQKPDGTVKLLIYHTSRLHSGVPSRFSGSGSGTDYSL
TISNLEQEDIATYFCQQGNTLPYTFGGGTKLEITGSTSGSGKPGSGEGST
KGEVKLQESGPGLVAPSQSLSVTCTVSGVSLPDYGVSWIRQPPRKGLEWL
GVIWGSETTYYNSALKSRLTIIKDNSKSQVFLKMNSLQTDDTAIYYCAKH
YYYGGSYAMDYWGQGTSVTVSSTSSGTTTPAPRPPTPAPTIASQPLSLRP
EACRPAAGGAVHTRGLDFACDIYIWAPLAGTCGVLLLSLVITLYCKRGRK
KLLYIFKQPFMRPVQTTQEEDGCSCRFPEEEEGGCELRVKFSRSADAPAY
QQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR.

In an embodiment, the CAR construct (herein denoted LoopCAR1) has the sequence:

(SEQ ID NO: 66)
ATMLLLVTSLLLCELPHPAFLLIPDIQMTQTTSSLSASLGDRVTISCRAS
QDISKYLNWYQQKPDGTVKLLIYHTSRLHSGVPSRFSGSGSGTDYSLTIS
NLEQEDIATYFCQQGNTLPYTFGGGTKLEITGGGGSGGGGSGGGGSQVQL
QQSGPGLVKPSQTLSLTCAISGDSVSSNSAAWNWIRQSPSRGLEWLGRTY
YRSKWYNDYAVSVKSRITINPDTSKNQFSLQLNSVTPEDTAVYYCAREVT
GDLEDAFDIWGQGTMVTVSSGGGGSDIQMTQSPSSLSASVGDRVTITCRA
SQTIWSYLNWYQQRPGKAPNLLIYAASSLQSGVPSRFSGRGSGTDFTLTI
SSLQAEDFATYYCQQSYSIPQTFGQGTKLEIKGGGGSGGGGSGGGGSEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSSGTTTPAPRPPTPAPTIASQPLSLRPEACRPAA
GGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR.

In an embodiment, the CAR construct (herein denoted LoopCAR2) has the sequence:

(SEQ ID NO: 67)
ATMLLLVTSLLLCELPHPAFLLIPDIQMTQTTSSLSASLGDRVTISCRAS
QDISKYLNWYQQKPDGTVKLLIYHTSRLHSGVPSRFSGSGSGTDYSLTIS
NLEQEDIATYFCQQGNTLPYTFGGGTKLEITGGCGSGGGGSGGGGSQVQL
QQSGPGLVKPSQTLSLTCAISGDSVSSNSAAWNWIRQSPSRGLEWLGRTY
YRSKWYNDYAVSVKSRITINPDTSKNQFSLQLNSVTPEDTAVYYCAREVT
GDLEDAFDIWGQGTMVTVSSGSTSGSGKPGSGEGSTKGDIQMTQSPSSLS
ASVGDRVTITCRASQTIWSYLNWYQQRPGKAPNLLIYAASSLQSGVPSRF
SGRGSGTDFTLTISSLQAEDFATYYCQQSYSIPQTFGQGTKLEIKGGGGS
GGGGSGGCGSEVKLQESGPGLVAPSQSLSVTCTVSGVSLPDYGVSWIRQP
PRKGLEWLGVIWGSETTYYNSALKSRLTIIKDNSKSQVFLKMNSLQTDDT
AIYYCAKHYYYGGSYAMDYWGQGTSVTVSSSGTTTPAPRPPTPAPTIASQ
PLSLRPEACRPAAGGAVHTRGLDFACDIYIWAPLAGTCGVLLLSLVITLY
CKRGRKKLLYIFKQPFMRPVQTTQEEDGCSCRFPEEEEGGCELRVKFSRS
ADAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHMQ
ALPPR.

In an embodiment, the CAR construct (herein denoted LoopCAR3) has the sequence:

(SEQ ID NO: 68)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSQVQLQQSGPGLV
KPSQTLSLTCAISGDSVSSNSAAWNWIRQSPSRGLEWLGRTYYRSKWYND
YAVSVKSRITINPDTSKNQFSLQLNSVTPEDTAVYYCAREVTGDLEDAFD
IWGQGTMVTVSSGSTSGSGKPGSGEGSTKGDIQMTQSPSSLSASVGDRVT
ITCRASQTIWSYLNWYQQRPGKAPNLLIYAASSLQSGVPSRFSGRGSGTD
FTLTISSLQAEDFATYYCQQSYSIPQTFGQGTKLEIKGGGGSGGGGSEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSTSSGTTTPAPRPPTPAPTIASQPLSLRPEACRP
AAGGAVHTRGLDFACDIYIWAPLAGTCGVLLLSLVITLYCKRGRKKLLYI
FKQPFMRPVQTTQEEDGCSCRFPEEEEGGCELRVKFSRSADAPAYKQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR.

In an embodiment, the CAR construct (herein denoted LoopCAR4) has the sequence:

(SEQ ID NO: 69)
MLLLVTSLLLCELPHPAFLLIPQVQLQQSGPGLVKPSQTLSLTCAISGDS
VSSNSAAWNWIRQSPSRGLEWLGRTYYRSKWYNDYAVSVKSRITINPDTS
KNQFSLQLNSVTPEDTAVYYCAREVTGDLEDAFDIWGQGTMVTVSSGGGG
SGGGGSDIQMTQTTSSLSASLGDRVTISCRASQDISKYLNWYQQKPDGTV
KLLIYHTSRLHSGVPSRFSGSGSGTDYSLTISNLEQEDIATYFCQQGNTL
PYTFGGGTKLEITGSTSGSGKPGSGEGSTKGEVKLQESGPGLVAPSQSLS
VTCTVSGVSLPDYGVSWIRQPPRKGLEWLGVIWGSETTYYNSALKSRLTI
IKDNSKSQVFLKMNSLQTDDTAIYYCAKHYYYGGSYAMDYWGQGTSVTVS
SGGGGSGGGGSDIQMTQSPSSLSASVGDRVTITCRASQTIWSYLNWYQQR
PGKAPNLLIYAASSLQSGVPSRFSGRGSGTDFTLTISSLQAEDFATYYCQ
QSYSIPQTFGQGTKLEIKTSSGTTTPAPRPPTPAPTIASQPLSLRPEACR
PAAGGAVHTRGLDFACDIYIWAPLAGTCGVLLLSLVITLYCKRGRKKLLY
IFKQPFMRPVQTTQEEDGSCRFPEEEEGGCELRVKFSRSADAPAYKQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR.

In an embodiment, the CAR construct (herein denoted LoopCAR5) has the sequence:

(SEQ ID NO: 70)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGGGGSGGGGSCGGGSQVQLQQ
SGPGLVKPSQTLSLTCAISGDSVSSNSAAWNWIRQSPSRGLEWLGRTYYR
SKWYNDYAVSVKSRITINPDTSKNQFSLQLNSVTPEDTAVYYCAREVTGD
LEDAFDIWGQGTMVTVSSGGGGSGGGGSDIQMTQSPSSLSASVGDRVTIT
CRASQTIWSYLNWYQQRPGKAPNLLIYAASSLQSGVPSRFSGRGSGTDFT
LTISSLQAEDFATYYCQQSYSIPQTFGQGTKLEIKGGGGCGGGGSGGGGS
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSTSSGTTTPAPRPPTPAPTIASQPLSLRPEA
CRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYKQ
GQNQLYNELNLGRREEYDVLDKRRGRDPEMGGKPRRKNPQEGLYNELQKD
KMAEAYSEIGMKGERRRGKGHDGLYQGLSTATKDTYDALHMQALPPR.

The inventive CAR constructs may provide many advantages. In an embodiment of the invention, for example, the inventive CAR constructs may, advantageously, reduce or prevent cancer cell escape due to loss of expression of one antigen, e.g., CD19 or CD22, by the cancer cell. For example, it is believed that the inventive CAR constructs may reduce or prevent relapses that have been observed in cancer patients following treatment with a CAR having antigenic specificity for only CD19 or CD22 and whose cancer has lost expression of that antigen. Also, the inventive CAR constructs may also be advantageous for treating patients who have heterogeneous level of expression of CD19 or CD22. The inventive CAR constructs may also increase the patient population that may be successfully treated. For example, a patient that may fail to respond to a CAR therapy that targets only CD19 may respond to a CAR therapy that targets CD22, and a patient that may fail to respond to a CAR therapy that targets only CD22 may respond to a CAR therapy that targets CD19. Additionally, regarding the inventive cleavable CARs, co-transduction of T cells using two vectors, each having a single CAR, provides only a low percentage of cells expressing both CARs and substantial numbers of T cells expressing one or the other CAR only; an advantage of using the inventive cleavable CAR constructs is that there may be equal or substantially equal expression of each CAR in each T cell that successfully integrates the construct. Moreover, by targeting both CD19 and CD22, the inventive cleavable and non-cleavable CAR constructs may, advantageously, provide synergistic responses as compared to therapies which target only a single antigen, and may also provide a more broadly active therapy to patients with heterogeneous expression of one or both of CD19 and CD22 on cancer cells.

Thus, without being bound to a particular theory or mechanism, it is believed that by eliciting an antigen-specific response against two antigens, e.g., CD22 and CD19, the inventive CAR constructs provide for one or more of any of the following: targeting and destroying CD22-expressing cancer cells, targeting and destroying CD19-expressing cancer cells, reducing or eliminating cancer cells, facilitating infiltration of immune cells to tumor site(s), and enhancing/extending anti-cancer responses.

Included in the scope of the invention are functional portions of the inventive CAR constructs described herein. The term “functional portion” when used in reference to a CAR refers to any part or fragment of the CAR constructs of the invention, which part or fragment retains the biological activity of the CAR construct of which it is a part (the parent CAR construct). Functional portions encompass, for example, those parts of a CAR construct that retain the ability to recognize target cells, or detect, treat, or prevent cancer, to a similar extent, the same extent, or to a higher extent, as the parent CAR construct. In reference to the parent CAR construct, the functional portion can comprise, for instance, about 10%, about 25%, about 30%, about 50%, about 68%, about 80%, about 90%, about 95%, or more, of the parent CAR.

The functional portion can comprise additional amino acids at the amino or carboxy terminus of the portion, or at both termini, which additional amino acids are not found in the amino acid sequence of the parent CAR construct. Desirably, the additional amino acids do not interfere with the biological function of the functional portion, e.g., recognize target cells, detect cancer, treat or prevent cancer, etc. More desirably, the additional amino acids enhance the biological activity as compared to the biological activity of the parent CAR construct.

Included in the scope of the invention are functional variants of the inventive CAR constructs described herein. The term “functional variant,” as used herein, refers to a CAR construct, polypeptide, or protein having substantial or significant sequence identity or similarity to a parent CAR construct, which functional variant retains the biological activity of the CAR of which it is a variant. Functional variants encompass, for example, those variants of the CAR construct described herein (the parent CAR construct) that retain the ability to recognize target cells to a similar extent, the same extent, or to a higher extent, as the parent CAR construct. In reference to the parent CAR construct, the functional variant can, for instance, be at least about 30%, about 50%, about 75%, about 80%, about 90%, about 91%, about 92%, about 93%, about 94%, about 95%, about 96%, about 97%, about 98%, about 99% or more identical in amino acid sequence to the parent CAR construct.

A functional variant can, for example, comprise the amino acid sequence of the parent CAR with at least one conservative amino acid substitution. Alternatively or additionally, the functional variants can comprise the amino acid sequence of the parent CAR construct with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR construct.

Amino acid substitutions of the inventive CAR constructs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The CAR construct can consist essentially of the specified amino acid sequence or sequences described herein, such that other components, e.g., other amino acids, do not materially change the biological activity of the functional variant.

The CAR constructs of embodiments of the invention (including functional portions and functional variants) can be of any length, i.e., can comprise any number of amino acids, provided that the CAR constructs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CAR constructs of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, cx-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The CAR constructs of embodiments of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CAR constructs of embodiments of the invention (including functional portions and functional variants thereof) can be obtained by methods known in the art. The CAR constructs may be made by any suitable method of making polypeptides or proteins, including de novo synthesis. Also, the CAR constructs can be recombinantly produced using the nucleic acids described herein using standard recombinant methods. See, for instance, Green et al., Molecular Cloning: A Laboratory Manual, 4th ed., Cold Spring Harbor Press, Cold Spring Harbor, NY 2012. Further, portions of some of the CAR constructs of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CAR constructs described herein (including functional portions and functional variants thereof) can be commercially synthesized by companies, such as Synpep (Dublin, CA), Peptide Technologies Corp. (Gaithersburg, MD), and Multiple Peptide Systems (San Diego, CA). In this respect, the inventive CAR constructs can be synthetic, recombinant, isolated, and/or purified.

Another embodiment of the invention provides chimeric antigen receptor (CAR) amino acid construct comprising (a) two or more cleavable domains; (b) a first CAR comprising a first antigen binding domain, a first transmembrane domain, and a first intracellular T cell signaling domain; and (c) a second CAR comprising a second antigen binding domain, a second transmembrane domain, and a second intracellular T cell signaling domain; wherein the first and second CARs are linked through the two or more cleavable domains. In an embodiment, the two or more cleavable domains are immediately adjacent or have at least one linker between at least two cleavable domains. In an embodiment, there are exactly two cleavable domains.

Another embodiment of the invention provides a method of making a chimeric antigen receptor (CAR) amino acid construct, the method comprising designing two or more cleavable domains between (a) a first CAR comprising a first antigen binding domain, a first transmembrane domain, and a first intracellular T cell signaling domain; and (b) a second CAR comprising a second antigen binding domain, a second transmembrane domain, and a second intracellular T cell signaling domain; wherein the first and second CARs are linked through the two or more cleavable domains; and cloning into a plasmid a sequence comprising from N-terminus to C-terminus the first CAR, the two or more cleavable domains, and the second CAR. In an embodiment, the two or more cleavable domains are immediately adjacent or have at least one linker between at least two cleavable domains. In an embodiment, there are exactly two cleavable domains.

Further provided by an embodiment of the invention is a nucleic acid comprising a nucleotide sequence encoding any of the CAR constructs described herein (including functional portions and functional variants thereof). The nucleic acids of the invention may comprise a nucleotide sequence encoding any of the leader sequences, antigen binding domains, transmembrane domains, linkers, and/or intracellular T cell signaling domains described herein.

In an embodiment, the nucleic acid comprises a nucleotide sequence that encodes any CAR construct described herein. In an embodiment of the invention, the nucleic acid may comprise, consist of, or consist essentially of, the nucleotide sequence of SEQ ID NO: 53 (an anti-CD19/anti-CD22 V1 CAR), SEQ ID NO: 54 (an anti-CD19/anti-CD22 V5 CAR), SEQ ID NO: 55 (an anti-CD19/anti-CD22 V6 CAR), SEQ ID NO: 56 (an anti-CD19/anti-CD22 V7 CAR), SEQ ID NO: 57 (an anti-CD19/anti-CD22 V8 CAR), SEQ ID NO: 71 (a TanCAR2), SEQ ID NO: 72 (a TanCAR3), SEQ ID NO: 73 (a TanCAR4), SEQ ID NO: 74 (a LoopCAR1), SEQ ID NO: 75 (a LoopCAR2), SEQ ID NO: 76 (a LoopCAR3), SEQ ID NO: 77 (a LoopCAR4), or SEQ ID NO: 78 (a LoopCAR5).

“Nucleic acid,” as used herein, includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions. In some embodiments, the nucleic acid may encode additional amino acid sequences that do not affect the function of the CAR construct and which may or may not be translated upon expression of the nucleic acid by a host cell.

The nucleic acids of an embodiment of the invention may be recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Green et al., supra. The nucleic acids can be constructed based on chemical synthesis and/or enzymatic ligation reactions using procedures known in the art. See, for example, Green et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, CO) and Synthegen (Houston, TX).

The nucleic acid can comprise any isolated or purified nucleotide sequence which encodes any of the CAR constructs or functional portions or functional variants thereof. Alternatively, the nucleotide sequence can comprise a nucleotide sequence which is degenerate to any of the sequences or a combination of degenerate sequences.

An embodiment of the invention also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive CAR constructs. It is generally appreciated that conditions can be rendered more stringent by the addition of increasing amounts of formamide.

The invention also provides a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In an embodiment, the nucleic acids of the invention can be incorporated into a recombinant expression vector. In this regard, an embodiment of the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector. An exemplary vector backbone is the lenti-vector backbone of SEQ ID NO: 58.

In an embodiment, the recombinant expression vector of the invention can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, MD), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA). Bacteriophage vectors, such as λGT10, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-Cl, pMAM, and pMAMneo (Clontech). The recombinant expression vector may be a viral vector, e.g., a retroviral vector or a lentiviral vector.

In an embodiment, the recombinant expression vectors of the invention can be prepared using standard recombinant DNA techniques described in, for example, Green et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may also comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR construct (including functional portions and functional variants thereof), or to the nucleotide sequence which is complementary to or which hybridizes to the nucleotide sequence encoding the CAR construct. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine daminase, purine nucleoside phosphorylase, and nitroreductase.

Included in the scope of the invention are conjugates, e.g., bioconjugates, comprising any of the inventive CAR constructs (including any of the functional portions or variants thereof), nucleic acids, recombinant expression vectors, host cells, or populations of host cells. Conjugates, as well as methods of synthesizing conjugates in general, are known in the art.

An embodiment of the invention further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5α E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5α cell. For purposes of producing a recombinant CAR construct, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a T cell or natural killer cell (NK cell).

For purposes herein, the T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4⁺/CD8⁺ double positive T cells, CD4⁺ helper T cells, e.g., Th₁ and Th₂ cells, CD8⁺ T cells (e.g., cytotoxic T cells), tumor infiltrating cells, memory T cells, naïve T cells, and the like. The T cell may be a CD8⁺ T cell or a CD4⁺ T cell.

Also provided by an embodiment of the invention is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

The inventive CAR constructs (including functional portions and variants thereof), nucleic acids, recombinant expression vectors, and host cells (including populations thereof), all of which are collectively referred to as “inventive CAR construct materials” hereinafter, can be isolated and/or purified. The term “isolated,” as used herein, means having been removed from its natural environment. The term “purified” or “isolated” does not require absolute purity or isolation; rather, it is intended as a relative term. Thus, for example, a purified (or isolated) host cell preparation is one in which the host cell is more pure than cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

The inventive CAR construct materials can be formulated into a composition, such as a pharmaceutical composition. In this regard, an embodiment of the invention provides a pharmaceutical composition comprising any of the inventive CAR construct materials described herein and a pharmaceutically acceptable carrier. The inventive pharmaceutical compositions containing any of the inventive CAR construct materials can comprise more than one inventive CAR construct material, e.g., a CAR construct and a nucleic acid, or two or more different CAR constructs. Alternatively, the pharmaceutical composition can comprise an inventive CAR construct material in combination with other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In a preferred embodiment, the pharmaceutical composition comprises the inventive host cell or populations thereof.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which has no detrimental side effects or toxicity under the conditions of use.

The choice of carrier will be determined in part by the particular inventive CAR construct material, as well as by the particular method used to administer the inventive CAR construct material. Accordingly, there are a variety of suitable formulations of the pharmaceutical composition of the invention. Methods for preparing administrable (e.g., parenterally administrable) compositions are known or apparent to those skilled in the art and are described in more detail in, for example, Remington: The Science and Practice of Pharmacy, Pharmaceutical Press; 22nd ed. (2012).

The inventive CAR construct materials may be administered in any suitable manner. Preferably, the inventive CAR construct materials are administered by injection, (e.g., subcutaneously, intravenously, intratumorally, intraarterially, intramuscularly, intradermally, interperitoneally, or intrathecally). Preferably, the inventive CAR construct materials are administered intravenously. A suitable pharmaceutically acceptable carrier for the inventive CAR construct material for injection may include any isotonic carrier such as, for example, normal saline (about 0.90% w/v of NaCl in water, about 300 mOsm/L NaCl in water, or about 9.0 g NaCl per liter of water), NORMOSOL R electrolyte solution (Abbott, Chicago, IL), PLASMA-LYTE A (Baxter, Deerfield, IL), about 5% dextrose in water, or Ringer's lactate. In an embodiment, the pharmaceutically acceptable carrier is supplemented with human serum albumen.

An “effective amount” or “an amount effective to treat” refers to a dose that is adequate to prevent or treat cancer in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the inventive CAR construct materials in each or various rounds of administration. By way of example and not intending to limit the invention, when the inventive CAR construct material is a host cell, an exemplary dose of host cells may be a minimum of one million cells (1×10⁶ cells/dose).

For purposes of the invention, the amount or dose of the inventive CAR construct material administered should be sufficient to effect a therapeutic or prophylactic response in the subject or animal over a reasonable time frame. For example, the dose of the inventive CAR construct material should be sufficient to bind to antigen, or detect, treat or prevent cancer in a period of from about 2 hours or longer, e.g., about 12 to about 24 or more hours, from the time of administration. In certain embodiments, the time period could be even longer. The dose will be determined by the efficacy of the particular inventive CAR construct material and the condition of the animal (e.g., human), as well as the body weight of the animal (e.g., human) to be treated.

For purposes of the invention, an assay, which comprises, for example, comparing the extent to which target cells are lysed and/or IFN-γ is secreted by T cells expressing the released CARs of the inventive CAR construct upon administration of a given dose of such T cells to a mammal, among a set of mammals of which is each given a different dose of the T cells, could be used to determine a starting dose to be administered to a mammal. The extent to which target cells are lysed and/or IFN-γ is secreted upon administration of a certain dose can be assayed by methods known in the art.

When the inventive CAR construct materials are administered with one or more additional therapeutic agents, one or more additional therapeutic agents can be coadministered to the mammal. By “coadministering” is meant administering one or more additional therapeutic agents and the inventive CAR construct materials sufficiently close in time such that the inventive CAR construct materials can enhance the effect of one or more additional therapeutic agents, or vice versa. In this regard, the inventive CAR construct materials can be administered first and the one or more additional therapeutic agents can be administered second, or vice versa. Alternatively, the inventive CAR construct materials and the one or more additional therapeutic agents can be administered simultaneously. An exemplary therapeutic agent that can be co-administered with the CAR construct materials is IL-2. It is believed that IL-2 enhances the therapeutic effect of the inventive CAR construct materials.

It is contemplated that the inventive CAR construct materials can be used in methods of treating or preventing a disease in a mammal. Without being bound to a particular theory or mechanism, the inventive CAR constructs have biological activity, e.g., ability to release/cleave CARs that recognize antigen, e.g., one or both of CD19 and CD22, such that the released CARS, when expressed by a cell, are able to mediate an immune response against the cell expressing the antigen, e.g., one or both of CD19 and CD22. In this regard, an embodiment of the invention provides a method of treating or preventing cancer in a mammal, comprising administering to the mammal any of the CAR constructs, the nucleic acids, the recombinant expression vectors, the host cells, the population of cells, and/or the pharmaceutical compositions of the invention in an amount effective to treat or prevent cancer in the mammal.

An embodiment of the invention further comprises lymphodepleting the mammal prior to administering the inventive CAR construct materials. Examples of lymphodepletion include, but may not be limited to, nonmyeloablative lymphodepleting chemotherapy, myeloablative lymphodepleting chemotherapy, total body irradiation, etc.

For purposes of the inventive methods, wherein host cells or populations of cells are administered, the cells can be cells that are allogeneic or autologous to the mammal. Preferably, the cells are autologous to the mammal.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the inventive methods of treatment, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., medulloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia (CLL), chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma), lymphoma, malignant mesothelioma, mastocytoma, melanoma, multiple myeloma, nasopharynx cancer, non-Hodgkin lymphoma, B-chronic lymphocytic leukemia, B-precursor acute lymphoblastic leukemia (B-ALL), pre-B cell precursor acute lymphoblastic leukemia (BCP-ALL), B cell lymphoma, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, and ureter cancer. Preferably, the cancer is a hematological malignancy (e.g., leukemia or lymphoma, including but not limited to Hodgkin lymphoma, non-Hodgkin lymphoma, CLL, acute lymphocytic cancer, acute myeloid leukemia, B-chronic lymphocytic leukemia, hairy cell leukemia, acute lymphocytic leukemia (ALL) (also referred to as “acute lymphoblastic leukemia”), B-ALL, BCP-ALL, B cell lymphoma, and Burkitt's lymphoma). Preferably, the cancer is characterized by the expression of one or both of CD22 and CD19, and more preferably is a hematological malignancy that is characterized by the expression of one or both of CD19 and CD22.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of cancer in a mammal. Furthermore, the treatment or prevention provided by the inventive method can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment of the invention provides a use of the inventive CAR constructs, nucleic acids, recombinant expression vectors, host cells, populations of cells, or pharmaceutical compositions, for the treatment or prevention of cancer in a mammal.

Another embodiment of the invention provides a method of detecting the presence of cancer in a mammal, comprising: (a) contacting a sample comprising one or more cells from the mammal with the CAR constructs, the nucleic acids, the recombinant expression vectors, the host cells, the population of cells, or the pharmaceutical compositions of the invention, thereby forming a complex, (b) and detecting the complex, wherein detection of the complex is indicative of the presence of cancer in the mammal.

The sample may be obtained by any suitable method, e.g., biopsy or necropsy. A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer.

With respect to an embodiment of the inventive method of detecting the presence of cancer in a mammal, the sample comprising cells of the mammal can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the mammal, e.g., the cells of any organ or tissue, including blood cells or endothelial cells.

For purposes of the inventive detecting method, the contacting can take place in vitro or in vivo with respect to the mammal. Preferably, the contacting is in vitro.

Also, detection of the complex can occur through any number of ways known in the art. For instance, the inventive CAR constructs, nucleic acids, recombinant expression vectors, host cells, or populations of cells, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

Methods of testing a CAR for the ability to recognize target cells and for antigen specificity are known in the art. For instance, Clay et al., J. Immunol., 163:507-513 (1999), teaches methods of measuring the release of cytokines (e.g., interferon-γ, granulocyte/monocyte colony stimulating factor (GM-CSF), tumor necrosis factor α (TNF-α) or interleukin 2 (IL-2)). In addition, CAR function can be evaluated by measurement of cellular cytoxicity, as described in Zhao et al., J. Immunol., 174:4415-4423 (2005).

The following includes certain aspects of the invention.

1. A chimeric antigen receptor (CAR) amino acid construct comprising:
(a) a cleavable domain;
(b) a first CAR comprising
   a first antigen binding domain,
   a first transmembrane domain, and
   a first intracellular T cell signaling domain; and
(c) a second CAR comprising
   a second antigen binding domain,
   a second transmembrane domain, and
   a second intracellular T cell signaling domain;
wherein the first and second CARs are linked through the cleavable domain,
wherein the first antigen binding domain comprises an antigen binding domain of the m971 antibody,
wherein when the first CAR is cleaved from the construct, the first antigen binding domain has antigenic specificity for CD22.

2. The CAR construct according to aspect 1, wherein cleaving the cleavable domain releases the first and second CARs from the CAR construct.

3. The CAR construct according to aspect 1 or 2, wherein the first antigen binding domain comprises a heavy chain variable region comprising the amino acid sequences of SEQ ID NOs: 3-9 and a light chain variable region comprising the amino acid sequences of SEQ ID NOs: 11-17.

4. The CAR construct according to any one of aspects 1-3, wherein the first antigen binding domain comprises the amino acid sequences of SEQ ID NOs: 3-9 and 11-17.

5. The CAR construct according to any one of aspects 1-4, wherein, when the second CAR is cleaved from the construct, the second antigen binding domain has antigenic specificity for CD19.

6. The CAR construct according to any one of aspects 1-5, wherein the second antigen binding domain comprises an antigen binding domain of the FMC63 antibody.

7. The CAR construct according to any one of aspects 1-6, wherein the second antigen binding domain comprises a heavy chain variable region comprising the amino acid sequences of SEQ ID NOs: 31-37 and a light chain variable region comprising the amino acid sequences of SEQ ID NOs: 23-29.

8. The CAR construct according to any one of aspects 1-7, wherein the second antigen binding domain comprises the amino acid sequences of SEQ ID NO: 23-29 and 31-37.

9. A chimeric antigen receptor (CAR) amino acid construct comprising:
(a) a cleavable domain;
(b) a first CAR comprising
   a first antigen binding domain,
   a first transmembrane domain, and
   a first intracellular T cell signaling domain; and
(c) a second CAR comprising
   a second antigen binding domain,
   a second transmembrane domain, and
   a second intracellular T cell signaling domain;
wherein the first and second CARs are linked through the cleavable domain,
wherein the first antigen binding domain comprises an antigen binding domain of the FMC63 antibody,
wherein when the first CAR is cleaved from the construct, the first antigen binding domain has antigenic specificity for CD19.

10. The CAR construct according to aspect 9, wherein cleaving the cleavable domain releases the first and second CARs from the CAR construct.

11. The CAR construct according to aspect 9 or 10, wherein the first antigen binding domain comprises a heavy chain variable region comprising the amino acid sequences of SEQ ID NO: 31-37 and a light chain variable region comprising the amino acid sequences of SEQ ID NOs: 23-29.

12. The CAR construct according to any one of aspects 9-11, wherein the first antigen binding domain comprises the amino acid sequences of SEQ ID NOs: 23-29 and 31-37.

13. The CAR construct according to any one of aspects 9-12, wherein, when the second CAR is cleaved from the construct, the second antigen binding domain has antigenic specificity for CD22.

14. The CAR construct according to any one of aspects 1-13, wherein the first or second transmembrane domain comprises a CD8 transmembrane domain and a CD8 hinge domain.

15. The CAR construct according to aspect 14, wherein the CD8 transmembrane domain comprises the amino acid sequence of SEQ ID NO: 19 and the CD8 hinge domain comprises the amino acid sequence of SEQ ID NO: 18.

16. The CAR construct according to any one of aspects 1-15, wherein the first or second intracellular T cell signaling domain comprises a 4-1BB intracellular T cell signaling sequence.

17. The CAR construct according to aspect 16, wherein the 4-1BB intracellular T cell signaling sequence comprises the amino acid sequence of SEQ ID NO: 20.

18. The CAR construct according to any one of aspects 1-17, wherein the first or second intracellular T cell signaling domain comprises a CD3 zeta (ζ) intracellular T cell signaling sequence.

19. The CAR construct according to aspect 18, wherein the CD3ζ intracellular T cell signaling sequence comprises the amino acid sequence of SEQ ID NO: 21.

20. The CAR construct according to any one of aspects 1-19, wherein the cleavable domain is 2A or furin.

21. The CAR construct according to any one of aspects 1-20, wherein the CAR construct comprises exactly two CARs being the first and second CARs, respectively.

22. A chimeric antigen receptor (CAR) amino acid construct comprising the amino acid sequence of SEQ ID NO: 48, 49, 50, 51, or 52.

23. A chimeric antigen receptor (CAR) amino acid construct comprising an amino acid sequence having 90%, 91%, 92%, 93%, 94%, 95%, 96%, 97%, 98%, 99% or greater sequence identity (e.g., 100%) with any one of SEQ ID NOS: 63-70.

24. A chimeric antigen receptor (CAR) amino acid construct comprising:
(a) two or more cleavable domains;
(b) a first CAR comprising
   a first antigen binding domain,
   a first transmembrane domain, and
   a first intracellular T cell signaling domain; and
(c) a second CAR comprising
   a second antigen binding domain,
   a second transmembrane domain, and
   a second intracellular T cell signaling domain;
wherein the first and second CARs are linked through the two or more cleavable domains.

25. The CAR construct of aspect 24, wherein the two or more cleavable domains are immediately adjacent or have at least one linker between at least two cleavable domains.

26. The CAR construct of aspect 25 or 24, wherein there are exactly two cleavable domains.

27. A nucleic acid comprising a nucleotide sequence encoding the CAR construct of any one of aspects 1-26.

28. The nucleic acid according to aspect 27, comprising the nucleotide sequence of any one of SEQ ID NOs: 53-57 or 71-78.

29. A recombinant expression vector comprising the nucleic acid of aspect 27 or 28.

30. An isolated host cell comprising the recombinant expression vector of aspect 29.

31. A population of cells comprising at least one host cell of aspect 30.

32. A pharmaceutical composition comprising the CAR construct of any one of aspects 1-26, the nucleic acid of aspect 27 or 28, the recombinant expression vector of aspect 29, the host cell of aspect 30, or the population of cells of aspect 31, and a pharmaceutically acceptable carrier.

33. A method of detecting the presence of cancer in a mammal, comprising:
(a) contacting a sample comprising one or more cells from the mammal with the CAR construct of any one of aspects 1-26, the nucleic acid of aspect 27 or 28, the recombinant expression vector of aspect 29, the host cell of aspect 30, the population of cells of aspect 31, or the pharmaceutical composition of aspect 32, thereby forming a complex, and
(b) detecting the complex, wherein detection of the complex is indicative of the presence of cancer in the mammal.

34. The CAR construct of any one of aspects 1-26, the nucleic acid of aspect 27 or 28, the recombinant expression vector of aspect 29, the host cell of aspect 30, the population of cells of aspect 31, or the pharmaceutical composition of aspect 32 for use in the treatment or prevention of cancer in a mammal.

35. The CAR construct for the use of aspect 34, wherein the cancer is a hematological malignancy.

36. A method of making a chimeric antigen receptor (CAR) amino acid construct, the method comprising designing two or more cleavable domains between
(a) a first CAR comprising
   a first antigen binding domain,
   a first transmembrane domain, and
   a first intracellular T cell signaling domain; and
(b) a second CAR comprising
   a second antigen binding domain,
   a second transmembrane domain, and
   a second intracellular T cell signaling domain;
wherein the first and second CARs are linked through the two or more cleavable domains;
and
cloning into a plasmid a sequence comprising from N-terminus to C-terminus the first CAR, the two or more cleavable domains, and the second CAR.

37. The method of aspect 36, wherein the two or more cleavable domains are immediately adjacent or have at least one linker between at least two cleavable domains.

38. The method of aspect 36 or 37, wherein there are exactly two cleavable domains.

The following examples further illustrate the invention but, of course, should not be construed as in any way limiting its scope.

### Example 1

This example demonstrates generation of CAR constructs, CAR construct-encoding lentiviral vectors, and CAR-expressing T cells, in accordance with embodiments of the invention, as well as generation of other CARs for comparison.

The CAR constructs were synthesized by GENEWIZ (South Plainfield, NJ, USA) and then sub-cloned into the lenti-viral plasmid backbone between NhE1 and HincII sites.

CAR construct-encoding lentiviral vectors were produced by transient transfection of the 293T cell line. Briefly, 293T cells were plated into poly-D lysine coated 15 cm plates (BD Biosciences, San Jose, CA, USA). The following day, 293T cells were transfected using lipofectamine 3000 (Life Technologies, Calrsbad, CA, USA) with plasmids encoding the CAR construct along with packaging and envelope vectors (pMDLg/pRRE, pMD-2G, and pRSV-Rev). Lentiviral supernatants were collected 48-72 hours post-transfection, centrifuged at 3000 RPM for 10 minutes to remove cell debris, then stored at −80° C. Human PBMCs from normal donors were activated with a 1:1 ratio of CD3/CD28 microbeads (Life Technologies) in AIM-V media containing 40 IU/mL recombinant IL-2 (rhIL-2; Roche, Basel, Switzerland) for 24 hours. Activated T cells were resuspended at 2 million cells per 3 mL of lentiviral supernatant plus 1 mL of fresh AIM-V media with 10 μg/mL protamine sulfate and 100 IU/mL IL-2 and cultured in 6-well plates. Plates were centrifuged at 1000×g for 2 hours at 32° C. and then incubated at 37° C. overnight. A second transduction was performed the following day. On the third day, the CD3/CD28 beads were removed, and the cells were cultured at 300,000 cells/mL in AIM-V containing 100 IU/mL IL-2 with fresh IL2-containing media added every 2-3 days until harvest at day 8 or 9.

Vectors for the single anti-CD19 CAR, the single anti-CD22 CAR, and the bispecific LoopCAR6 were produced by transient transfection of the 293T lenti packaging cell line.

The sequence of the anti-CD19 CAR is below:

(SEQ ID NO: 59)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGSTSGSGKPGSGEGSTKGEVK
LQESGPGLVAPSQSLSVTCTVSGVSLPDYGVSWIRQPPRKGLEWLGVIWG
SETTYYNSALKSRLTIIKDNSKSQVFLKMNSLQTDDTAIYYCAKHYYYGG
SYAMDYWGQGTSVTVSSSGTTTPAPRPPTPAPTIASQPLSLRPEACRPAA
GGAVHTRGLDFACDIYIWAPLAGTCGVLLLSLVITLYCKRGRKKLLYIFK
QPFMRPVQTTQEEDGCSCRFPEEEEGGCELRVKFSRSADAPAYQQGQNQL
YNELNLGRREEYDVLDKRRGRDPEMGGKPRRKNPQEGLYNELQKDKMAEA
YSEIGMKGERRRGKGHDGLYQGLSTATKDTYDALHMQALPPR.

The sequence of the anti-CD22 CAR is below:

(SEQ ID NO: 60)
MLLLVTSLLLCELPHPAFLLIPQVQLQQSGPGLVKPSQTLSLTCAISGDS
VSSNSAAWNWIRQSPSRGLEWLGRTYYRSKWYNDYAVSVKSRITINPDTS
KNQFSLQLNSVTPEDTAVYYCAREVTGDLEDAFDIWGQGTMVTVSSGGGG
SDIQMTQSPSSLSASVGDRVTITCRASQTIWSYLNWYQQRPGKAPNLLIY
AASSLQSGVPSRFSGRGSGTDFTLTISSLQAEDFATYYCQQSYSIPQTFG
QGTKLEIKTSSGTTTPAPRPPTPAPTIASQPLSLRPEACRPAAGGAVHTR
GLDFACDIYIWAPLAGTCGVLLLSLVITLYCKRGRKKLLYIFKQPFMRPV
QTTQEEDGCSCRFPEEEEGGCELRVKFSRSADAPAYQQGQNQLYNELNLG
RREEYDVLDKRRGRDPEMGGKPRRKNPQEGLYNELQKDKMAEAYSEIGMK
GERRRGKGHDGLYQGLSTATKDTYDALHMQALPPR.

The LoopCAR6 is described in International Patent Publication No. WO 2016/149578 and has the following sequence:

(SEQ ID NO: 61)
MLLLVTSLLLCELPHPAFLLIPDIQMTQTTSSLSASLGDRVTISCRASQD
ISKYLNWYQQKPDGTVKLLIYHTSRLHSGVPSRFSGSGSGTDYSLTISNL
EQEDIATYFCQQGNTLPYTFGGGTKLEITGGGGSQVQLQQSGPGLVKPSQ
TLSLTCAISGDSVSSNSAAWNWIRQSPSRGLEWLGRTYYRSKWYNDYAVS
VKSRITINPDTSKNQFSLQLNSVTPEDTAVYYCAREVTGDLEDAFDIWGQ
GTMVTVSSGSTSGSGKPGSGEGSTKGDIQMTQSPSSLSASVGDRVTITCR
ASQTIWSYLNWYQQRPGKAPNLLIYAASSLQSGVPSRFSGRGSGTDFTLT
ISSLQAEDFATYYCQQSYSIPQTFGQGTKLEIKGGGGSEVKLQESGPGLV
APSQSLSVTCTVSGVSLPDYGVSWIRQPPRKGLEWLGVIWGSETTYYNSA
LKSRLTIIKDNSKSQVFLKMNSLQTDDTAIYYCAKHYYYGGSYAMDYWGQ
GTSVTVSSSGTTTPAPRPPTPAPTIASQPLSLRPEACRPAAGGAVHTRGL
DFACDIYIWAPLAGTCGVLLLSLVITLYCKRGRKKLLYIFKQPFMRPVQT
TQEEDGCSCRFPEEEEGGCELRVKFSRSADAPAYKQGQNQLYNELNLGRR
EEYDVLDKRRGRDPEMGGKPRRKNPQEGLYNELQKDKMAEAYSEIGMKGE
RRRGKGHDGLYQGLSTATKDTYDALHMQALPPR.

### Example 2

This example demonstrates surface expression on human T cells of CARs, cleaved from a CAR construct, in accordance with embodiments of the invention, compared to other CARs.

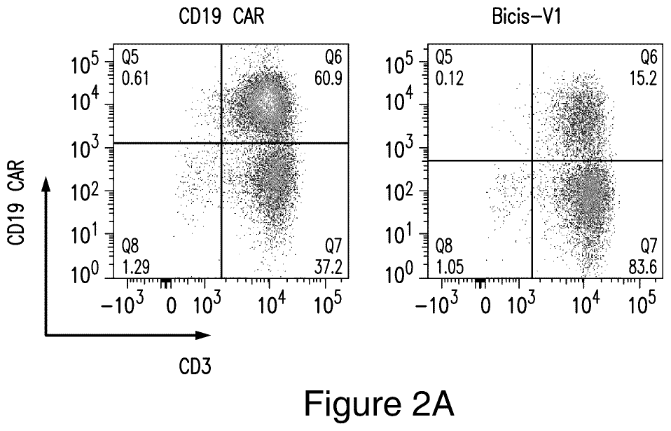

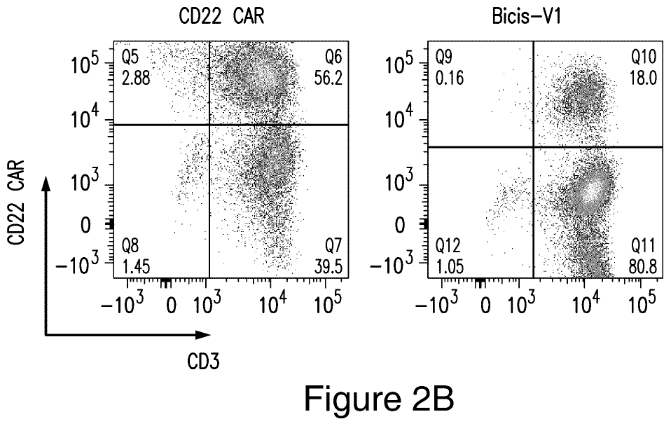

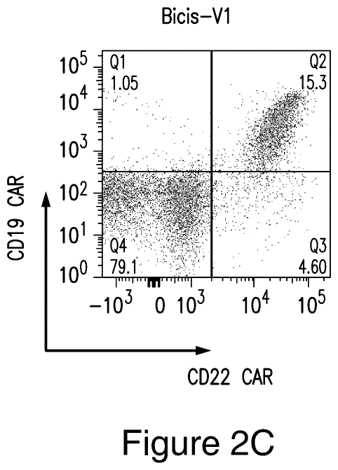

Surface expression of anti-CD19 CAR and anti-CD22 CAR on the V1 transduced T cells is about 15%, while the expression of the anti-CD19 CAR from a vector encoding only the single anti-CD19 CAR is 61% and expression of the anti-CD22 CAR from a vector encoding only the single anti-CD22 CAR is 56% (FIGS. 2A-2C).

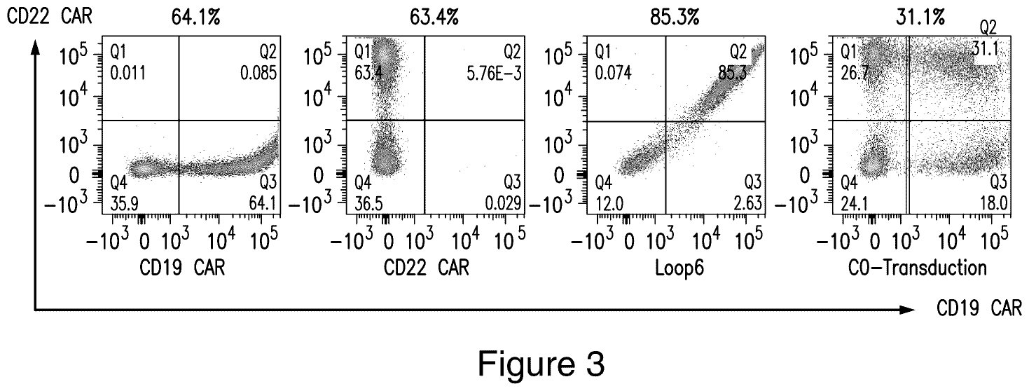

Human PBMCs from a healthy donor were activated with CD3/CD28 microbeads for 24 hours. Activated T cells were then transduced with vector individually or co-transduced with both the single anti-CD19 CAR and the single anti-CD22 CAR vectors together. Surface expression of the anti-CD19 CAR and the anti-CD22 CAR were analyzed on day 8. Cotransduced T cells had much lower expression of both anti-CD19 and anti-CD22 CARS compared to the bispecific LoopCAR6. The expression of anti-CD19 and anti-CD22 CARs on co-transduced T cells is not at an equal molar ratio. In contrast, the LoopCAR6 showed an almost 1:1 ratio in the expression of anti-CD19 and anti-CD22 CARs, which displays as a diagonal plot. See FIG. 3.

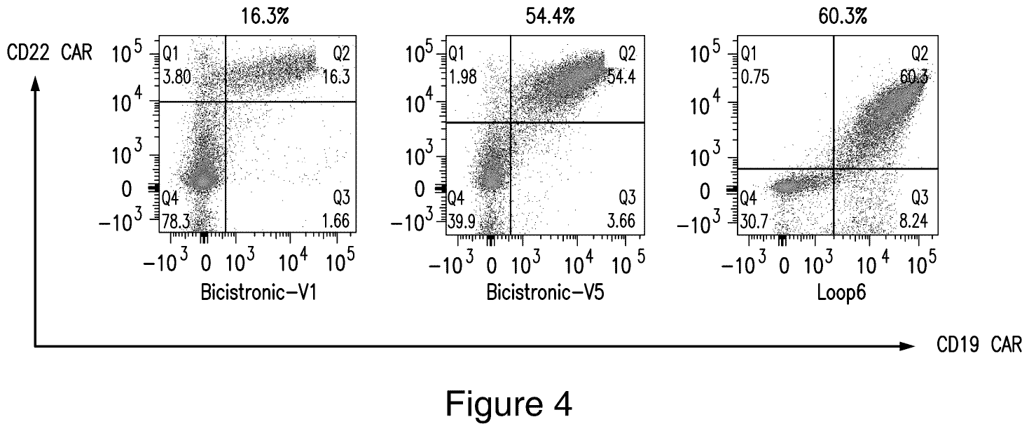

Vectors for the bispecific LoopCAR6 and V1 and V5 CARs were produced by transient transfection of the 293T lenti packaging cell line. Human PBMCs from a healthy donor were activated with CD3/CD28 microbeads for 24 hours. Activated T cells were then transduced with the vectors. Surface expression of anti-CD19 CAR and anti-CD22 CAR were analyzed on day 7 using flow cytometry. T cells transduced with a vector encoding the V5 CAR have higher cell surface expression of both the separated anti-CD19 CAR and the separated anti-CD22 CAR, as provided by cleavage of the CAR, than T cells transduced with a vector encoding the V1 CAR (FIG. 4).

### Example 3

This example demonstrates in vitro activity of a CAR construct based on cytokine production, in accordance with embodiments of the invention, compared to other CARs.

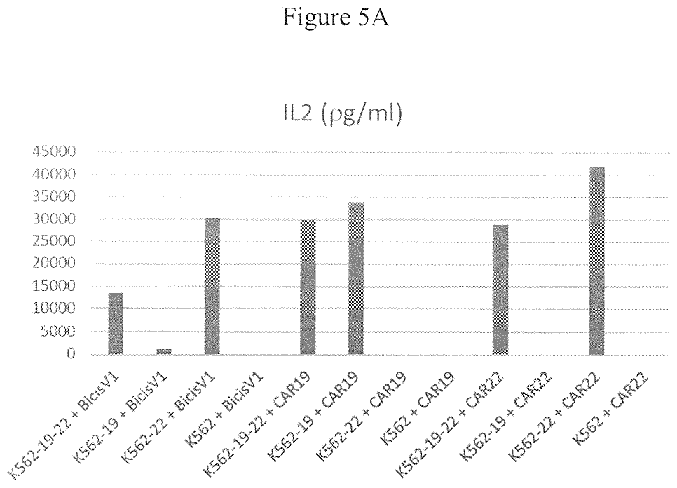

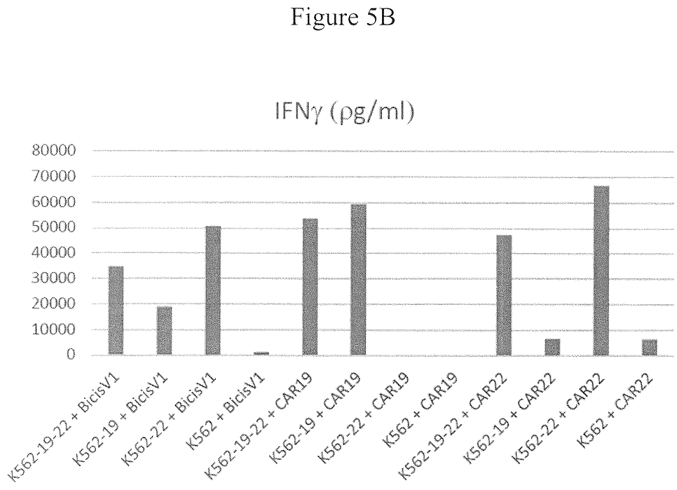

CAR-transduced T cells (1E5) were washed 3 times with 1XPBS and then co-incubated with an equal number of target cells in 200 ml RPMI media in 96-well plates in a 37° C. incubator for 15 to 20 hours. The target cells were K562 expressing CD19 or CD22 or both CD19 and CD22. K562 cells served as the negative control. The cytokine levels of the IL2 and IFNγ in the culture supernatant were measured with an ELISA kit (R&D Systems, Minneapolis, MN, USA). All tests were set in triplicates. V1 CAR T cells made plenty of IL2 and IFNg when co-cultured with CD22 expression target cells, but made only low level of IL2 and IFNg when co-cultured with CD19 expression target cells (FIGS. 5A and 5B).

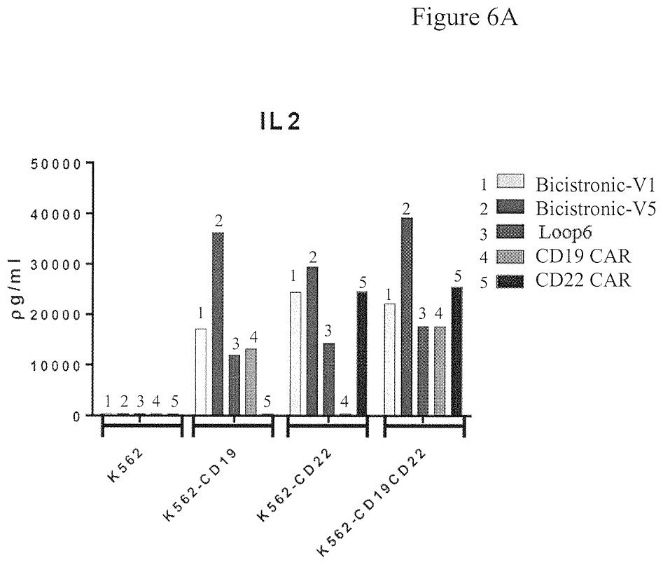

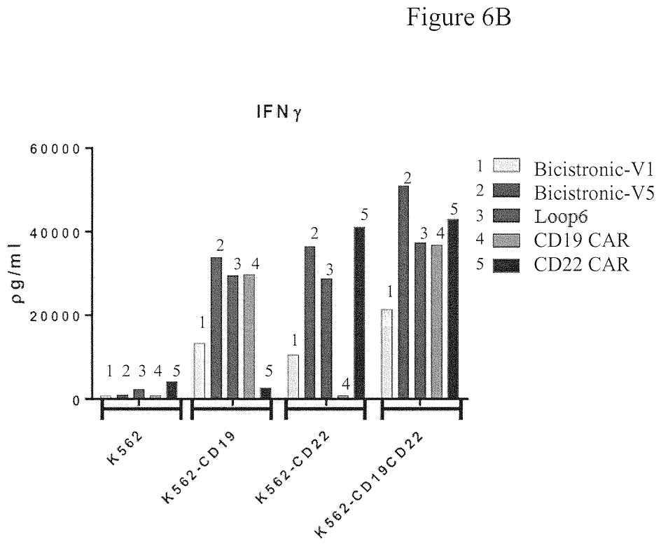

CML cell line K562 was artificially transduced with CD19 or CD22 or both to express the target antigens. K562 cells served as the negative control. 1E5 CAR T cells were washed 3 times and then co-incubated with 1E5 target cells in RPMI media at 37° C. After 14 hours, culture supernatant was harvested and the production of the cytokines was measured with ELISA kits. V5 makes the highest level of both IL2 and IFNg when co-incubated with target antigen expressed on K562 when compared with the bispecific LoopCAR6 and the anti-CD19 and anti-CD22 single CARs. V1 makes plenty of both IL2 and IFNg when co-incubated with target antigen expressed on K562 when compared with the bispecific LoopCAR6 and the anti-CD19 and anti-CD22 single CARs. See FIGS. 6A and 6B.

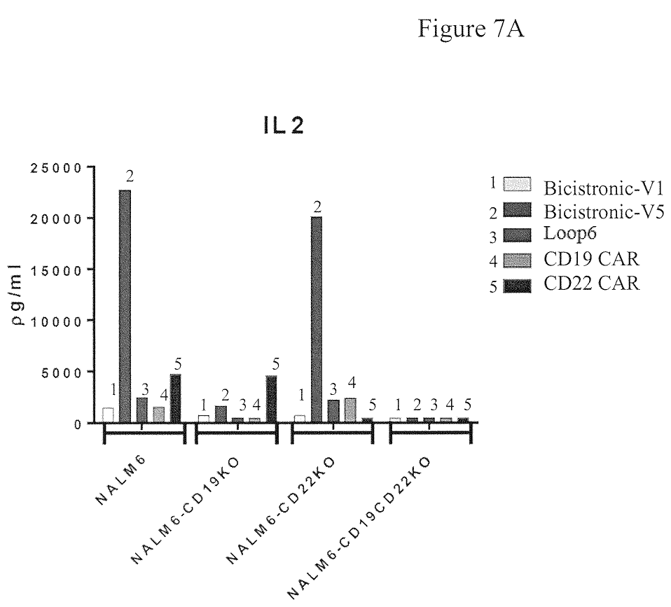

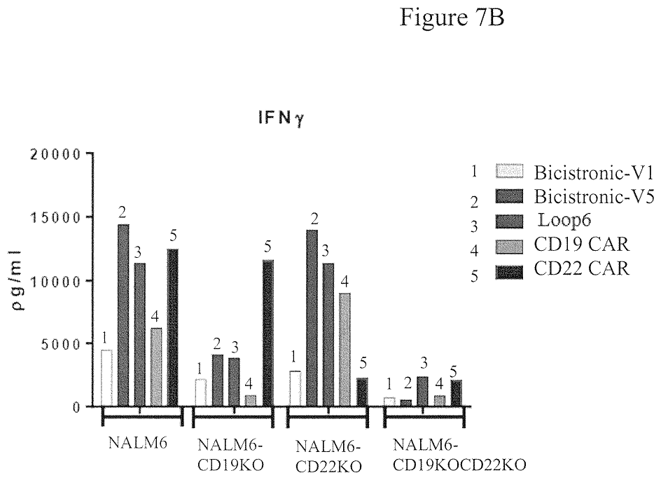

B cell leukemia cell line NALM6 expresses both CD19 and CD22 surface antigens. CD19 or CD22 was knocked out with CRISPR/Cas9 technology to eliminate the expression of these target antigens. NALM6 cells served as the positive control. 1E5 CAR T cells were washed 3 times and then co-incubated with 1E5 target cells in RPMI media at 37° C. After 14 hours, culture supernatant was harvested, and the production of the cytokines was measured with ELISA kits. V5 makes the highest level of both IL2 and IFNg when co-incubated with CD19 expressed on NALM6 when compared with the bispecific LoopCAR6 and the anti-CD19 and anti-CD22 single CARs. V5 makes lower amount of IL2 and IFNg when co-incubated with CD22 expressed on NALM6 when compared with the anti-CD22 single CARs. V1 makes small amount of IL2 and IFNg when co-incubated with target antigen expressed on NALM6 when compared with the V5 CAR. V1 makes comparable amount of IL2 and IFNg with bispecific LoopCAR6 and anti-CD19 and anti-CD22 single CARs. See FIGS. 7A and 7B.

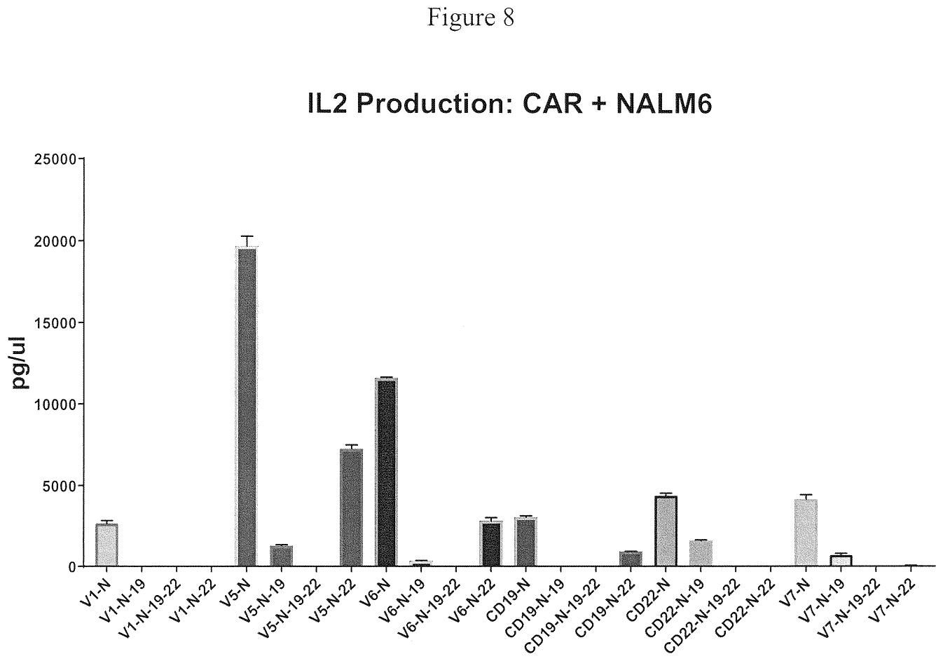

CAR T cells were co-incubated with NALM6 tumor cells for 18 hours, and the levels of IL2 production in the culture supernatant were measured by ELISA. As seen in FIG. 8, Bicis-V5 and Bicis-V6 may have a synergistic effect.

### Example 4

This example demonstrates treatment of a Relapse Leukemic Model using a CAR construct, in accordance with embodiments of the invention, compared to other CARS.

Bioluminescent imaging was used to track leukemia progression in vivo. On day 0, 0.5 million of CD19KO NALM6 cells were mixed with an equal number of CD22KO NALM6 cells and injected into NSG mice. After 3 days, these mice were treated with 3 million CAR T cells or mock T cells.

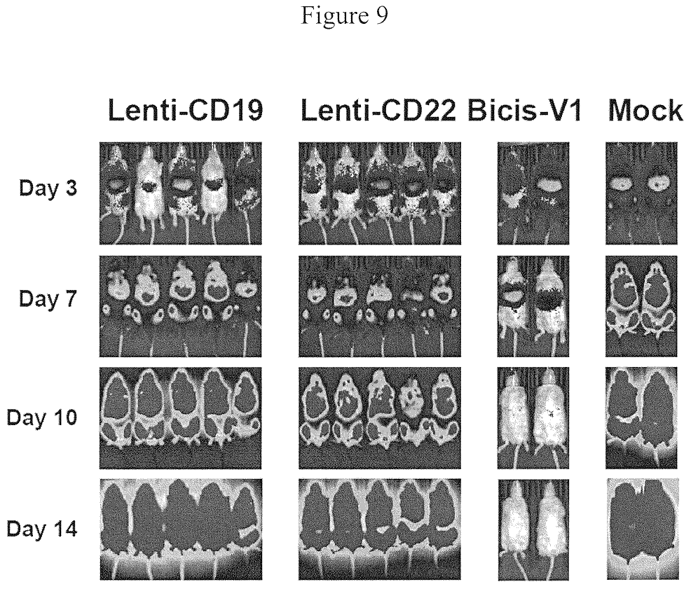

Use of T cells transduced with a vector encoding the V1 CAR appeared to completely eradicate the leukemia while use of T cells transduced with a vector encoding either the single anti-CD19 CAR or the single anti-CD22 CAR failed to do so (FIG. 9).

The mice were euthanized on day 14. The bone marrow (BM) cells were stained with anti-CD19 or anti-CD22 antibody for the detection of leukemia, and also stained with CD22 Fc or anti-idiotype of CD19 for the detection of anti-CD22 CAR or anti-CD19 CAR, respectively.

There was no detectable level of leukemia in mice that were treated with T cells transduced with a vector encoding the V1 CAR, while there was a high tumor burden in mice that were treated with T cells transduced with a vector encoding either the single anti-CD19 CAR or the single anti-CD22 CAR. There was a high tumor burden in mice that were treated with mock T cells. V1 CAR T cells persisted up to day 14 in the BM compartment.

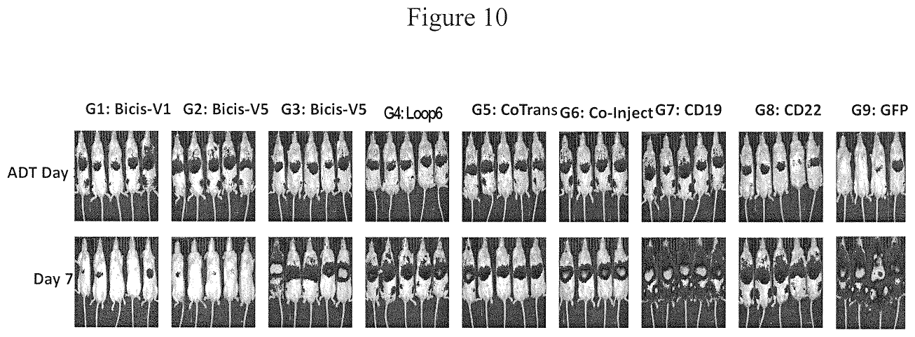

On day 0, NSG mice were injected with mixed leukemia cells (0.1E6 NALM6 and 0.1E6 NALM6-CD19⁻ and 0.1E6 NALM6-CD22⁻). On day 3, mice received 3E6 of CAR T cells in all groups except group 2 (G2) with 6E6 CAR T cells. Mice in group 5 (G5) received CD19 CAR and CD22 CAR co-transduced T cells. Mice in group 6 (G6) received co-administration of 3E6 of CD19 CAR and 3E6 of CD22 CARs. Mice in group 9 (G9) received Lenti-GFP⁺ T cells and served as the negative control. Bioluminescent intensity represents tumor burden. The data suggests that at the same dose level of CAR T cells (3E6), Bicistronic-V1 CAR may be the most potent for reducing the leukemia in this relapse model (FIG. 10).

### Example 5

This example demonstrates treatment of CD19⁻ and CD22⁻ leukemia using a CAR construct, in accordance with embodiments of the invention, compared to other CARs.

Bioluminescent imaging was used to track leukemia progression in vivo. On day 0, 0.5 million of CD19KO NALM6 cells were mixed with an equal number of CD22KO NALM6 cells and injected into NSG mice. After 3 days, these mice were treated with 3 million CAR T cells.

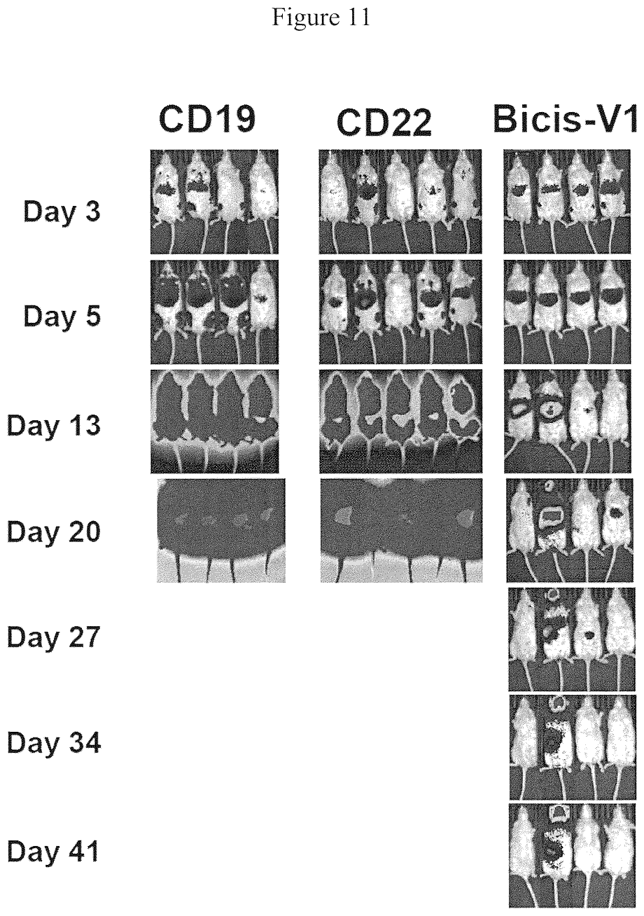

Use of T cells transduced with a vector encoding the V1 CAR almost cleared all of the leukemia, while use of T cells transduced with a vector encoding either the single anti-CD19 CAR or the single anti-CD22 CAR failed to do so (FIG. 11).

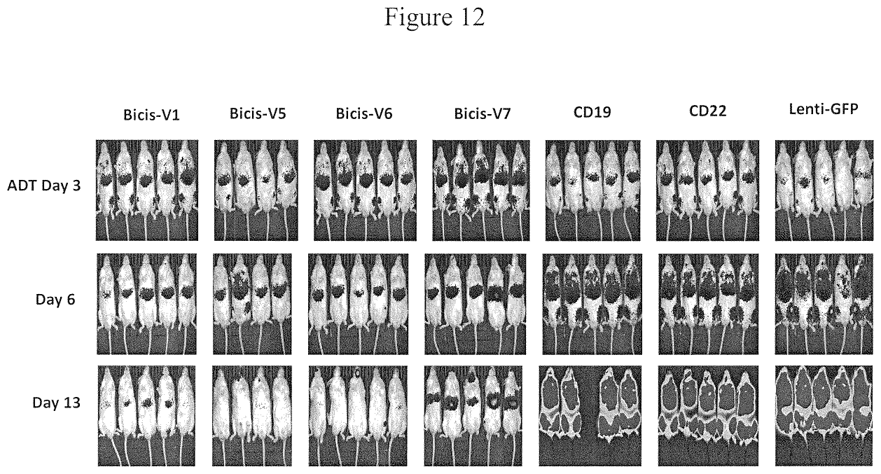

NSG mice were challenged with 1E5 cells of NALM6, 1E5 cells of NALM6-CD19KO and 1E5 CD22KO leukemia cells on day 0, and then received 3E6 of CAR⁺ T cells on day 3. Bioluminescent intensity represent tumor burden. The image indicates that the V1, V5, V6, and V7 CARs are efficient at reducing both CD19⁺CD22⁺ leukemia and also CD19-negative and CD22-negative leukemic cells but the anti-CD19 or anti-CD22 single CARs failed to do so. FIG. 12.

### Example 6

This example demonstrates treatment of leukemia with a CAR construct, in accordance with embodiments of the invention, compared to other CARS.

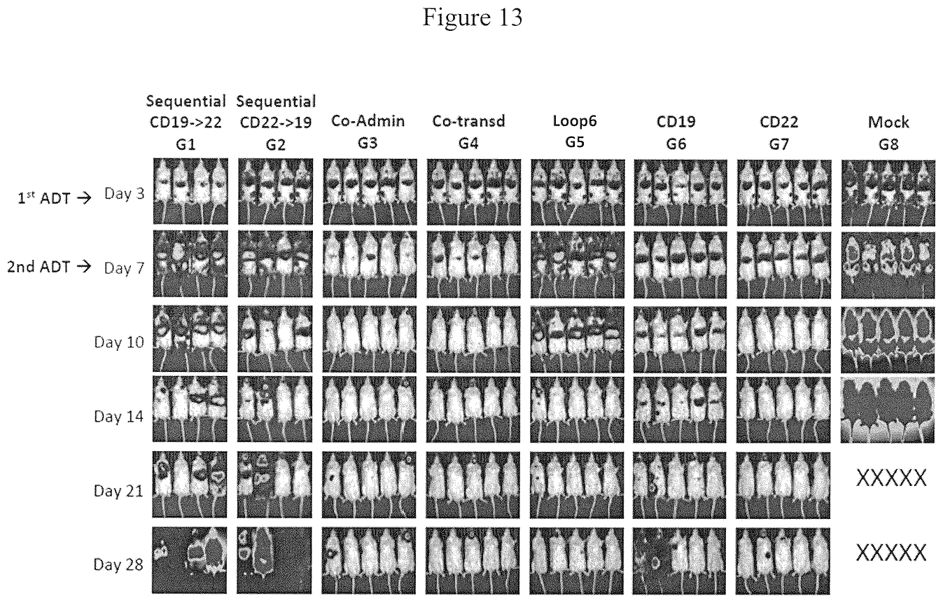

NSG mice were challenged with 1E6 of NALM6 leukemia cells on day 0. Mice in group 1 and group 2 (see FIG. 13 for mouse groups) received sequential treatment with 1E6 T cells transduced with a vector encoding the single anti-CD19 CAR or single anti-CD22 on day 3 and 3E6 T cells transduced with a vector encoding the other single CAR on day 7. Mice in group 3 received co-administration of a total of 6E6 CAR+ T cells with 3E6 T cells transduced with a vector encoding the single anti-CD19 CAR and 3E6 T cells transduced with a vector encoding the single anti-CD22 CAR on day 3. Mice in groups 4 to 7 received 3E6 of T cells transduced with a vector encoding the CARs indicated in FIG. 13 on day 3. Mice in group 4 received almost 10E6 of total CAR+ T cells due to the low expression on the co-transduced T cells. FIG. 13 demonstrates that simultaneously targeting of both CD19 and CD22 is better than sequential treatment with single targeting CARs.

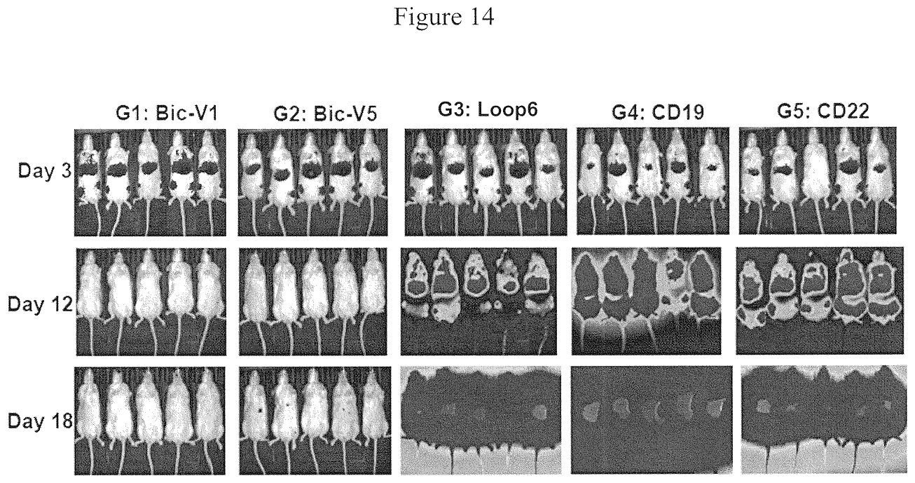

NSG mice were challenged with 0.25E6 of NALM6-CD19KO and 0.25E6 CD22KO leukemia cells on day 0. On day 3, NSG mice were injected with 3E6 CAR+ T cells. Use of T cells transduced with a vector encoding the V1 CAR and use of T cells transduced with a vector encoding the V5 CAR appear to completely eradicate both CD19⁻ and CD22⁻ leukemic cells (FIG. 14).

### Example 7

This example demonstrates bispecific CARs, in accordance with embodiments of the invention.

### Human Leukemia Samples

Patient samples were screened for antigen expression via a NCI IRB approved screening protocol. Human ALL samples for xenograft generation were collected and stored after informed consent to an National Cancer Institute (NCI)-IRB approved tissue acquisition protocol. All research specimens from human subjects were obtained with informed consent in accordance with the Declaration of Helsinki.

### Cell Lines and Culture Conditions

The following leukemia cell lines were used: the erythroleukemia K562-CD22 (transduced with human CD22, GeneCopoeia, Cat: EX-Z9364-Lv151), K562-CD19 (transduced with human CD19), K562-CD19CD22 (transduced with both human CD19 and CD22), non-transduced K562 as a negative control; the B cell acute lymphoblastic leukemia lines NALM6, NALM6-GL (transduced with GFP and Luciferase) and REH-TSLPR-GL (Qin et al., Blood, 126:629-39 (2015), incorporated by reference). Cell lines were cultured in media supplemented with 10% heat-inactivated FBS, 10 mM HEPES, 100 U/mL penicillin, 100 μg/mL streptomycin, and 2 mM L-glutamine (Invitrogen). The Lenti-X 293T lenti packaging cell line (Clontech. Cat #632180) was cultured in DMEM (Invitrogen) media.

### Creation of the CD19neg and CD22neg Leukemia Relapse Model

CRISPR Cas9 technology was used to edit Nalm6 to generate NALM6-CD19neg-GL (CRISPR CD19 on exon 3), NALM6-CD22neg-GL (CRISPR CD22 on exon 6). Lentiviral vectors for CRISPR/Cas9 gene editing of CD19 or CD22 on NALM6 was previously described (Fry et al., Nat. Med., 24:20-28 (2018), incorporated by reference). Briefly, guide-RNAs were optimized by http://crispr.mit.edu/, cloned into LentiCRISPR v2 plasmid (Addgene Plasmid 52,961). Plasmids were then co-transfected with packaging plasmids and transformed into HEK293T cells. Two days later, supernatants were harvested, filtered, and concentrated. For viral transduction, 105 NALM6 cells were incubated with 10 ml of concentrated viral supernatant for 2 days, followed by expansion in RPMI media. Cell phenotype was assessed by flow cytometry, followed by sorting of cells with phenotypic alterations and single-cell cloning. Sequencing was performed on single-cell clones to confirm genotypic alterations.

### CAR Lentiviral Vector Production and T Cell Transduction

Bivalent CAR constructs were designed and synthesized followed by cloning into lentiviral transfer plasmids. Bivalent CAR-encoding lentiviral vectors were produced by transient transfection of the Lenti-X 293T lenti packaging cell line. Briefly, lenti-X 293T cells were plated into poly-D lysine coated 15-cm plates (BD Biosciences). The following day, Lenti-X 293T cells were transfected using lipofectamine 3000 (Thermo Fisher Scientific) with plasmids encoding the CAR construct along with packaging and envelope vectors (pMDLg/pRRE, pMD-2G, and pRSV-Rev). Lentiviral supernatants were harvested at 24 and 48 hours post-transfection, centrifuged at 3000 RPM for 10 minutes to remove cell debris, frozen on dry ice and stored at −80° C. Human PBMCs from normal donors were obtained with an NIH-approved protocol and activated with a 1:3 ratio of CD3/CD28 microbeads (Dynabeads Human T-Expander CD3/CD28, Thermo Fisher Scientific, Cat #11141D) in AIM-V media containing 40 IU/mL recombinant IL-2 and 5% FBS for 24 hours. Activated T cells were resuspended at 2 million cells per 2 mL of lentiviral supernatant plus 1 mL of fresh AIM-V media with protamine sulfate at the final concentration of 10 μg/mL and 100 IU/mL IL-2 in 6-well plates. Plates were centrifuged at 1000×g for 2 hours at 32° C. and incubated overnight at 37° C. A second transduction was performed on the following day by repeating the same transduction procedure described above. The CD3/CD28 beads were removed on the third day following transduction, and the cells were cultured at 300,000 cells/mL in AIM-V containing 100 IU/mL IL2 with fresh IL2-containing media added every 2-3 days until harvest on day 8 or 9.

### Flow Cytometry Analysis

Surface expression of CD22 CAR-transduced T cells was determined by flow cytometry using a CD22-Fc (R&D Systems) followed by incubation with PE-F (ab) 2 or APC-F (ab) 2 specific for human IgG-Fc (Jackson ImmunoResearch Laboratories). Surface expression of CD19 CAR-transduced T cells was detected with anti-CD19 Idiotype or Recombinant Human CD19 Fc Chimera Protein (R&D Systems) conjugated with APC by using Lightning-Link APC Antibody Labeling Kit (Novus Biologicals). Expression of bivalent CARs was assessed using a combination of both detection reagents as indicated for individual figures. Expression of CD19 and CD22 on B-ALL lines were detected using the following anti-human antibodies: CD45-PerCP-Cy5.5 (eBioscience), CD19-Pacific Blue, CD19-APC-Cy7, CD10-PE-Cy7, and CD22-PE (Biolegend). T cells were characterized with the following antibodies: CD3-APC-Cy7, CD4-Pacific Blue, and CD8a-PE-Cy7 (BioLegend).

### Incucyte Cytotoxicity Assay

5E4 of Target tumor cells in 100 ul of RPMI media were loaded into a 96-well plate (Corning® BioCoat™ Poly-L-Lysine 96-Well Clear TC-Treated Flat Bottom Assay Plate). An equal amount of CAR T cells were added into the designated well on the following day. An apoptosis marker (Essen BioScience) was diluted in 100 ul PBS and 1 ul of the diluent was added into each well. The plate was scanned for the GFP fluorescent expression to monitor apoptosis GFP-positive cell disappearance using an IncuCyte ZOOM® system every 30 minutes in a duration of 40 hours. The percentage of cell killing at each time point determined relative to baseline.

### Analysis of Cytokine Production

Target tumor cells and transduced CAR positive T cells were washed 3 times with PBS and resuspend in RPMI at 1E6/ml. 100 ul of tumor cell suspension and 100 ul of CAR T cells suspension was loaded into each well of 96-well plate with T cell only and tumor cell only controls in duplicates or triplicates. After 18 hours at 37° C. incubator a culture supernatant was harvested for detection of the cytokines using either ELISA (R&D Biosciences) or a multiplex assay (Meso Scale Discovery).

### In Vivo Studies

Animal experiments were carried out under protocols approved by the NCI Bethesda Animal Care and Use Committee. B-ALL cell lines and the xenografted human B-ALL specimens were IV injected into NSG mice (NOD.Cg-PrkdcscidIl2rgtm1Wjl/SzJ, Jakcson Laboratories). For luciferase-expressing lines, leukemia was detected using the Xenogen IVIS Lumina (Caliper Life Sciences). Mice were injected intraperitoneally with 3 mg D-luciferin (Caliper Life Sciences) and were imaged 4 minutes later with an exposure time of 30 sec for NALM6 and 2 minutes for PDXs. Living Image Version 4.1 software (Caliper Life Sciences) was used to analyze the bioluminescent signal flux for each mouse as photons/s/cm²/sr. Leukemia burden in non-luciferase expressing xenografts was measured by flow cytometry of peripheral blood, bone marrow, and spleen.

### Patient-Derived Xenografts

The following primary samples were used for the generation of PDX models: CD19 ALL and the CD19⁺CD22^{dim} (de novo relapse specimens ALL_H0113_post22_r (CAR3), ALL_H0090_post19_pd (HMB15). PDXs were created by injecting 1E6 to 10E6 of the patient ALL cells intravenously into NSG mice (NOD scid gamma, NOD.Cg-Prkdescid Il2rgtm1Wjl/SzJ; Jackson ImmunoResearch Laboratories). After 2 successful passages PDX lines were transduced with lenti-GFP-Luc virus and sorted for high expression of GFP and luciferase after the first and second passages. GFP-transduced PDX leukemia in vivo burden was assessed by weekly fluorescence imaging, and animals were treated with CAR T cells via tail vein injection once the human ALL was detectable by florescence imaging. Elutriated human lymphocytes from healthy donors were obtained from the Department of Transfusion Medicine at the National Institutes of Health (NIH) Clinical Center under an IRB-approved protocol. The human lymphocytes were cultured in AIM-V media.

### Genomic Analysis of PDX Models

Nucleic acid extractions were performed on viably cryopreserved samples using Qiagen AllPrep micro kits per the manufactures protocol (Qiagen). DNA and RNA were quantified and assessed for quality using an Agilent 2100 BioAnalyzer. Poly-adenylated RNA libraries were generated and sequenced using TruSeq 4.0 chemistry on a Hiseq2500 (Illumina) platform. Whole exome data was generated using Agilent SureSelect XT Human All Exon V5 and TruSeq V4 chemistry and sequenced to a median of 100× coverage using HiSeq 2500 (Illumina).

Whole-exome and RNA-sequencing data was mapped and analyzed using the CCR Collaborative Bioinformatics (CCBR) pipeline (https://bioinformatics.cancer.gov). Reads were aligned to reference genome Hg19. Somatic variant calling was performed using MuTect20 and copy number alterations were analyzed using Nexus Copy Number Discovery Edition #9 (BioDiscovery). The integrity of the CD19 and CD22 gene was further interrogated by manual inspection using Integrative Genome Viewer (IGV). RNA sequencing reads for each sample were trimmed of their adapters and low quality bases using Trimmomatic software and alignment with reference human Hg 38 and Genecode V24 transcripts using STAR software.

### Statistical Analysis

Statistics analysis were performed using Prism 7.0 software. Statistical significance was calculated using Mann Whitney test for patient CD19 and CD22 analyses.

### Heterogeneous and Dynamic Expression of CD19 and CD22 on Pre-B ALL.

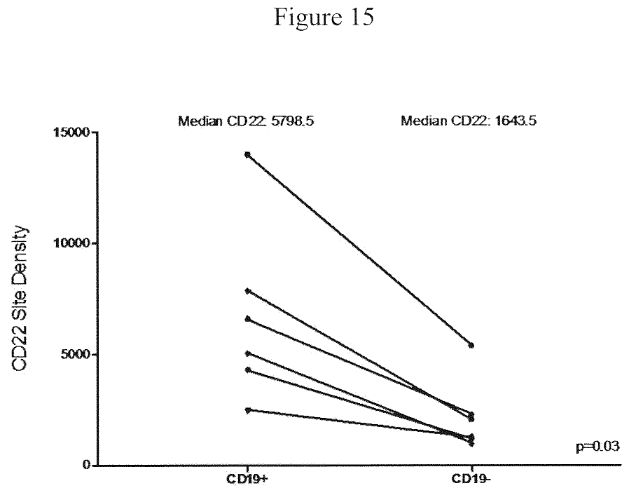

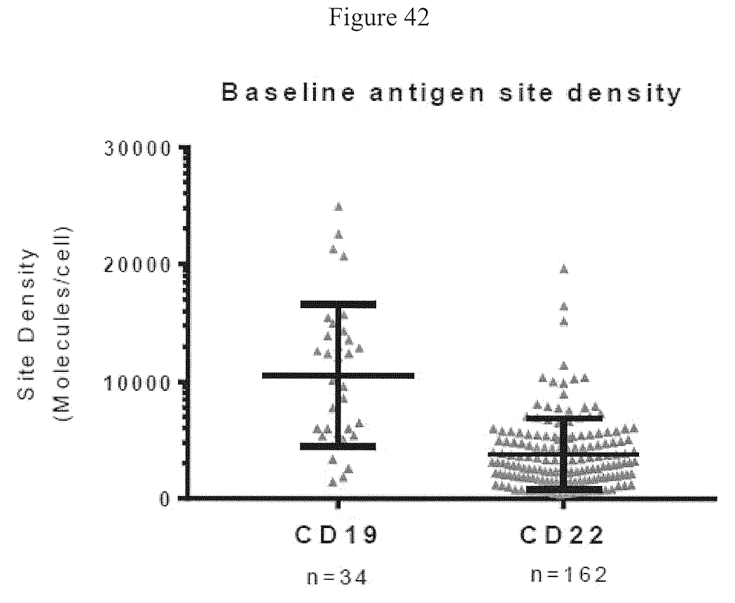

Patient samples, primarily derived from patients with multiply relapsed disease, were evaluated for CD19 and CD22 expression. There was a broad range in expression of CD19 and CD22 prior to administration of immunotherapy (FIG. 42). CD19 epitope loss has been well-described following CD19-targeted immunotherapy. In a matched paired analysis, CD22 expression was evaluated in patients prior to and after loss of CD19 and demonstrated a consistent decrease in CD22 expression associated with CD19 loss, suggesting that single antigen loss may also broadly modulate antigen expression (FIG. 15). These results illustrate the challenges associated with single-antigen targeted immunotherapy.

Simultaneous targeting of both CD19 and CD22 is superior to sequential treatment in prevent relapse or disease progression of antigen loss-relapse models.

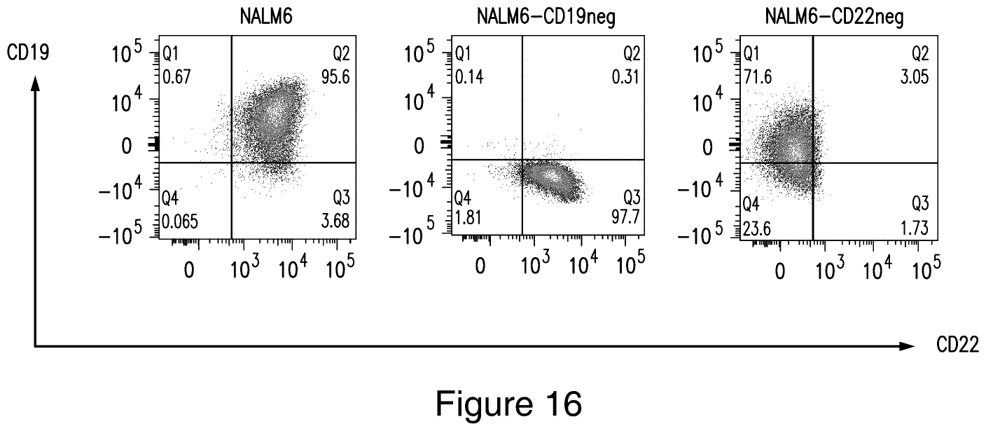

To model the CD19 and CD22 relapse phenomenon seen in clinical trials, CRISPR/Cas9 gene editing was used to delete CD19 and/or CD22 from the pre-B ALL cell line NALM6 cell (FIG. 16) with certain data provided in Table 17.

TABLE 17

| | Diff in GeoMean CD22 | Diff in GeoMean CD19 |
|---|---|---|
| NALM6-CD19neg | 1909 | 0 |
| NALM6-CD22neg | 0 | 5779 |
| NALM6-GL | 3670 | 10733 |

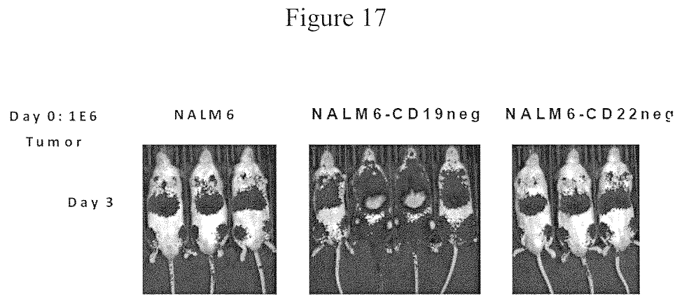

After single cell cloning to ensure stability, both CRISPR-edited NALM6 lines and parental NALM6 all demonstrated disease progression when engrafted in NSG mice despite deletion of either B cell receptor-associated gene and loss of corresponding surface protein expression (FIG. 17).

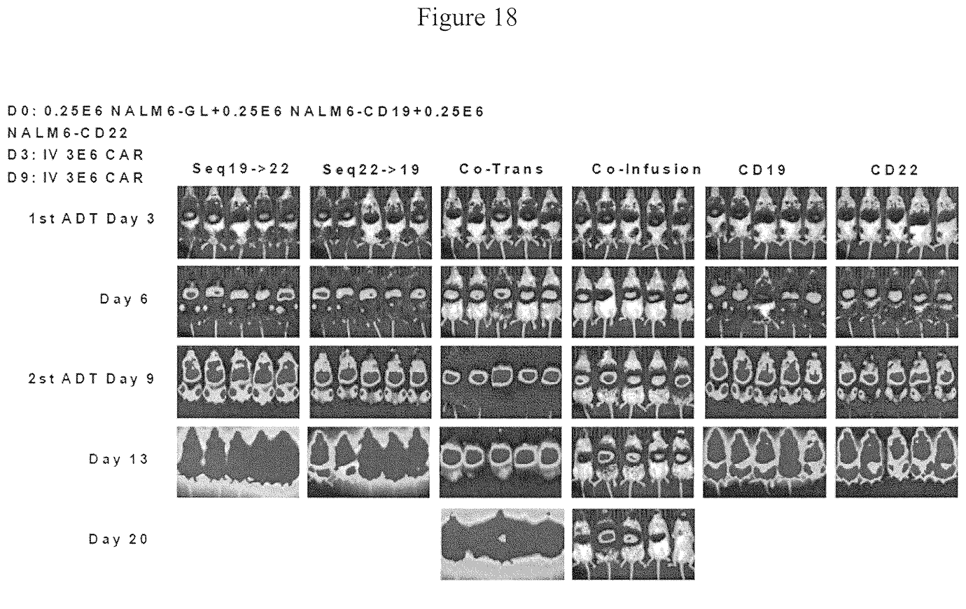

One approach to exert immunotherapeutic pressure on two antigens is via sequential infusion of anti-CD19-CAR T cells followed by anti-CD22 CAR T-cells or vice versa. To test this strategy, mice were injected with a mixture of CD19neg, CD22neg and parental NALM6 (CD19pos/CD22pos) ALL to simulate antigen-negative relapse. Administration of single antigen-specific CAR T cells resulted in recurrence of leukemia not expressing the targeted antigen, validating the relapse model (FIG. 18). Surprisingly, sequential infusion of curative doses of anti-CD19 and anti-CD22 CART separated by 6 days did not prevent ALL progression. Importantly, the relapse phenotype demonstrated reduced efficacy of the second CAR infusion. Simultaneous administration (co-infusion) of both anti-CD19 and anti-CD22 targeted CART was superior to sequential infusion but resulted in progression of CD19neg ALL still expressing CD22, suggesting that the anti-CD19 CAR may dominate.

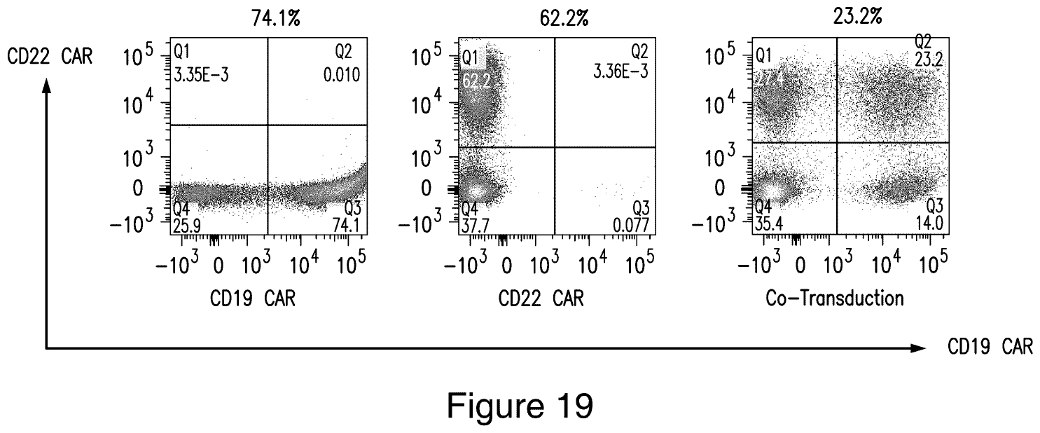

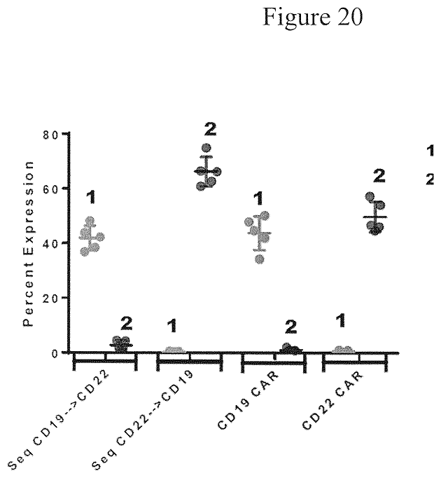

Based on the apparent dominance of the anti-CD19 CAR when co-infused with the CD22 CAR, the next step was to introduce both the anti-CD19 and anti-CD22 CAR into the same T cell through co-transduction, generating a pool of T cells containing dually specific CAR T cells. However, co-transduction efficiency was consistently low, yielding only a quarter of the total T cell product expressing both anti-CD19 and anti-CD22 CAR (FIG. 19). Furthermore, the relapse phenotype (CD22+CD19neg and CD22negCD19neg, FIG. 20) suggests that again, the anti-CD19 CAR T cell may dominate when administered with T cells expressing both anti-CARs or the anti-CD22 CAR alone. Thus, based on the inefficiency of gene transfer by two vectors, the technical challenges and costs associated with managing two vectors as well as the possibility that inclusion of two single CAR expressing T cell may impair expansion of the dually specific T cell population, approaches were pursued to introduce dual-specificity from the same vector.

Development of the Bivalent CARs with Tandem Sequencing of scFv.

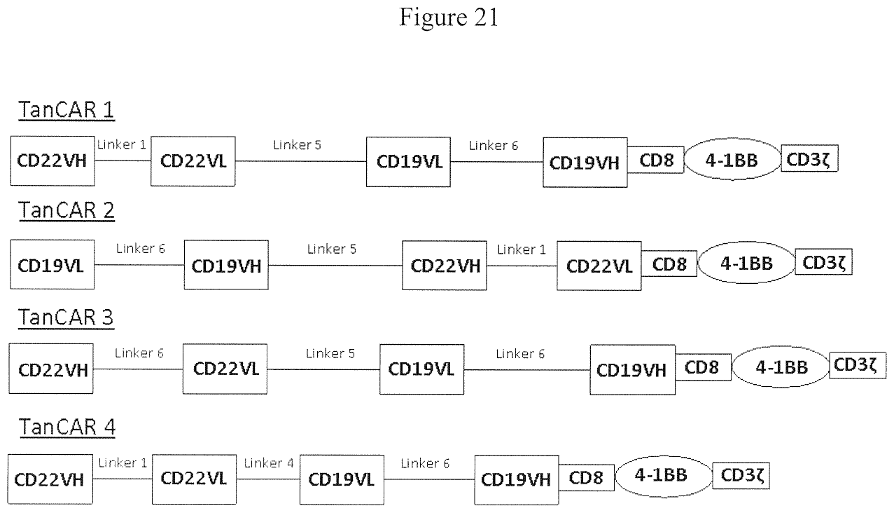

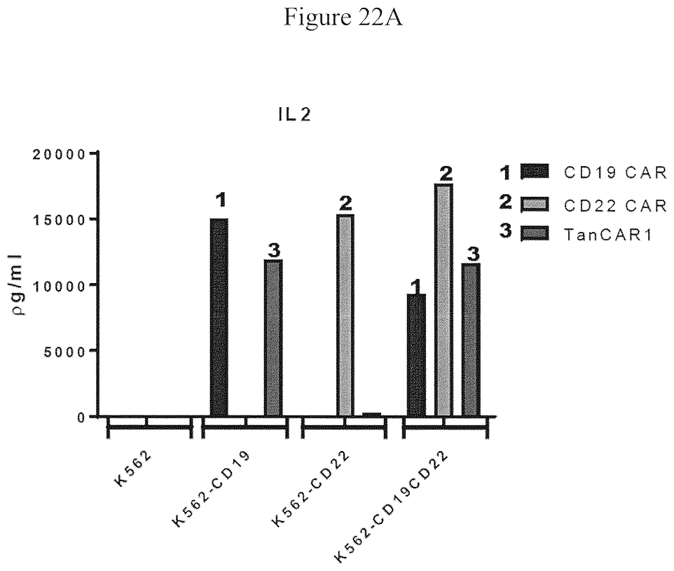

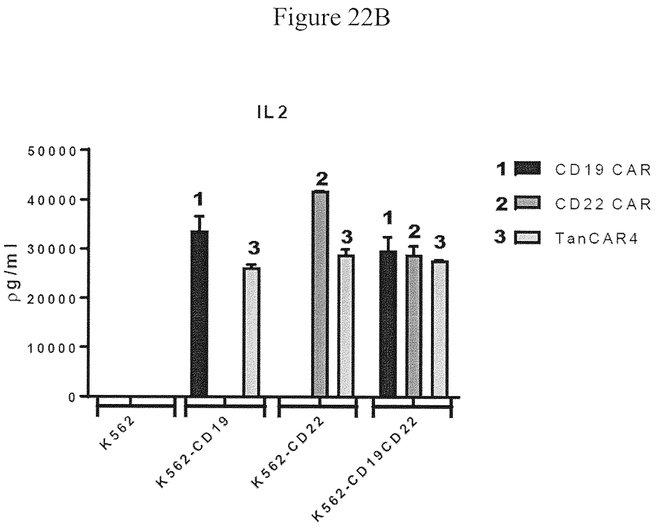

Bivalent CARs were generated by coupling two different scFv domains into a single CAR construct. The approach undertaken in constructing anti-CD19×anti-CD22 CARs was to place the heavy (V_{H}) and light chains (V_{L}) for each scFv (FMC63 for CD19 and m971 for CD22) in sequential order to make tandem CARs (TanCAR) as depicted in FIG. 21. For TanCAR1, the original linkers between the V_{H} and V_{L} from each single CAR were maintained and connected the two scFvs using a G(S)4×5 linker, a format that could be detected at a comparable level to single antigen targeted CARs on the cell surface following transduction. TanCAR1 is described in International Patent Publication No. WO 2016/149578. Importantly, all CAR-expressing T cells could be detected using anti-CD22 Fc fusion and anti-FMC63 idiotype. For TanCAR2 (SEQ ID NO: 63), the order of anti-CD19 and anti-CD22 scFv's were flipped, resulting in much lower detection on the surface. Despite good surface detection of TanCAR1 and comparable levels of IL2 production compared to mono-valent CD19 CART against CD19pos/CD22neg ALL, IL-2 production was extremely low when co-incubated with CD9neg/CD22pos ALL (FIGS. 22A and 22B). Given the extremely short linker (G4S) between the anti-CD22 V_{H} and V_{L}, TanCAR3 (SEQ ID NO: 64) was constructed with increased linker length within the CD22 scFv, a format that abolished CD22 Fc and anti-idiotype binding (FIG. 21). For TanCAR4 (SEQ ID NO: 65), the short parental linker for the anti-CD22 scFv was maintained but the length of the linker was reduced between the anti-CD19 and anti-CD22 scFv. This resulted in CAR surface expression (anti-CD22 Fc and anti-FMC63 idiotype binding) and enhanced CD22-directed functionality compared to TanCAR1 as measured by the IL2 production against CD19−/CD22+ ALL (FIGS. 22A and 22B).

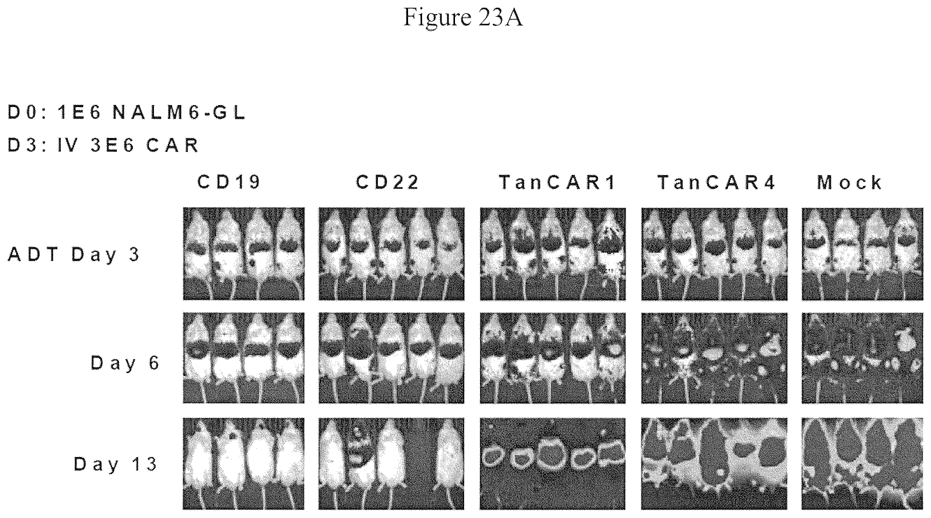

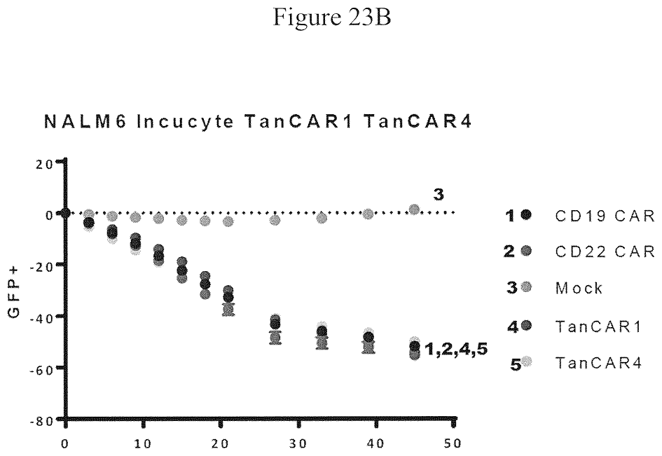

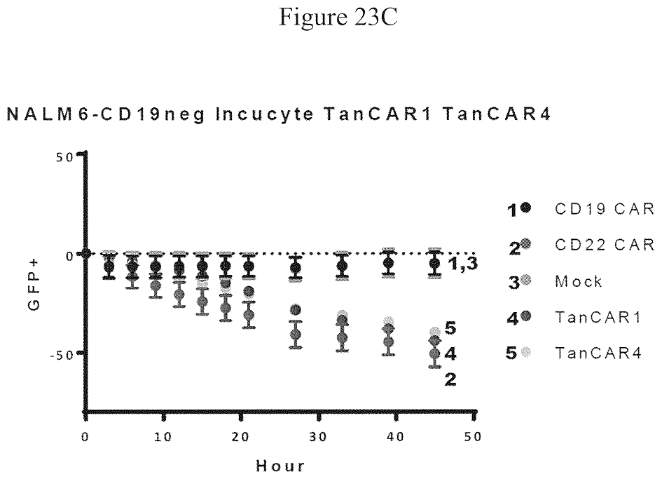

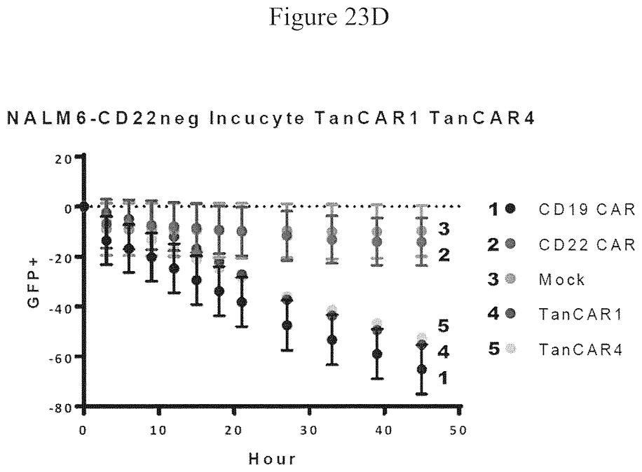

Cytotoxicity of TanCAR1 and TanCAR4 were further evaluated demonstrating comparable activity to CD19 and CD22 monovalent CARs. Despite in vitro activity, neither TanCAR1 nor TanCAR4 fully eradicated CD19posCD22pos ALL in vivo (FIGS. 23A-23D).

Development of the Bivalent CARs with Alternative Sequence of scFv Resulting in Loop Structure.

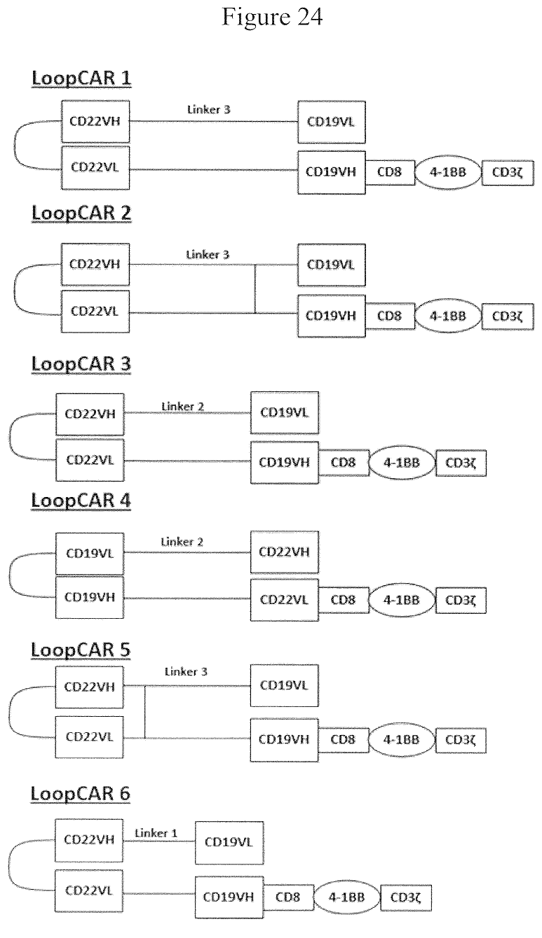

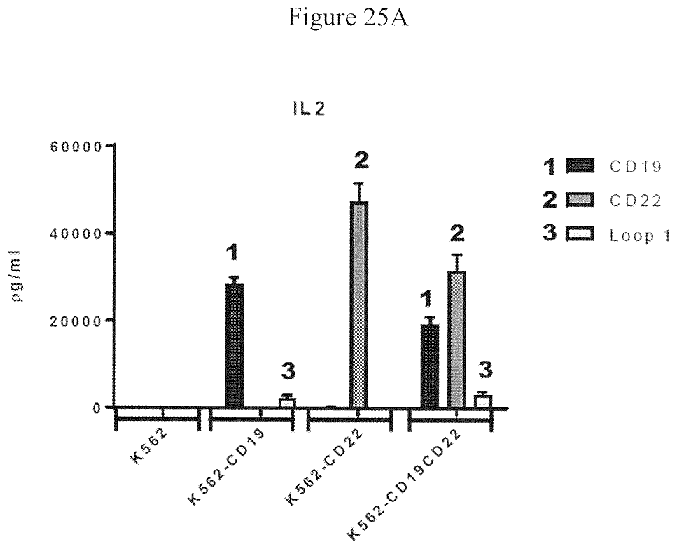

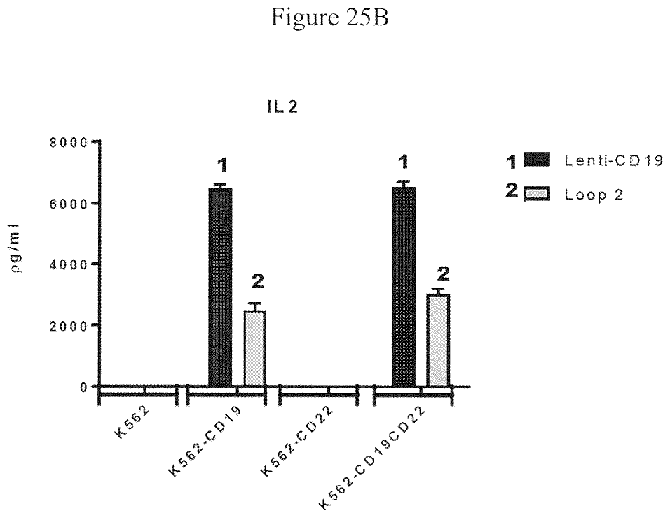

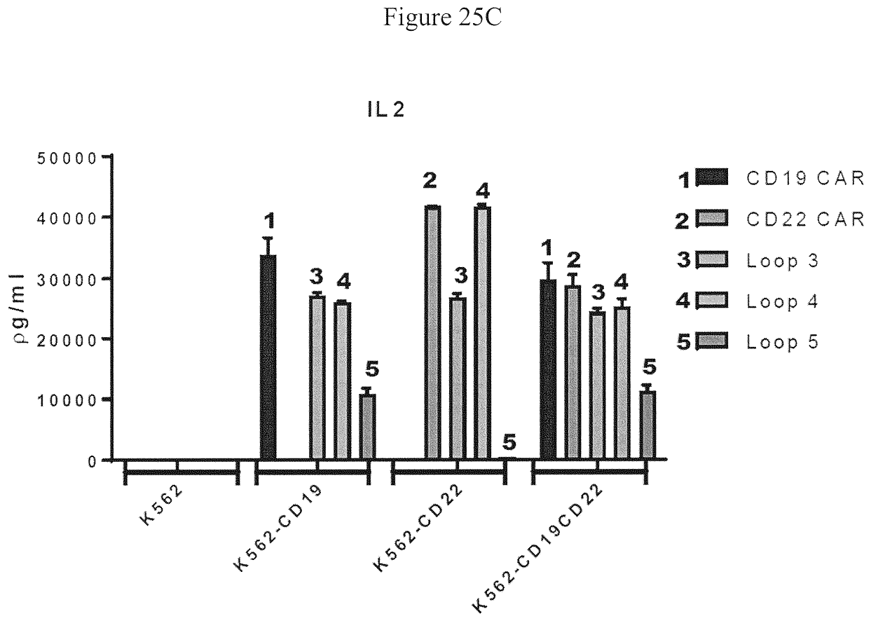

A series of bivalent CAR constructs were constructed (FIG. 24). Loop CAR1 (SEQ ID NO: 66) was constructed with the anti-CD22 scFv (maintaining the short linker) between the VH and VL of the anti-CD19 scFv, a format that could only be detected at low percentages on cell surface. For LoopCAR2 (SEQ ID NO: 67), the length of the linker was increased between anti-CD22 scFv in an attempt to facilitate folding of the loop structure, and the amino acid structure of the linker was slightly modified between the anti-CD19 variable chains and the anti-CD22 scFv to facilitate disulfide bond formation. This improved CAR surface detection. LoopCAR1 failed to generate IL-2 production against either CD19 or CD22. Despite improved surface detection and some IL-2 production against CD19, LoopCAR2 did not generate detectable IL-2 against CD22 antigen. See FIGS. 25A and 25B.

LoopCAR3 (SEQ ID NO: 68) was further modified to reduce the length of the linker between the anti-CD19 heavy chains and the anti-CD22 scFv, and the slightly longer linker was maintained between the V_{H} and V_{L} introduced in Loop2, resulting in improved IL-2 production against CD19neg/CD22pos ALL. For the next series of constructs, the anti-CD19 scFv was placed in a membrane distal location and between the variable chains of the anti-CD22 scFv. In LoopCAR4 (SEQ ID NO: 69), the linker between anti-CD19 scFv and the anti-CD22 scFv variable chains introduced in LoopCAR3 was maintained, resulting in high levels of CAR detection and superior IL2 production compared to any of the previous formats, suggesting the anti-CD22 scFv membrane proximal location may be optimal. Given that IL-2 production against CD19neg/CD22pos ALL was still inferior to the anti-CD22 monovalent CAR, LoopCAR5 (SEQ ID NO: 70) was modified to favor disulfide bond formation, a structure that did not improve cytokine production. See FIG. 25C.

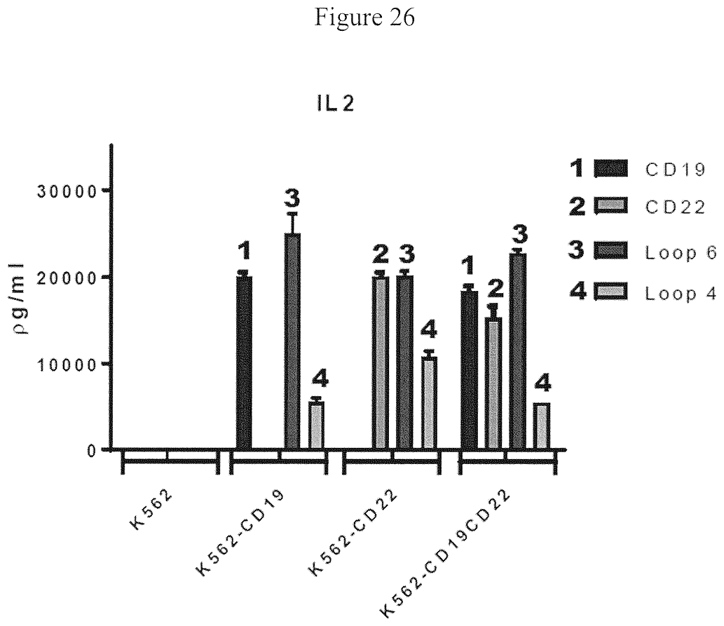

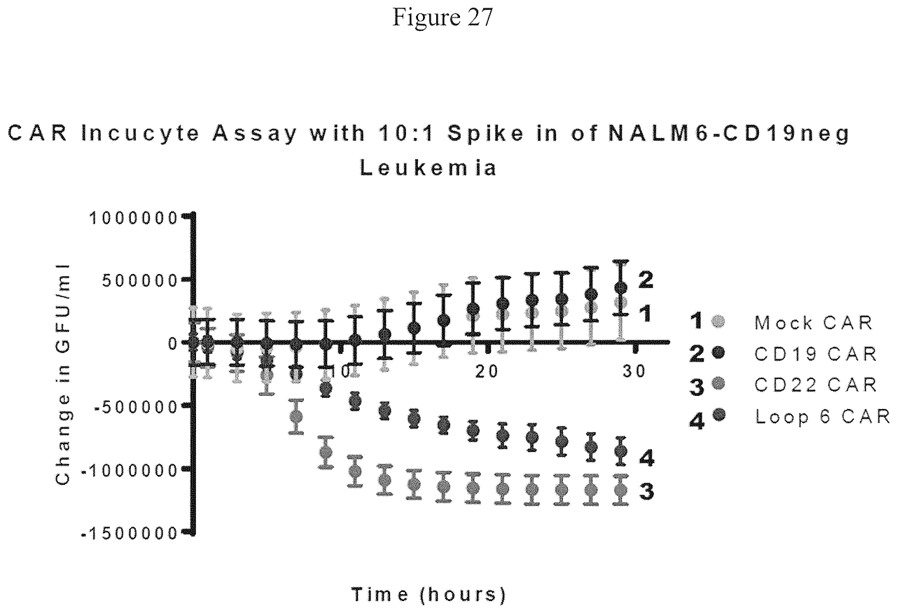

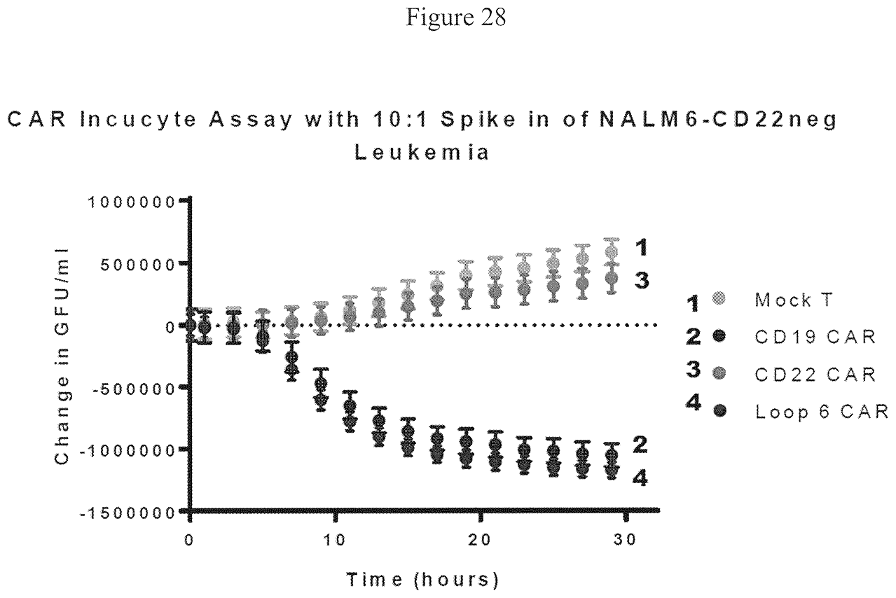

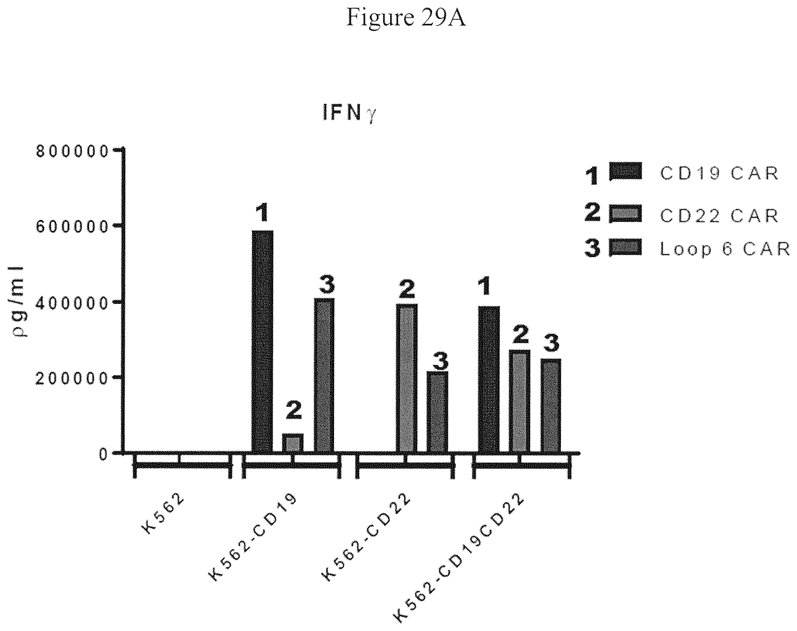

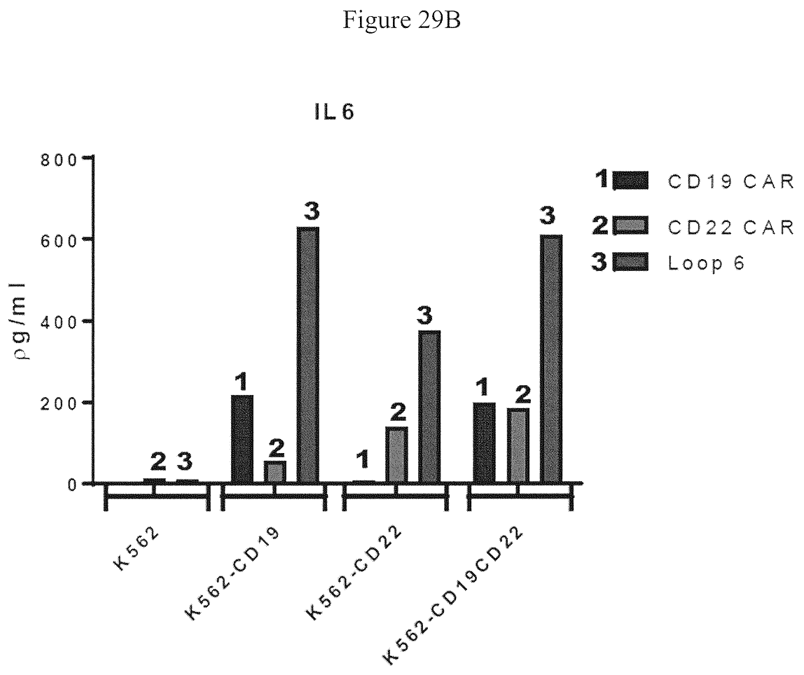

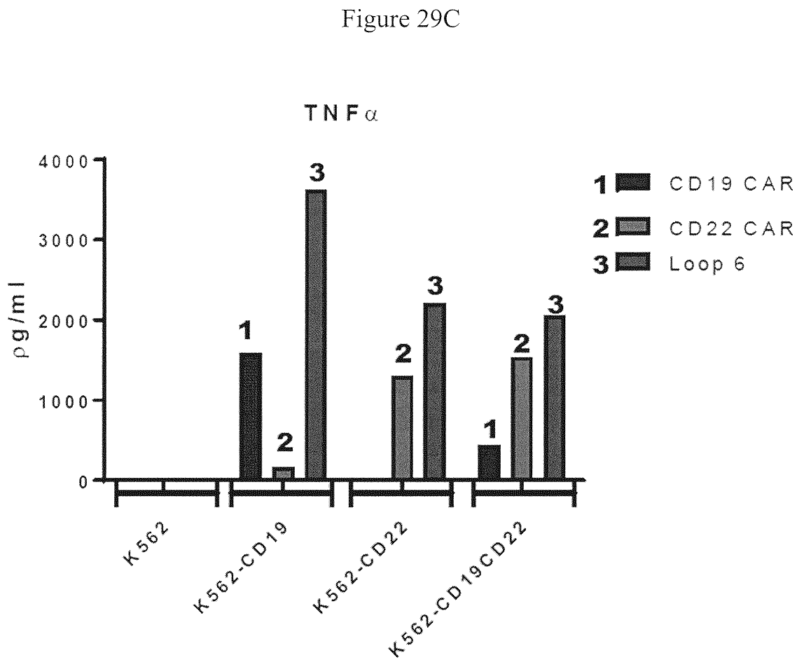

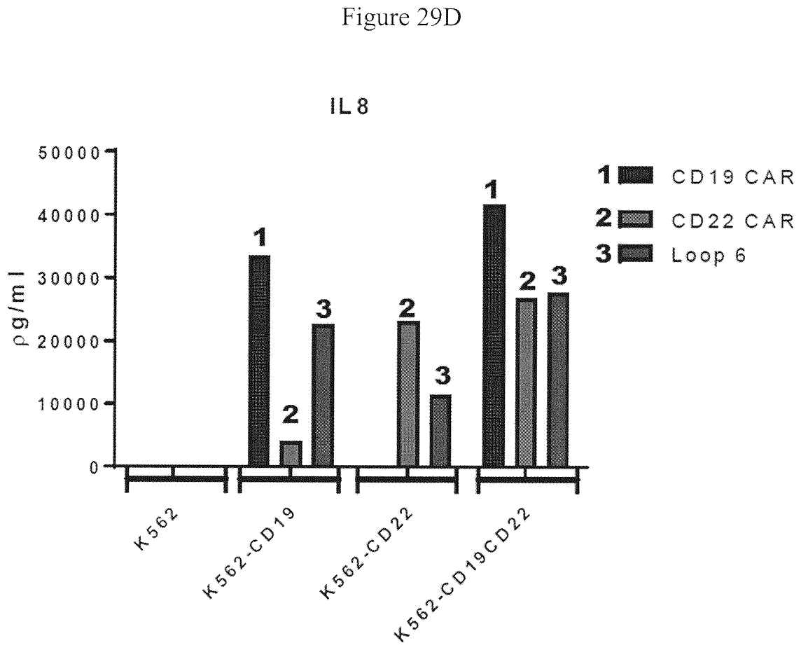

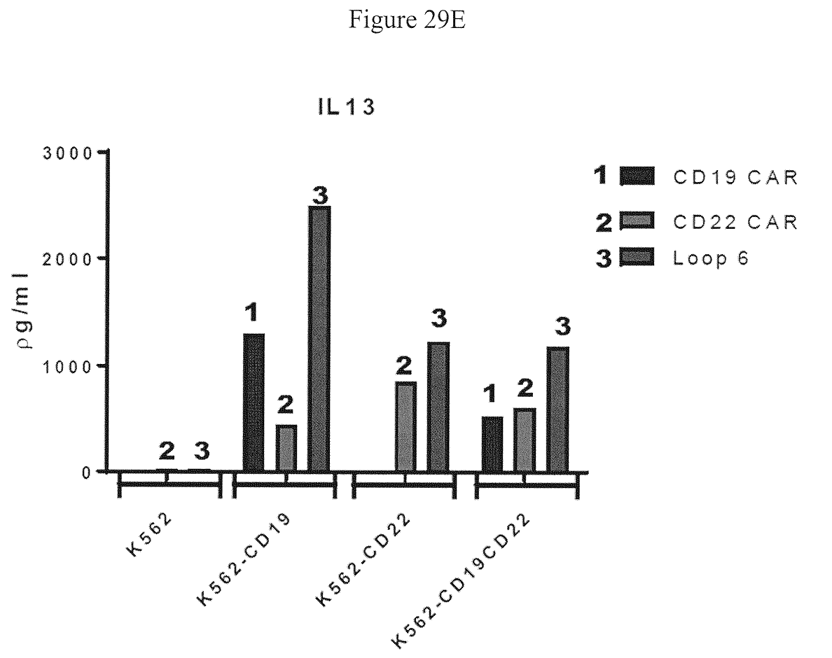

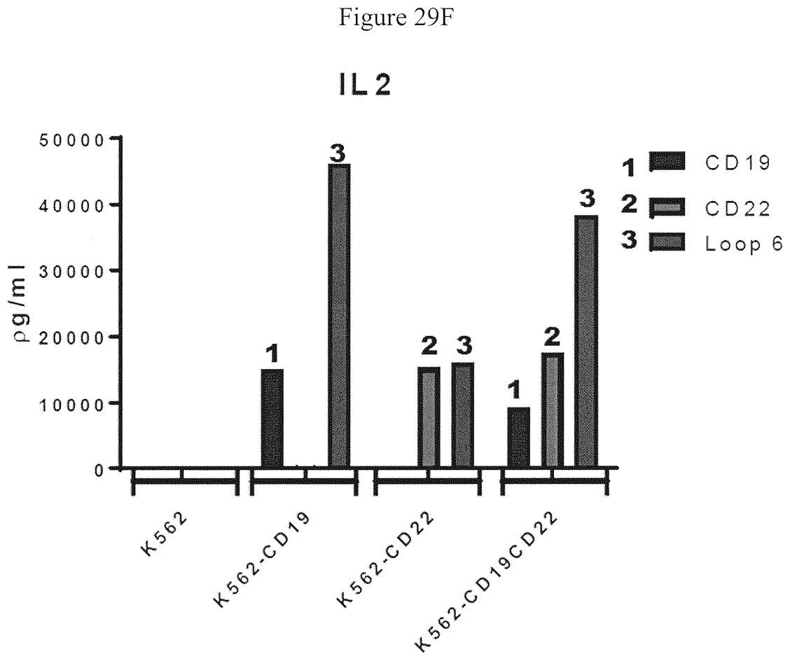

Finally, in LoopCAR6 (SEQ ID NO: 61), a shorter linker was incorporated between anti-CD19 scFv and anti-CD22 variable chains, which dramatically improved both CAR detection and IL-2 production against both CD19pos/CD22neg and CD19neg/CD22pos ALL (FIG. 26) as well as in vitro killing of single-antigen expressing ALL (FIGS. 27 and 28). Of note, the kinetics of killing of the CD19neg ALL by LoopCAR6 expressing T cells compared to anti-CD22 CAR expressing T cells suggested slightly less potency against CD22. LoopCAR6 produced multiple cytokines in response to both CD19 and CD22 (FIGS. 29A-29F) further confirming the potency and polyfunctionality of LoopCAR6 expressing T cells. Thus, the loop design may be optimal for bivalent CARs incorporating CD19×CD22 specificity, likely due to challenges maintaining CD22 binding. Amongst the multiple constructs designed and tested, Loop6 was identified as the most potent format and was further tested in in vivo models.

### LoopCAR6 Efficiently Eradicates CD19posCD22Neg and CD19neg PDX.

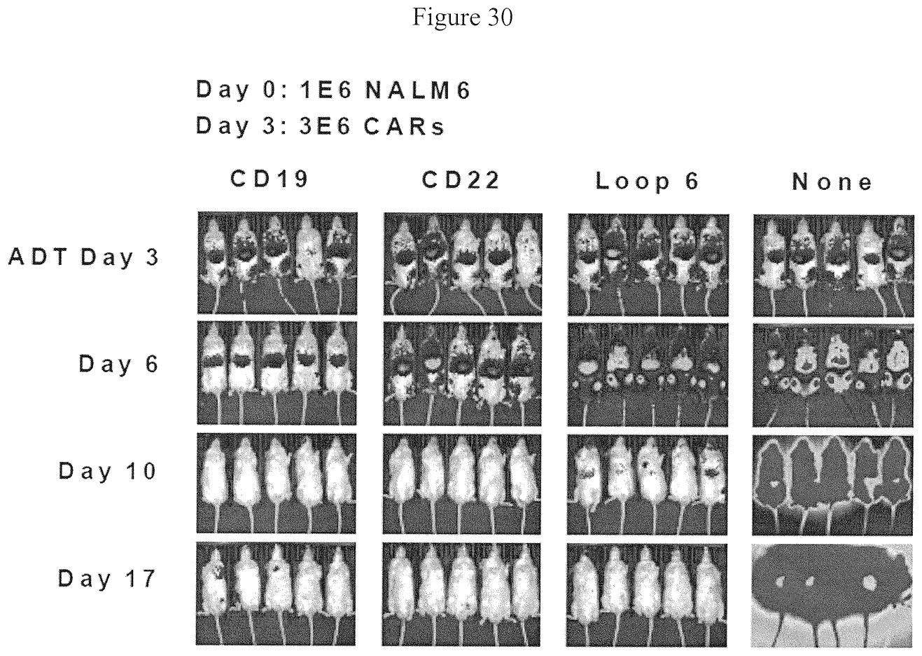

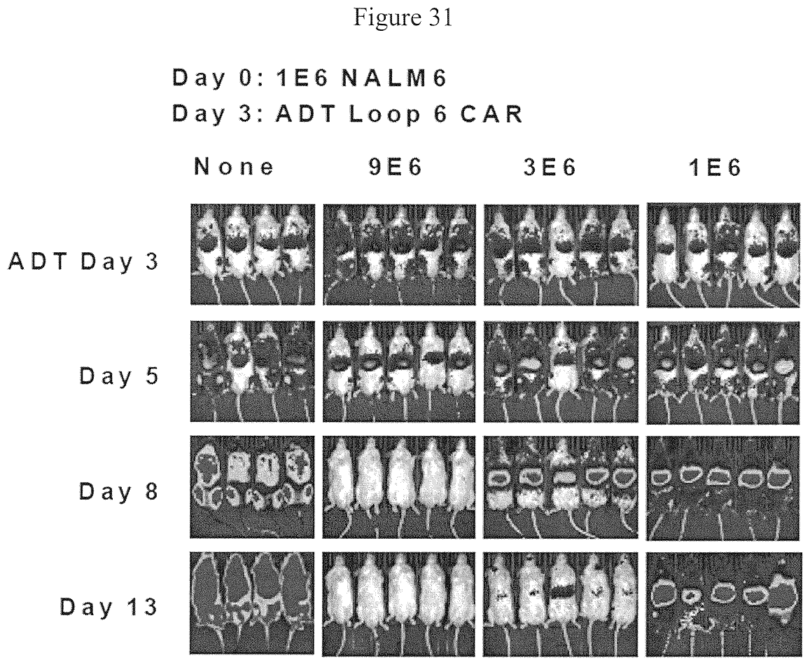

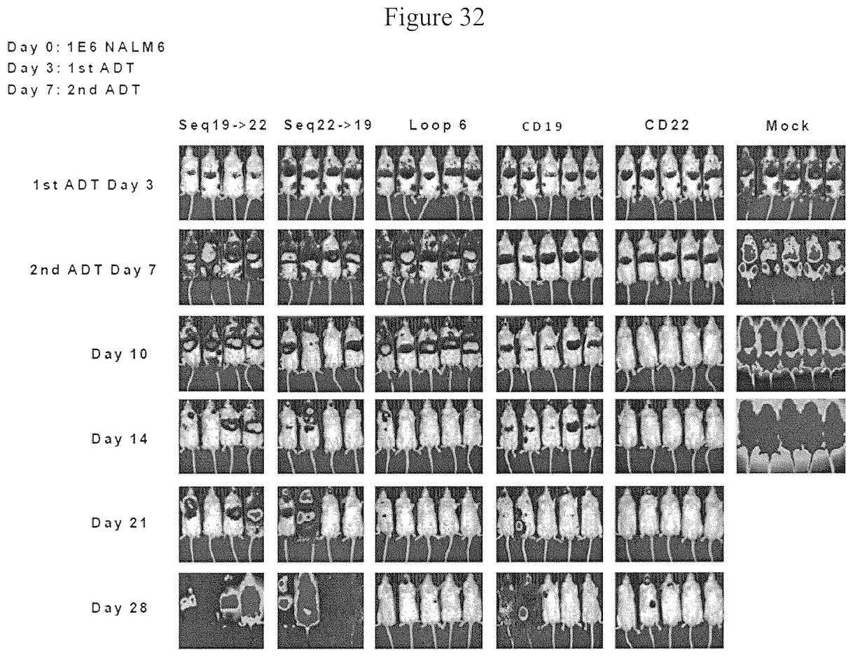

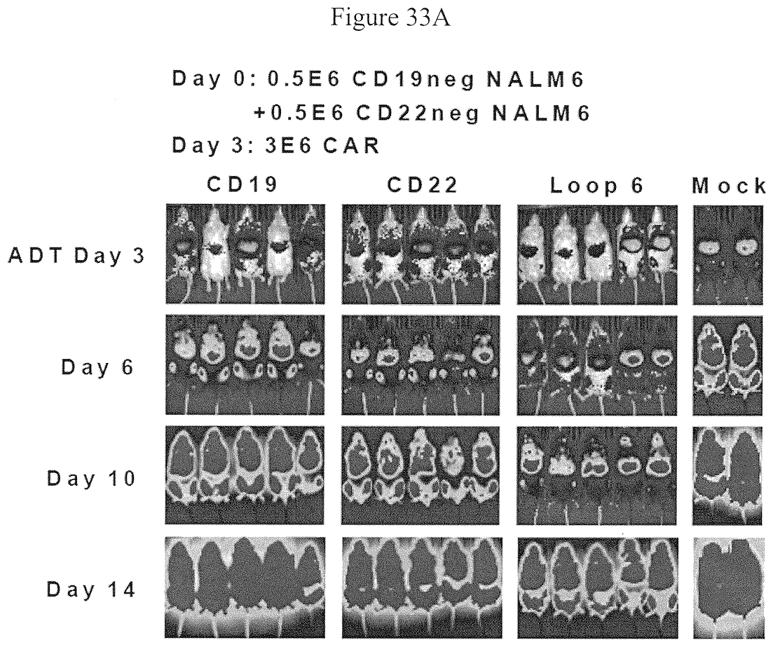

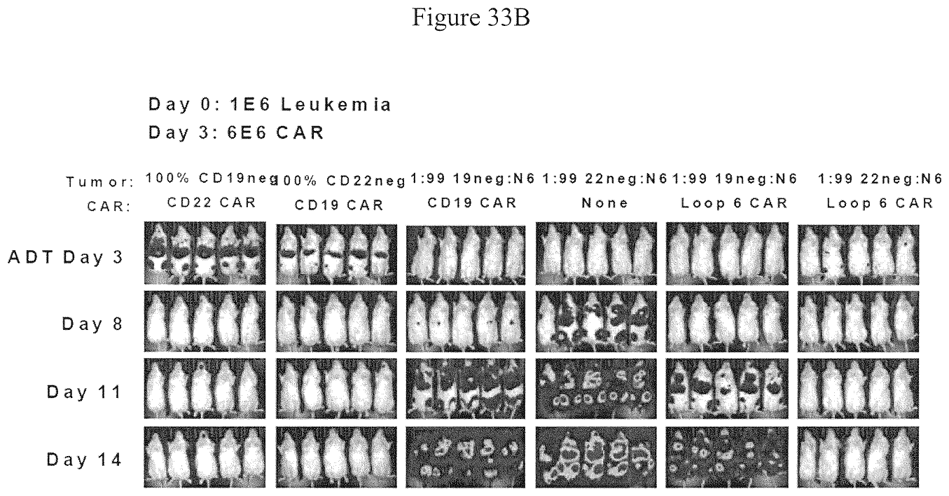

Next tested were LoopCAR6 on Nalm6 xenografts. LoopCAR6 at a dose of 8×10⁶ appear to eradicate CD19pos/CD22pos Nalm6 (FIG. 30) and retained activity down to a dose of 3×10⁶ (FIG. 31). LoopCAR6 was also superior to sequential infusion against CD19pos/CD22pos ALL (FIG. 32). However, at low doses, LoopCAR6 did not work as well against CD19neg/CD22pos leukemia, a cell line with lower expression of CD22 compared to parental NALM6 (FIG. 33A).

LoopCAR6 was further tested in a “spike in” relapse model in which the engrafted ALL inocula contained 1% CD19neg or CD22neg ALL with 99% CD19pos/CD22pos ALL, an assay that mimics relapse from a small, pre-existent clone. In this model, LoopCAR6 was comparable to anti-CD19 CAR at clearing CD22neg ALL, confirming the comparable potency of LoopCAR6 to the anti-CD19 monovalent CARs against CD19. However, in contrast to the anti-CD22 monovalent CAR, LoopCAR6 was unable to completely clear CD19neg/CD22pos ALL with low CD22 site density (FIG. 33B). Collectively, and as suggested by the kinetics of in vitro killing CD22-single expressing ALL (FIG. 27), the in vivo experiments suggest that LoopCAR6 has comparable potency to the anti-CD19 monovalent CAR against CD19 but is slightly less potent than the anti-CD22 monovalent CAR against CD22.

To further explore the in vivo activity of the LoopCAR6 in a clinically relevant model of anti-CD19 CAR resistance, two different patient-derived xenografts generated from de novo relapse specimens (HMB15; CD19pos/CD22pos and HMB28; CD19neg/CD22pos) were utilized. Whole-exome and transcriptome sequencing were performed to characterize the two PDX model systems. HMB15 harbors a translocation that results in an in-frame fusion oncogene between the N-terminus of MLL (exon 1-6) and C terminus of MLLT10. The CD19 and CD22 genomic locus is intact in this model. The HMB28 PDX primary oncogenic driver is a point mutation of KRAS G12D. In addition, this model harbors a premature stop codon in the CD19 (Table 18).

TABLE 18

| PDX Model | Primary Oncogene | CD19 | CD22 |
|---|---|---|---|
| HMB15 | MLL-MLLT10 | Intact DNA and | Intact DNA and |
| | fusion | RNA | RNA |
| HMB28 | KRAS G12D | W214 stop codon | Intact DNA and |
| | | | RNA |

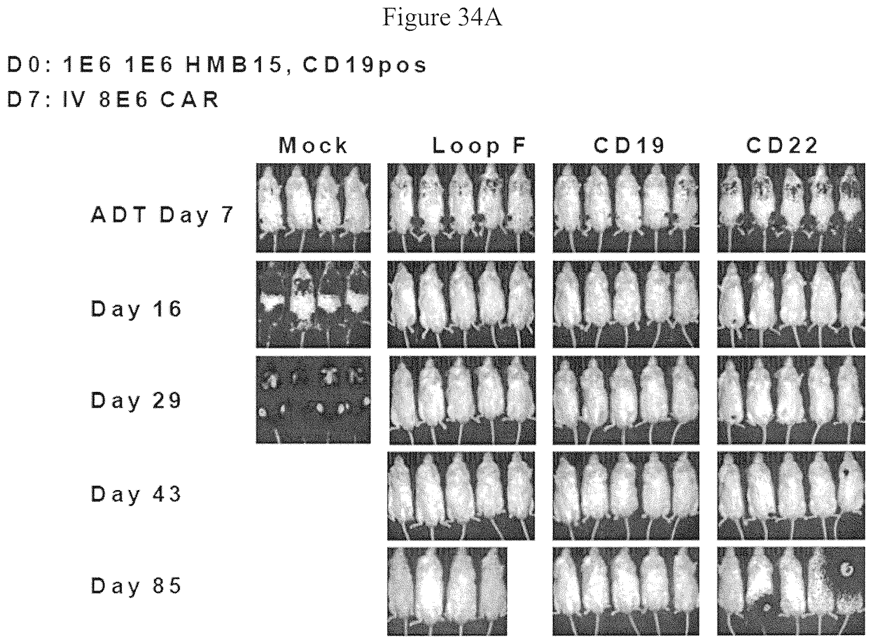

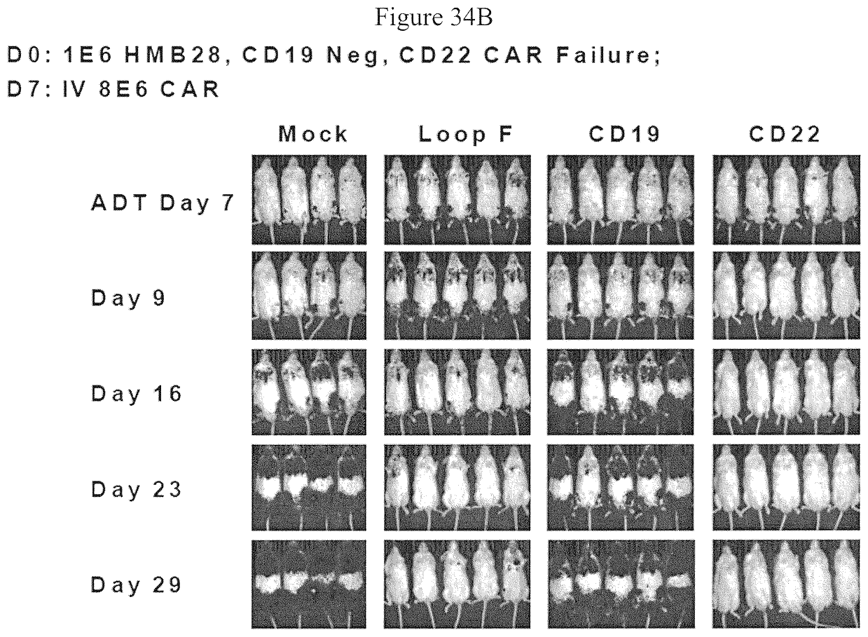

HMB15 appeared to be cleared by both monovalent CARs as well as LoopCAR6 (FIGS. 34A and 34B). HMB28 was resistant to anti-CD19 monovalent CAR and therefore, a good model of anti-CD19 CAR resistance. Encouragingly, LoopCAR6 prevented progression in HMB28, indicating that LoopCAR6 may be effective at preventing anti-CD19 CAR resistance.

### No Evidence for Off Target Activity of LoopCAR6.

Given the possibility of mispairing of two different V_{H} and V_{L} resulting in potential off tumor toxicity, functional screening of T cells expressing the LoopCAR6 was performed. LoopCAR6 T cells were co-incubated with human iPSC cell lines representing multiple normal tissues and IFNγ production was measured in the culture supernatant. IFNγ production was used to measure reactivity as all of the active CAR constructs developed induce IFNγ. NALM6 and REH-TSLPR, two separate ALL cell lines expressing both CD19 and CD22, were used as positive controls.

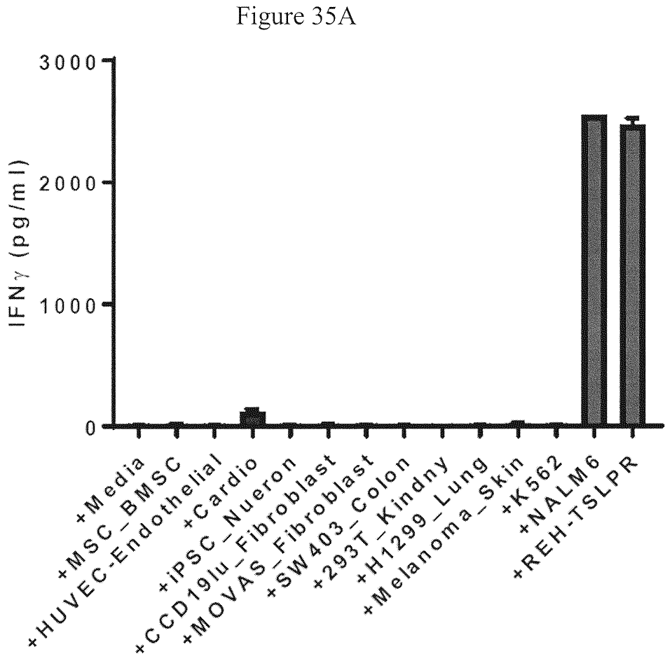

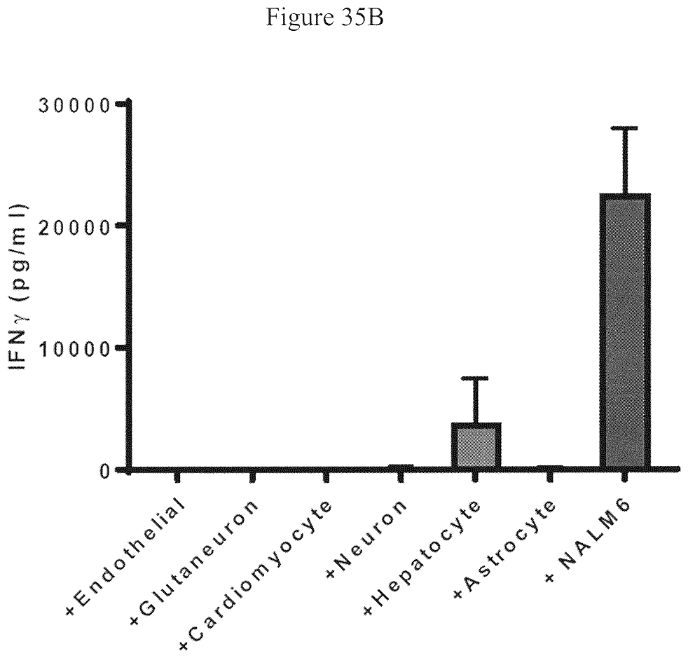

In this assay, LoopCAR6 induced IFNγ in T cells against both NALM6 and REH-TSLPR. IFNγ production was not detected in supernatants of by LoopCAR6-expressing T cells in the presence of any of the iPS cell lines (FIGS. 35A and 35B).

Table 19 presents a summary of results.

TABLE 19

| Construct | Surface Expression | In Vitro Efficacy | In Vivo Efficacy |
|---|---|---|---|
| TanCAR1 | 60% of both anti- | CD19 ++ | ++ |
| | CD19 and anti-CD22 | CD22 | |
| TanCAR2 | 29% anti-CD19 | CD19 + | + |
| | and anti-CD22 | | |
| TanCAR3 | None | | |
| TanCAR4 | 56% of both anti- | CD19 ++ | +++ |
| | CD19 and anti-CD22 | CD22 ++ | |
| LoopCAR1 | 19% of both anti- | CD19 + | |
| | CD19 and anti-CD22 | CD22 | |
| LoopCAR2 | 42% of both anti- | CD19 ++ | |
| | CD19 and anti-CD22 | CD22 | |
| LoopCAR3 | 24% of both anti- | CD19 ++ | |
| | CD19 and anti-CD22 | CD22 ++ | |
| LoopCAR4 | 63% of both anti- | CD19 ++ | +++ |
| | CD19 and anti-CD22 | CD22 +++ | |
| LoopCAR5 | 49% of both anti- | CD19 + | |
| | CD19 and anti-CD22 | CD22 | |
| LoopCAR6 | 82% of both anti- | CD19 +++ | ++++ |
| | CD19 and anti-CD22 | CD22 ++ | |

### Example 8

This example demonstrates cleavable CARs, in accordance with embodiments of the invention.

### Cell Lines and Culture Conditions

The following leukemia cell lines were used: the erythroleukemia K562-CD22 (transduced with human CD22, GeneCopoeia, Cat: EX-Z9364-Lv151), K562-CD19 (transduced with human CD19), K562-CD19CD22 (transduced with both human CD19 and CD22), non-transduced K562 as a negative control; the B cell acute lymphoblastic leukemia lines NALM6, NALM6-GL (transduced with GFP and Luciferase), NALM6-CD19⁻-GL (Crisper KO CD19 on exon 3), NALM6-CD22⁻-GL (Crisper KO CD22 on exon 6). Cell lines were cultured in media supplemented with 10% heat-inactivated FBS, 10 mM HEPES, 100 U/mL penicillin, 100 μg/mL streptomycin, and 2 mM L-glutamine (Invitrogen). The Lenti-X 293T lenti packaging cell line (Clontech. Cat #632180) was cultured in DMEM (Invitrogen) media.

### Primary Human Leukemia Sample and Patient-Derived Xenografts

Human ALL (Acute Lymphoblastic Leukemia) samples were collected and stored after informed consent to an IRB-approved tissue acquisition protocol (Protocol number: 15-C-0029). All research specimens from human subjects were obtained with informed consent in accordance with the Declaration of Helsinki. The following primary samples were used: CD19″ ALL and the CD19⁺CD22^{dim} (de novo relapse specimens ALL_H0113_post22_r (CAR3), ALL_H0090_post19_pd (HMB15), were used for in vivo testing of the bispecific CAR constructs. PDX models were created by injecting 1E6 to 10E6 of the patient ALL cells intravenously into NSG mice (NOD scid gamma, NOD.Cg-Prkdescid Il2rgtm1Wjl/SzJ; Jackson ImmunoResearch Laboratories). The PDX lines were transduced with lenti-GFP-Luc virus and sorted for the leukemia cell expressing GFP and luciferase after the first and second passages. For these studies, the secondary and later passages of the PDX were used for relapse and de novo ALL specimens, respectively. GFP-transduced PDX leukemia in vivo burden was assessed by weekly fluorescence imaging, and animals were treated with CAR T cells via tail vein injection once the human ALL was detectable by florescence imaging. Elutriated human lymphocytes from healthy donors were obtained from the Department of Transfusion Medicine at the National Institutes of Health (NIH) Clinical Center under an IRB-approved protocol. The human lymphocytes were cultured in AIM-V media.

Generating CD19 Negative or CD22 Negative Leukemia with CRISPR

Lentiviral vector for CRISPR knockout of the CD19 or CD22 on NALM6 were made. Briefly, guide-RNAs were optimized by http://crispr.mit.edu/, cloned into LentiCRISPR v2 plasmid (Addgene Plasmid 52,961). Plasmids were then co-transfected with packaging plasmids and transformed into HEK293T cells. Two days later, CRISPR supernatants were harvested, filtered, and concentrated. For viral transduction, 105 NALM6 cells were incubated with 10 ml of concentrated viral supernatant for 2 days, followed by expansion in RPMI media. Cell phenotype was assessed by flow cytometry, followed by sorting of cells with phenotypic alterations and single-cell cloning. Sequencing was performed on single-cell clones to confirm genotypic alterations.

### Making of Lenti-Viral CAR Constructs

CD19/CD22 bicistronic CARs were made with different pairings of the CD28 or 4-1BB costimulation domains in each CD19 and CD22 CAR and linked with a cleavable linker in between. Each CD19 and CD22 CAR has a leader sequence at the beginning, and followed with the CD19 or CD22 single chain variable fragment, then, either a CD8 transmembrane domain linked with 4-1BB and CD3 zeta domain, or a CD28 transmembrane domain linked with CD28 and CD3 zeta domain. These CARs were subcloned into an pELNS lenti vector backbone. All restriction enzymes were purchased from New England Biolabs. The sequence of all CAR constructs was confirmed by sequencing at Macrogen.

The CARs described in this Example are the following: 22-BB/19-28 (which is also listed herein as V5), 22-28/19-BB (which is also listed herein as V6), 22-BB/19-BB (which is also listed herein as V7), and 22-28/19-28 (which is also listed herein as V8).

### CAR T-Cell Generation

The bicistronic CAR-encoding lentiviral vectors were produced by transient transfection of the Lenti-X 293T lenti packaging cell line. Briefly, lenti-X 293T cells were plated into poly-D lysine coated 15-cm plates (BD Biosciences). The following day, Lenti-X 293T cells were transfected using lipofectamine 3000 (Thermo Fisher Scientific) with plasmids encoding the CAR construct along with packaging and envelope vectors (pMDLg/pRRE, pMD-2G, and pRSV-Rev). Lentiviral supernatants were harvested at 24 and 48 hours post-transfection, centrifuged at 3000 RPM for 10 minutes to remove cell debris, frozen on dry ice and stored at −80° C. Human PBMCs from normal donors were obtained with an NIH-approved protocol and activated with a 1:3 ratio of CD3/CD28 microbeads (Dynabeads Human T-Expander CD3/CD28, Thermo Fisher Scientific, Cat #11141D) in AIM-V media containing 40 IU/mL recombinant IL-2 and 5% FBS for 24 hours. Activated T cells were resuspended at 2 million cells per 2 mL of lentiviral supernatant plus 1 mL of fresh AIM-V media with 10 mg/mL protamine sulfate and 100 IU/mL IL-2 in 6-well plates. Plates were centrifuged at 1000×g for 2 hours at 32° C. and incubated overnight at 37° C. A second transduction was performed on the following day by repeating the same transduction procedure described above. The CD3/CD28 beads were removed on the third day following transduction, and the cells were cultured at 300,000 cells/mL in AIM-V containing 100 IU/mL IL2 with fresh IL2-containing media added every 2-3 days until harvest on day 8 or 9.

### Flow Cytometry

Surface expression of CD22 CAR-transduced T cells was determined by flow cytometry using a CD22-Fc (R&D Systems) followed by incubation with PE-F (ab) 2 or APC-F (ab) 2 specific for human IgG-Fc (Jackson ImmunoResearch Laboratories). Surface expression of CD19 CAR-transduced T cells was detected with anti-CD19 Idiotype or Recombinant Human CD19 Fc Chimera Protein (R&D Systems) conjugated with APC by using Lightning-Link APC Antibody Labeling Kit (Novus Biologicals). Expression of CD19, CD22 on B-ALL lines were detected using the following anti-human antibodies: CD45-PerCP-Cy5.5 (eBioscience), CD19-Pacific Blue, CD19-APC-Cy7, CD10-PE-Cy7, and CD22-PE (Biolegend). T cells were characterized with the following antibodies: CD3-APC-Cy7, CD4-Pacific Blue, and CD8a-PE-Cy7 (BioLegend).

### Cytotoxicity Assay

5E4 of Target tumor cells in 100 ul of RPMI media were loaded into a 96-well plate (Corning® BioCoat™ Poly-L-Lysine 96-Well Clear TC-Treated Flat Bottom Assay Plate). An equal amount of CAR T cells were added into the designated well on the following day. The initial incucyte apoptosis marker (Essen BioScience) was diluted in 100 ul PBS and 1 ul of the diluent was added into each well. The plate was scanned for the GFP and or RFP fluorescent expression to monitor the cell apoptosis using an IncuCyte ZOOM® system every 30 minutes in a duration of 40 hours. The percentage of cell killing at each time point was baseline-corrected.

### Analysis of Cytokine Production

Target tumor cell and transduced CAR positive T cells were washed 3 times with 1 XPBS and resuspend in RPMI at 1E6/ml. 100 ul of tumor cells with 100 ul of CAR positive T cells were loaded into each well of a 96-well plate. T cell only and tumor cell only controls were set up. All tests were performed in duplicate or triplicate. Cells were incubated for 18 hours at 37° C. and 120 ul of the culture supernatant was harvested for detection of cytokine production. Cytokine levels in supernatants were measured using either ELISA kits (R&D Systems) or a multiplex assay (Meso Scale Discovery).

### RNAseq Analysis

NALM6 and CAR T cells were resuspended at 1E6/ml. 5E5 of NALM6 was co-incubated with 5E5 CAR+ T cells in 10 ml of AIMV with 40U of IL2 culture media in 25 ml culture flasks for 24 hours in duplicate or triplicate. NALM6 with CD19 microbeads were removed with an LD column. Total effluent was collected and the cells pelleted down by centrifuge at 1200 rpm for 6 minutes. tRNA was extracted immediately with RNAeasy Plus Mini Kit. RNA samples were sent to the NIH core facility for analysis. The RNA quality was evaluated with TapeStation Analysis Software (Agilent Technologies). The RNAseq was generated with NextSeq FASTQ by TruSeq LT assay.

### Bioenergetic Analyses

For the glycolysis stress test, the CAR T cells were suspended in serum-free unbuffered DMEM medium (Sigma-Aldrich) supplemented with L-glutamine (200 mM) and NaCl (143 mM). 0.6 mL of a 0.5% Phenol Red solution (SigmaP0290) was added for a final concentration of 3 mg/L and adjust the pH to 7.35+/−0.05. CAR T Cells were plated onto Seahorse cell plates (3E5 cells per well), coated with Cell-Tak (Corning) to facilitate T cell attachment. Briefly, the cartridges were hydrated the day before the assay. On the day of the assay, the plates were coated with Cell-Tak and the cells were seeded in the Cell-Tak coated plates and placed on the XF24 Analyzer for the assay. The detailed procedure is as the following. The assay cartridge was initially hydrated with XF calibrant solution at 200 ul/well, hydro booster was added, and warped in parafilm, and the sensor cartridge was placed on top of utility plate, and incubated at 37° C. without CO₂ for overnight. The cell culture plate was then coated with Cell-Tak as follows: For 1 plate, 46 μl of Cell-Tak was diluted in 204 μl TC water and 1 ml of NaHCO₃. The mixer was dispensed 50 μl in each well and the plate was incubated at room temperature for at least 20 minutes. After removing the Cell-Tak solution, 250 ml of TC water was used to wash each well. CAR T cells (3E5/well) were plated in 158 μl assay media. The cell culture plate was then spun at 450 rpm for 1 sec at slow acceleration and no deceleration, and then the plate was reversed in orientation and spun at 650 rpm for 1 sec at slow acceleration and no deceleration. The plate was then incubated at 37° C. 0% CO₂ for 25-30 minutes. After 25-30 minutes incubation, added 158 μl of warm assay medium slowly and gently to the top of each well along the side of the wall using a manual P200 pipettor. The cell plates were incubated for 15-25 minutes. After 15-25 minutes, the plates were placed on XF24 Analyzer (after calibration finished). The XF assay were executed. Solution was injected sequentially through three ports: Port A: glucose 80 mM (96 μl of the stock solution in 3 ml assay media). Port B: oligomycin 18 μM (10.8 μl of the stock solution in 3 ml assay media). Port C: 2DG use stock solution. Glycolysis stress test was performed by measuring ECAR (mpH/min) at steady state after the cartridge ports were loaded with 75 μl of drug solution. For the mitochondrial stress test, CAR T cells were suspended in serum-free unbuffered DMEM medium with D-glucose (25 mM), and sodium pyruvate (1 mM). Mitochondrial stress test was performed similarly as the above by measuring OCR (pmol/min) at steady state and after sequential injection of oligomycin (0.5 μM), FCCP (0.5 μM), rotenone (1 μM) and antimycin A (1 μM) (Sigma-Aldrich). Experiments with the Seahorse system utilized the following assay conditions: 2 minutes mixture; 2 minutes wait; and 3 minutes measurement.

### Fluorescence Microscopy Imaging and Analysis

T cells were co-transduced to express CAR-Cerulean or CAR-mCherry fusion proteins. CAR positive T cells were sorted and stained with the lipophilic tracer-DiD Membrane dye (Life Technologies) and with the LIVE/DEAD Fixable Blue Dead Cell (Life Technologies) in PBS. Cells were then washed and mounted. The images were acquired with a Zeiss Apotome fitted with an AxioCam MRm camera, using a Zeiss plan apochromat 20× objective. The exposure setting was the same for the entire experiment. ImageJ software was used for data analysis. Dead cells were excluded. The DiD membrane stain was used to identifying for each cell. The dimension and the maximum intensity of the Cerulean (CFP) positive or mCherry positive region were counted. Only a maximum intensity greater than one was counted. The threshold for the DiD stain was set at 10% of maximum pixel intensity. The threshold for the Cerulean channel and mCherry channel was set at 20% of maximum pixel intensity.

### In Vivo Experiments

Animal experiments were carried out under protocols approved by the NCI Bethesda Animal Care and Use Committee. B-ALL cell lines and the xenografted human B-ALL specimens were IV injected into NSG mice. For luciferase-expressing lines, leukemia was detected using the Xenogen IVIS Lumina (Caliper Life Sciences). NSG were injected intraperitoneally with 3 mg D-luciferin (Caliper Life Sciences) and were imaged 4 minutes later with an exposure time of 30 sec for NALM6 cell and 2 minutes for PDXs. Living Image Version 4.1 software (Caliper Life Sciences) was used to analyze the bioluminescent signal flux for each mouse as photons/s/cm²/sr. Leukemia burden in non-luciferase expressing xenografts was measured by flow cytometry of peripheral blood, bone marrow, and spleen.

### Statistical Analysis

Statistics analysis were performed using Prism 7.0 software. The plots are presented as mean+/−SD. Statistical Significance of all data was calculated using Mann Whitney test for patient CD19 and CD22 analyses.

### Development of a Bicistronic CAR

When tested in the extremely aggressive relapse model with low dose of CAR, bivalent LoopCAR6 does not completely eradicate CD19neg and CD22neg Leukemia. After testing 11 different forms of bivalent CAR, it was found that it is difficult to reserve the CD22 activity in the bivalent form.

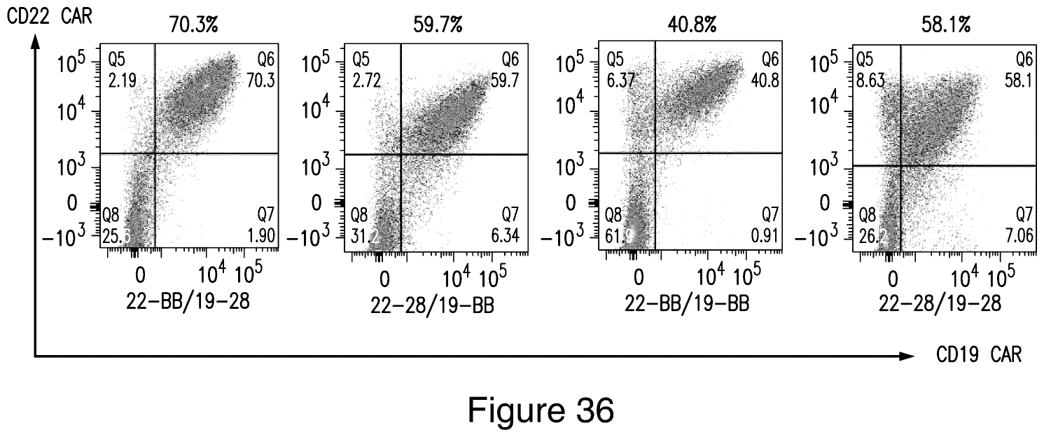

Bicistronic CAR expression was determined by flow cytometry to confirm expression of the bicistronic construct. As indicated in FIG. 36, upon protein translation, bicistronic CD19 CAR and CD22 CAR became two separate fragments and eventually expressed as two CARs on the cell surface. The clustering of cells in the fourth quadrant indicated an equal molar expression of CD19 and CD22 CAR on the cell surface. It was found that the CAR with a 22-BB/19-28 expression system had the highest rate of double positive cells with 70.3% positive cells. The lowest levels of dual expression were associated with those that either double BB or double 28 with 40.8% and 58.1%, respectively. When the costimulatory domains were switched, around 10% expression was lost in the double positive population.

Bicistronic CARs with the Combination of 4-1BB and CD28 have Superior Function In Vitro Compared with Bivalent and Other Bicistronic CARs

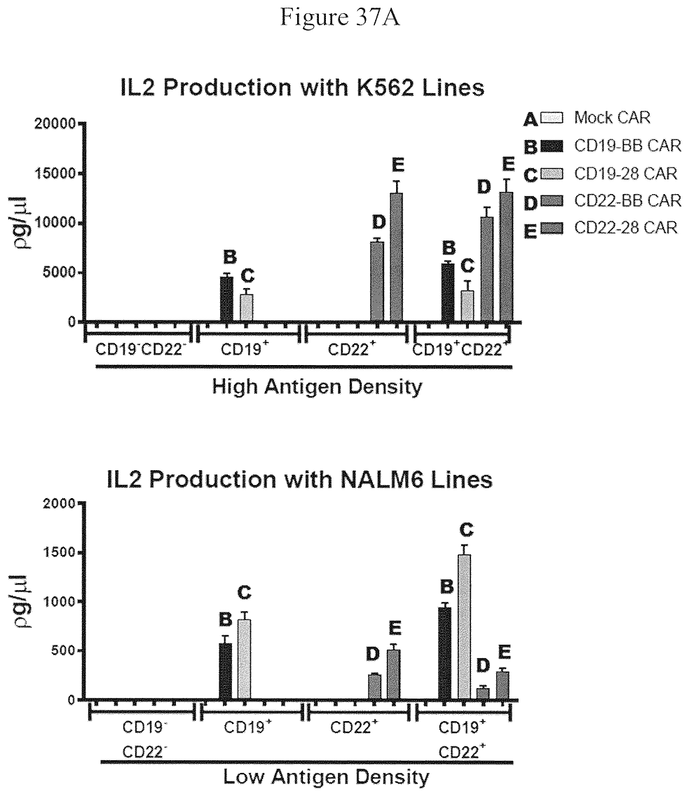

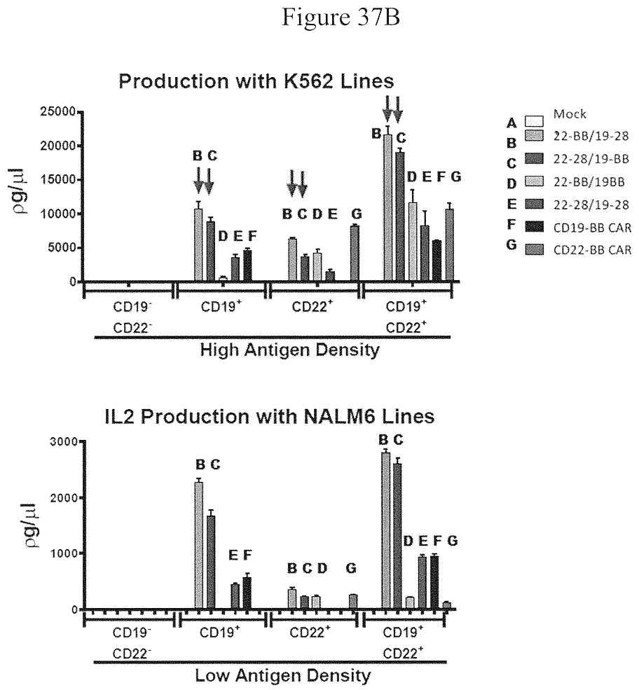

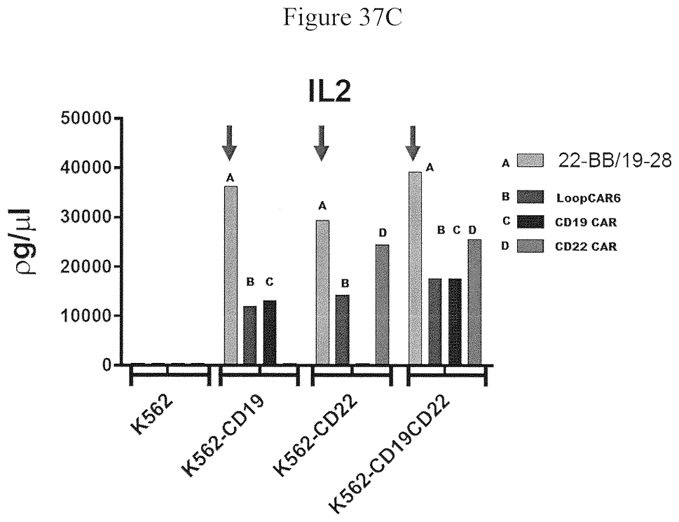

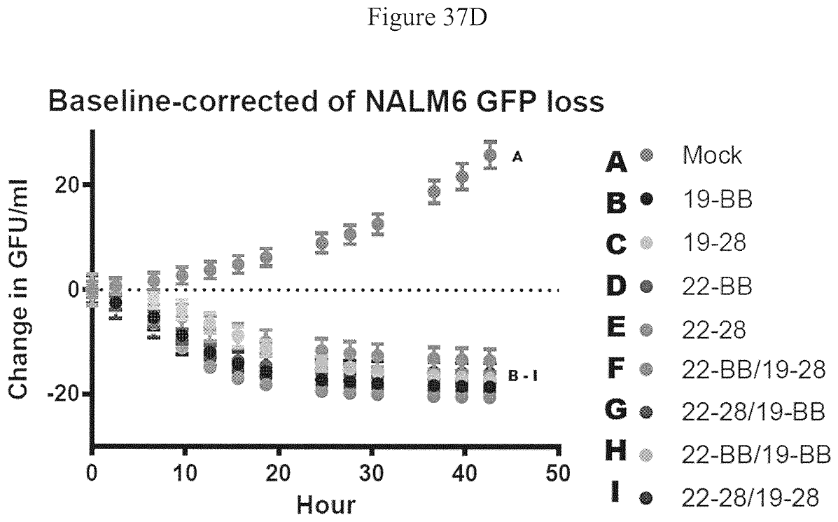

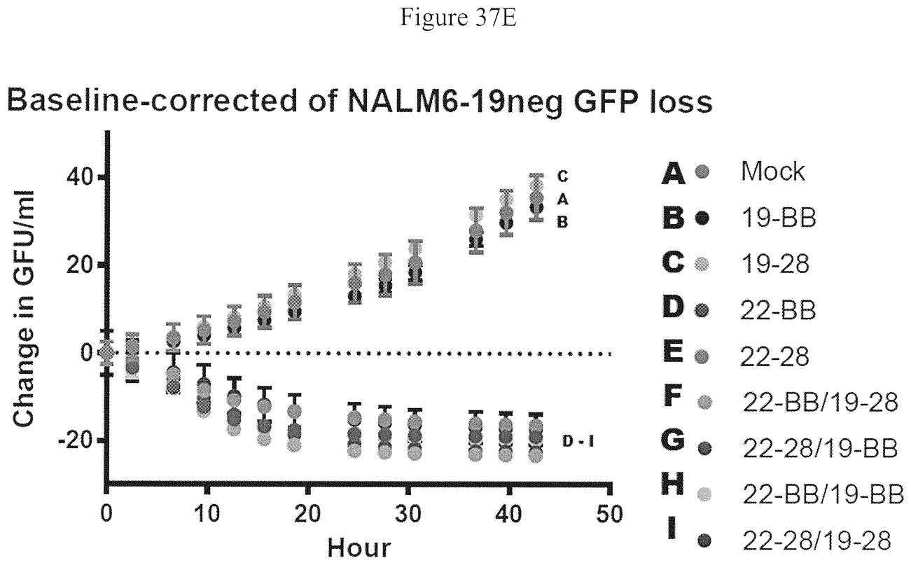

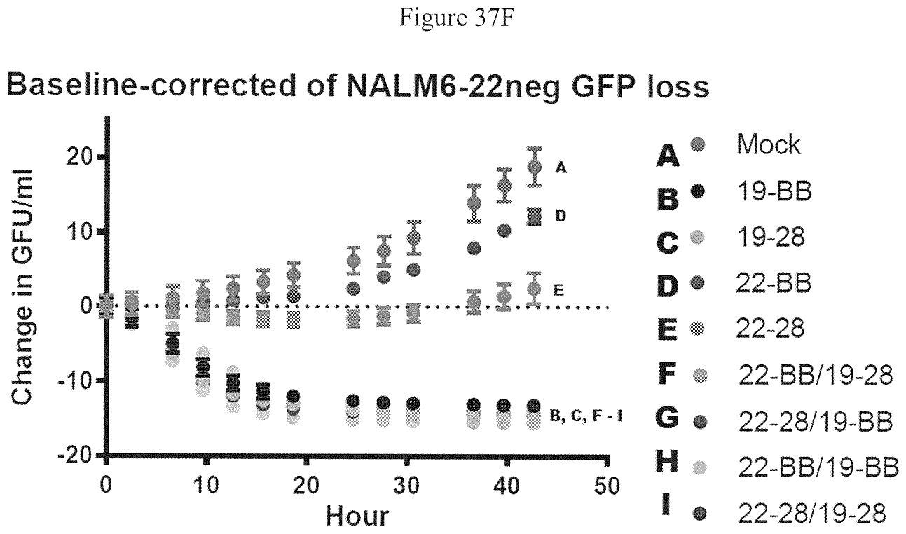

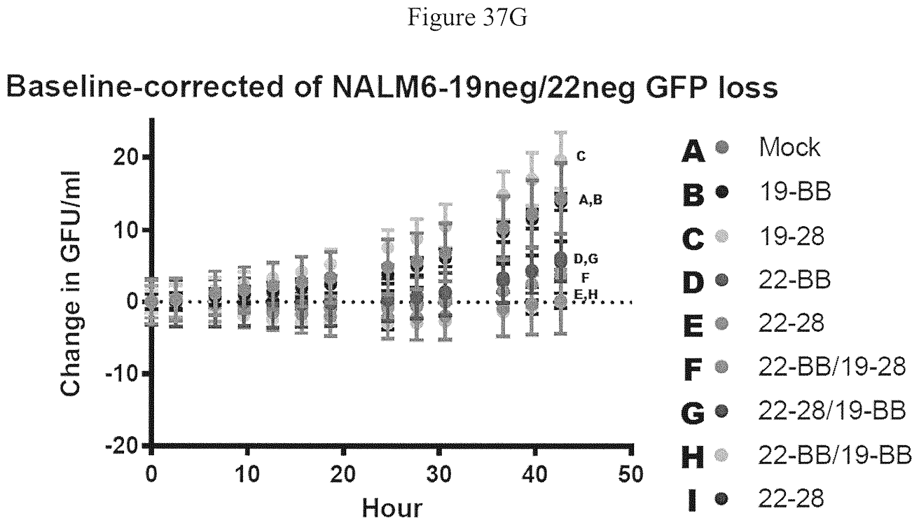

It has been previously reported that co-stimulation endo domain CD28 and 4-1BB has different effects on the immunomodulation of the CAR function. To elucidate the sensitivity of the single targeting CAR constructs, each CAR-T cell was co-cultured with leukemia expressing cognate antigens at various densities, and the IL-2 level was measured in the 18 hr co-culture supernatant. Target antigen density makes as high as 10-fold differences in cytokine production, and CD22 CAR is especially sensitive to the target density (FIG. 37A). The co-stimulation domain also contributes to the difference in cytokine production but the difference attributable to co-stimulation domain was modest compared to the impact of the target antigen density.

Next, bicistronic CARs, having different pairings of the co-stimulation domains, were incubated with K562 and NALM6 derived cells to determine if antigen density would affect cytokine production. The bicistronic CARs with the combination of both CD28 and 4-1BB co-stimulation domain produce more cytokines than the one with only CD28 or 4-1BB co-stimulation domain or the single targeting CAR (FIG. 37B). Antigen density again had the highest impact on the cytokine production of the CAR T cells. 22-BB/19-28 makes slightly more cytokines than the 22-28/19-BB CAR. 22-BB/19-28 CAR also made more cytokines than the CD19/CD22 bivalent CAR (FIG. 37C). All the bicistronic CARs demonstrated effective killing of the target cell lines (FIG. 37D-37G).

RNAseq Analysis Demonstrates Unique Gene Expression Associated with Different Pairing of Costimulatory Domain

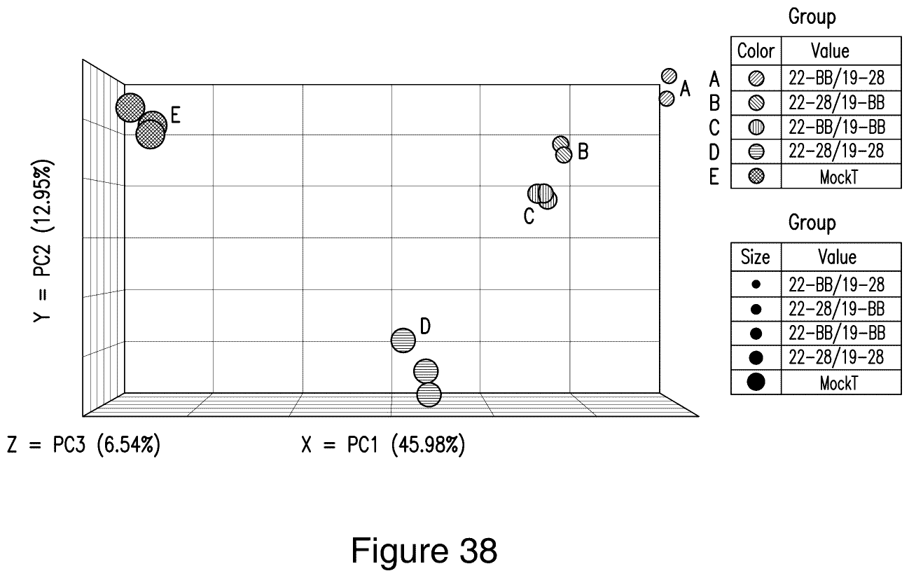

RNAseq analysis was performed to interrogate the biological pathways associated with the different co-stimulation domain combinations. The bicistronic CARs were co-incubated with CD19⁺CD22⁺ NALM6 cells, and extracted total RNA for the RNAseq analysis. The PCA indicates that the different combinations are associated with distinct gene expression profile (FIG. 38).

Bicistronic CAR Efficiently Reduces Both CD19⁺CD22⁺ Leukemia, and CD19^{neg} or CD22^{neg} Leukemia Blast

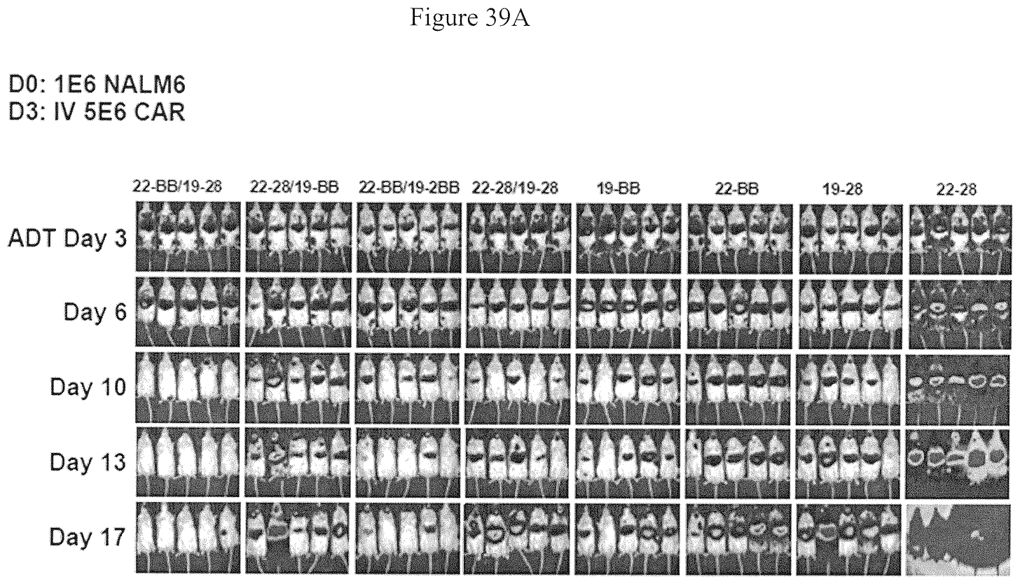

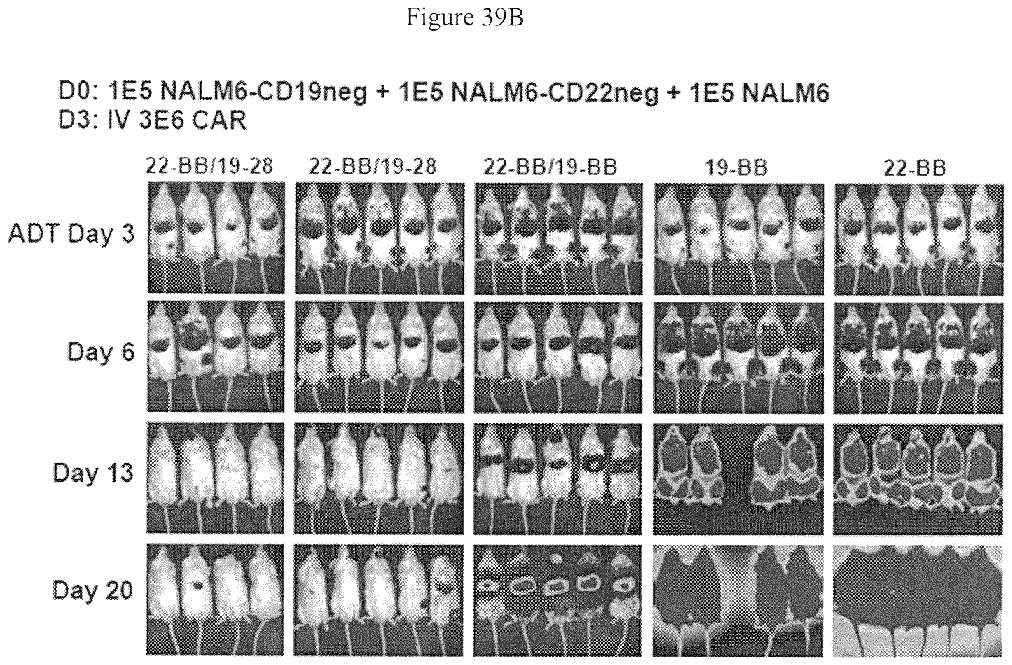

CD19⁺CD22⁺ NALM6 line was used to test the in vivo activity of the bicistronic CARs. CD19 and CD22 CAR with CD28 or 4-1BB single CARs were used as the controls. As indicated in FIG. 39, in general, bicistronic CARs are better than the single targeting CARs. Different pairings of CD28 and 4-1BB costimulation induce different rate of tumor elimination; 22-BB/19-28 is the best one at eliminating the leukemia blast.

CD19^{neg} and CD22low blast have been observed in relapsed patients. To simulate this clinic situation, the CRISPR Cas9 technology was used to generate CD19^{neg} and CD22^{neg} leukemia lines with NALM6 cell. A mixture of the CD19^{neg}, CD22^{neg}, and parental NALM6 cells were injected into immunodeficient NSG to create an aggressive xenograft model to simulate the clinical relapse situation. Three bicistronic CARs were compared to the CD19 and CD22 single targeting CARs (FIG. 39B). Single targeting CARs were not able to prevent the leukemia progression. Bicistronic CARs with both CD28 and 4-1BB had very potent activity on clearing the leukemia.

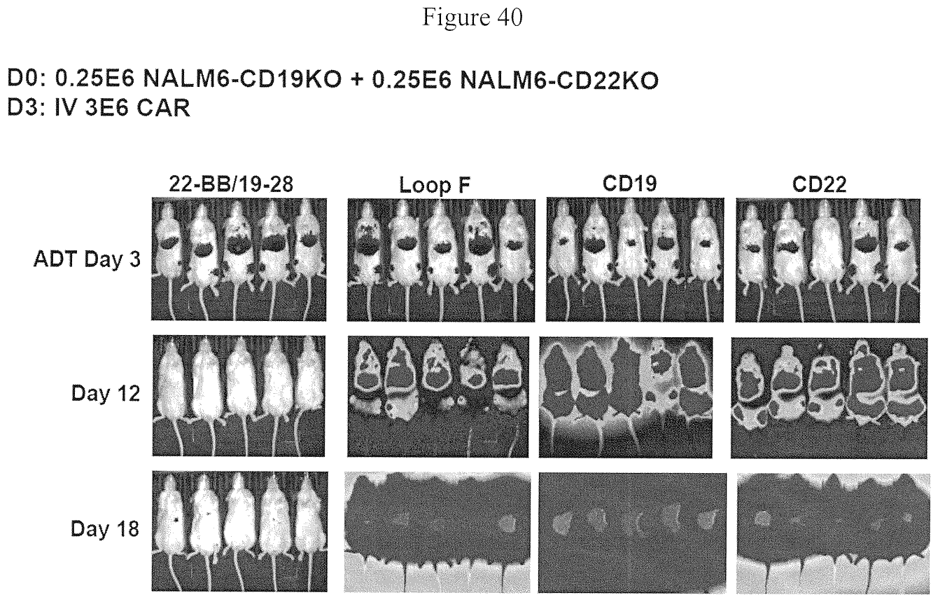

Further comparison was made with the bicistronic CAR and the bivalent CAR in vivo with the mixed CD19^{neg} and CD22^{neg} leukemia (FIG. 40). The bicistronic CAR is superior to the bivalent CARs in reducing the CD19^{neg} and CD22^{neg} leukemia.

### Potent Activities of Bicistronic CAR

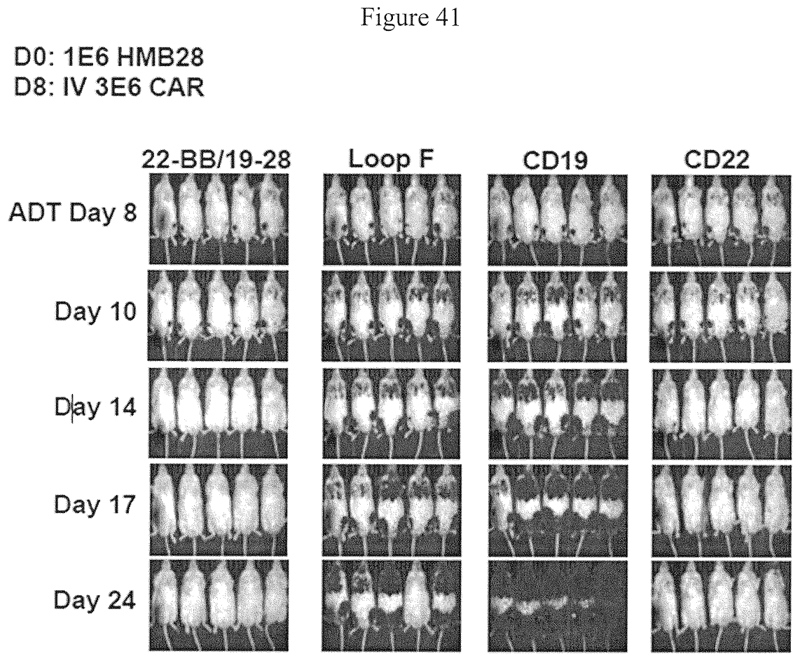

A bicistronic CAR was tested using the clinically relevant CD19^{neg} PDX model (HMB28). Leukemia for the PDX model was derived from a patient who was previously treated with CD19 CAR and relapsed with CD19 negative leukemia relapse, subsequently treated with anti-CD22 CAR-T cells, which failed to clear leukemia due to an emergence of CD22-low expressing blast. Bicistronic CAR can completely eradicate the CD19 negative leukemia blast (FIG. 41).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A chimeric antigen receptor (CAR) amino acid construct comprising:
(a) a cleavable domain;
(b) a first CAR comprising
a first antigen binding domain,
a first transmembrane domain, and
a first intracellular T cell signaling domain; and
(c) a second CAR comprising
a second antigen binding domain,
a second transmembrane domain, and
a second intracellular T cell signaling domain;
wherein the first and second CARs are linked through the cleavable domain,
wherein the first antigen binding domain comprises an antigen binding domain of the m971 antibody,
wherein when the first CAR is cleaved from the construct, the first antigen binding domain has antigenic specificity for CD22.

2-8. (canceled)

9. A chimeric antigen receptor (CAR) amino acid construct comprising:
(a) a cleavable domain;
(b) a first CAR comprising
a first antigen binding domain,
a first transmembrane domain, and
a first intracellular T cell signaling domain; and
(c) a second CAR comprising
a second antigen binding domain,
a second transmembrane domain, and
a second intracellular T cell signaling domain;
wherein the first and second CARs are linked through the cleavable domain,
wherein the first antigen binding domain comprises an antigen binding domain of the FMC63 antibody,
wherein when the first CAR is cleaved from the construct, the first antigen binding domain has antigenic specificity for CD19.

10-22. (canceled)

23. A chimeric antigen receptor (CAR) amino acid construct comprising an amino acid sequence having 90% or greater sequence identity with any one of SEQ ID NOS: 63-70.

24. A chimeric antigen receptor (CAR) amino acid construct comprising:
(a) two or more cleavable domains;
(b) a first CAR comprising
a first antigen binding domain,
a first transmembrane domain, and
a first intracellular T cell signaling domain; and
(c) a second CAR comprising
a second antigen binding domain,
a second transmembrane domain, and
a second intracellular T cell signaling domain;
wherein the first and second CARs are linked through the two or more cleavable domains.

25. The CAR construct of claim 24, wherein the two or more cleavable domains are immediately adjacent or have at least one linker between at least two cleavable domains.

26. The CAR construct of claim 25, wherein there are exactly two cleavable domains.

27-35. (canceled)

36. A method of making a chimeric antigen receptor (CAR) amino acid construct, the method comprising designing two or more cleavable domains between
(a) a first CAR comprising
a first antigen binding domain,
a first transmembrane domain, and
a first intracellular T cell signaling domain; and
(b) a second CAR comprising
a second antigen binding domain,
a second transmembrane domain, and
a second intracellular T cell signaling domain;
wherein the first and second CARs are linked through the two or more cleavable domains; and
cloning into a plasmid a sequence comprising from N-terminus to C-terminus the first CAR, the two or more cleavable domains, and the second CAR.

37. The method of claim 36, wherein the two or more cleavable domains are immediately adjacent or have at least one linker between at least two cleavable domains.

38. The method of claim 36, wherein there are exactly two cleavable domains.
